(19) United States
(12) Patent Application Publication
(10) Pub. No.: US 2026/0192117 A1
(43) Pub. Date: Jul. 9, 2026

(54) METHOD AND APPARATUS FOR ESTABLISHING ATRIAL SYNCHRONOUS VENTRICULAR PACING CONTROL PARAMETERS

(71) Applicant: Medtronic, Inc., Minneapolis, MN (US)

(72) Inventors: Todd J. SHELDON, North Oaks, MN (US); Andrew RADTKE, Minneapolis, MN (US); Keelia Marie ESCALANTE, Minneapolis, MN (US); Aaron M. SAIKIN, Highlands Ranch, CO (US)

(21) Appl. No.: 18/868,469

(22) PCT Filed: May 18, 2023
(86) PCT No.: PCT/IB2023/055127

§ 371 Date: Nov. 22, 2024

Related U.S. Application Data
(60) Provisional application No. 63/346,849, filed on May 28, 2022.

(51) Int. Cl. A61N 1/365; A61N 1/02; A61N 1/05

(52) U.S. Cl. CPC A61N 1/36585; A61N 1/025; A61N 1/056; A61N 1/36507; A61N 1/36578

(57) ABSTRACT

A medical device includes processing circuitry configured to receive a cardiac motion signal and at least one cardiac electrical signal sensed over a signal episode. The processing circuitry is configured to determine that a P-wave of the at least one cardiac electrical signal occurs in a diastolic period of a cardiac cycle of the signal episode. In response to the P-wave being in the diastolic period of the cardiac cycle, the medical device may determine a feature of the cardiac motion signal sensed during the cardiac cycle and, based on the determined feature, establish a control parameter used for controlling delivery of atrial synchronous ventricular pacing.

This application claims the benefit of U.S. Provisional Patent Application Ser. No. 63/346,849 filed 28 May 2022, the entire content of which is incorporated herein by reference.

### TECHNICAL FIELD

This disclosure relates to a method and apparatus for establishing parameters for controlling atrial synchronous pacing functions of a pacemaker.

### BACKGROUND

Implantable cardiac pacemakers are often placed in a subcutaneous pocket and coupled to one or more transvenous medical electrical leads carrying pacing and sensing electrodes positioned in the heart. A cardiac pacemaker implanted subcutaneously may be a single chamber pacemaker coupled to one transvenous medical lead for positioning electrodes in one heart chamber, atrial or ventricular, or a dual chamber pacemaker coupled to two intracardiac leads for positioning electrodes in both an atrial and a ventricular chamber. Multi-chamber pacemakers are also available that may be coupled to three leads, for example, for positioning electrodes for pacing and sensing in one atrial chamber and both the right and left ventricles.

Intracardiac pacemakers have been proposed that are implantable within a ventricular chamber of a patient's heart for delivering ventricular pacing pulses. Such a pacemaker may sense R-wave signals attendant to intrinsic ventricular depolarizations and deliver ventricular pacing pulses in the absence of sensed R-waves. While single chamber ventricular sensing and pacing by an intracardiac ventricular pacemaker may adequately address some patient conditions, some patients may benefit from atrial event signal and ventricular event signal sensing (which may be referred to as “dual chamber sensing”) to enable delivery of ventricular pacing pulses to a patient's heart that are synchronized to atrial event signals for promoting a more normal heart rhythm and coordinated atrial and ventricular heart chamber activity.

### SUMMARY

The techniques of this disclosure generally relate to selection of control parameters by processing circuitry of a medical device system for use by a pacemaker configured to deliver atrial synchronous ventricular pacing. The processing circuitry is configured to receive P-wave timing markers and a cardiac mechanical signal and establish control parameters based on analysis of the P-wave timing markers and cardiac mechanical signal. The established control parameters may be put into effect by a ventricular pacemaker configured to sense the cardiac mechanical signal, sense atrial events attendant to atrial contraction from the cardiac mechanical signal and deliver atrioventricular synchronous (AVS) pacing pulses to a patient's heart based on the timing of the sensed atrial events. The pacemaker may have a sensor producing a cardiac mechanical signal, e.g., a motion signal, including ventricular and atrial event signals corresponding to the mechanical activity of the heart, e.g., contraction of the heart chambers.

A medical device system operating according to the techniques disclosed herein determines one or more AVS pacing control parameters used for sensing the atrial event signals and/or controlling pacing pulse delivery by determining a feature of the cardiac mechanical signal during at least one cardiac cycle and establishing the AVS pacing control parameter based on at least the feature of the cardiac mechanical signal. Processing circuitry of the medical device may determine the timing of a P-wave attendant to the electrical depolarization of the atria and reject or accept a cardiac cycle based on the P-wave timing. When the cardiac cycle is accepted, a feature of the cardiac mechanical signal sensed during the accepted cardiac cycle may be used by the processing circuitry for establishing or adjusting an AVS pacing control parameter. When the cardiac cycle is rejected, a feature of the cardiac mechanical signal sensed during the rejected cardiac cycle is not used for establishing or adjusting the AVS pacing control parameter. In some examples, a feature of the cardiac mechanical signal is determined from the cardiac mechanical signal sensed during each of multiple accepted cardiac cycles, and an AVS pacing control parameter is established or adjusted based on the features determined from the accepted cardiac cycles.

In one example, the disclosure provides a medical device including processing circuitry configured to receive a cardiac motion signal sensed over a signal episode and receive at least one cardiac electrical signal. The processing circuitry is configured to determine that a P-wave of the at least one cardiac electrical signal occurs in a diastolic period of a cardiac cycle of the signal episode. In response to the P-wave being in the diastolic period of the cardiac cycle, the processor may determine at least one feature of the cardiac motion signal sensed during the cardiac cycle and establish a control parameter based the determined feature, where the control parameter is used for controlling delivery of atrial synchronous ventricular pacing.

In another example, the disclosure provides a method performed by a medical device. The method includes receiving a cardiac motion signal sensed over a signal episode and receiving at least one cardiac electrical signal. The method can further include determining that a P-wave of the at least one cardiac electrical signal occurs in a diastolic period of a cardiac cycle of the signal episode. In response to the P-wave being in the diastolic period of the cardiac cycle, the method can further include determining at least one feature of the cardiac motion signal sensed during the cardiac cycle and establishing an atrial synchronous ventricular pacing control parameter based on the determined feature.

In another example, the disclosure provides a non-transitory computer readable medium storing instructions which, when executed by processing circuitry of a medical device, cause the medical device to receive a cardiac motion signal sensed over a signal episode and receive at least one cardiac electrical signal. The instructions further cause the medical device to determine that a P-wave of the at least one cardiac electrical signal occurs in a diastolic period of a cardiac cycle of the signal episode. In response to the P-wave being in the diastolic period of the cardiac cycle, the instructions further cause the medical device to determine at least one feature of the cardiac motion signal sensed during the cardiac cycle and establish a control parameter based on the determined feature, where the control parameter is used for controlling delivery of atrial synchronous ventricular pacing.

In another example, the disclosure provides a medical device including a motion sensor configured to sense a cardiac motion signal, a pulse generator configured to generate ventricular pacing pulses and a telemetry circuit configured to transmit a signal episode of the motion signal. The telemetry circuit may be configured to receive an established control parameter from a second device. The medical device may further include a control circuit configured to operate in an atrial synchronous ventricular pacing mode according to the established control parameter by sensing atrial events from the cardiac motion signal and controlling the pulse generator to deliver atrial synchronous ventricular pacing pulses in response to sensing the atrial events. The control circuit may be further configured to determine that a percentage of atrial synchronous ventricular pacing pulses out of a plurality of ventricular events is greater than a threshold percentage and confirm the established control parameter for use in controlling atrial synchronous pacing.

Further disclosed herein is the subject matter of the following clauses:

Clause 1. A medical device comprising processing circuitry configured to:
receive a cardiac motion signal sensed over a first signal episode;
receive at least one cardiac electrical signal;
determine that a first P-wave of the at least one cardiac electrical signal occurs in a diastolic period of a first cardiac cycle of the first signal episode;
in response to the first P-wave being in the diastolic period of the first cardiac cycle, determine at least a first feature of the cardiac motion signal sensed during the first cardiac cycle; and
establish a first control parameter based on at least the first feature, the first control parameter used for controlling delivery of atrial synchronous ventricular pacing.

Clause 2. The medical device of clause 1, wherein the processing circuitry comprises a cardiac signal analyzer, the processing circuitry being further configured to input the at least one cardiac electrical signal to the cardiac signal analyzer,
output a P-wave timing marker by the cardiac signal analyzer in response an identified P-wave; and
determine that the first P-wave is in the diastolic period of the first cardiac cycle based on the P-wave timing marker output by the cardiac signal analyzer.

Clause 3. The medical device of any of clauses 1-2, wherein the processing circuitry is further configured to:
receive the cardiac motion signal and the at least one cardiac electrical signal sensed over a plurality of cardiac cycles of the first signal episode;
identify a plurality of P-waves including the first P-wave over the plurality of cardiac cycles;
identify at least one diastolic P-wave cycle among the plurality of cardiac cycles, each identified diastolic P-wave cycle being a cardiac cycle that is associated with a P-wave of the identified plurality of P-waves being during a diastolic period of the respective cardiac cycle;
determine a plurality of features of the motion signal comprising the first feature from the plurality of identified diastolic P-wave cycles; and
establish the first control parameter based on the plurality of features.

Clause 4. The medical device of any of clauses 1-3, wherein the processing circuitry is further configured to receive the motion signal over the plurality of cardiac cycles comprising asynchronous ventricular pacing pulses.

Clause 5. The medical device of any of clauses 1-4, wherein the processing circuitry is further configured to:
receive the cardiac motion signal and the at least one cardiac electrical signal sensed over a second signal episode during which atrial synchronous ventricular pacing is delivered according to the established first control parameter;
identify a second P-wave in the at least one cardiac electrical signal;
determine that the second P-wave occurs in a diastolic period of a second cardiac cycle;
in response to the second P-wave being in the diastolic period of the second cardiac cycle, determine at least a second feature of the cardiac motion signal sensed during the second cardiac cycle; and
establish a second control parameter based on at least the second feature, the second control parameter used for controlling the delivery of atrial synchronous ventricular pacing.

Clause 6. The medical device of any of clauses 1-5, wherein the processing circuitry is further configured to establish the first control parameter by establishing an atrial event sensing control parameter used for sensing atrial events from the motion signal.

Clause 7. The medical device of clause 6, wherein the processing circuit is further configured to:
determine the first feature of the cardiac motion signal sensed during the first cardiac cycle by determining a maximum amplitude of the motion signal during the diastolic period of the first cardiac cycle; and
establish the first control parameter based on at least the first feature by establishing a motion signal sensing vector for sensing the motion signal.

Clause 8. The medical device of clause 6, wherein the processing circuit is further configured to:
determine the first feature of the cardiac motion signal sensed during the first cardiac cycle by determining a maximum amplitude of the motion signal during the diastolic period of the first cardiac cycle; and
establish the first control parameter based on at least the first feature by establishing an atrial event sensing threshold amplitude for sensing atrial events from the motion signal.

Clause 9. The medical device of clause 8, wherein the processing circuitry is further configured to:
determine that the first P-wave occurs in a late diastolic period of the first cardiac cycle; and
establish the first control parameter based on at least the first feature by establishing a late atrial event sensing threshold amplitude that is applied to the motion signal during an atrial event window for sensing atrial events from the motion signal.

Clause 10. The medical device of any of clauses 8-9, wherein the processing circuitry is further configured to:
identify a second P-wave in the at least one cardiac electrical signal;
determine that the second P-wave occurs in an early diastolic period of a second cardiac cycle of the first signal episode;
in response to the second P-wave being in the early diastolic period of the second cardiac cycle, determine at least a second feature of the cardiac motion signal sensed during the second cardiac cycle; and
establish a second control parameter based on at least the second feature by establishing an early atrial event sensing threshold amplitude that is applied to the motion signal during a passive ventricular filling window for sensing atrial events from the motion signal.

Clause 11. The medical device of any of clauses 8-9, wherein the processing circuitry is further configured to:
determine that the first P-wave occurs in a late diastolic period of the first cardiac cycle;
determine a second feature of the cardiac motion signal sensed during the first cardiac cycle by determining a maximum amplitude of the motion signal during a passive ventricular filling window of the first cardiac cycle; and
establish a second control parameter based on at least the second feature by establishing an early atrial event sensing threshold amplitude that is applied to the motion signal during the passive ventricular filling window for sensing atrial events from the motion signal.

Clause 12. The medical device of clause 6, wherein the processing circuitry is further configured to:
set a test threshold amplitude;
determine the first feature of the cardiac motion signal sensed during the first cardiac cycle by determining a latest threshold crossing of the test threshold amplitude during a passive ventricular filling window of the first cardiac cycle; and
establish the first control parameter based on at least the first feature by establishing a passive ventricular filling window ending time used for sensing atrial events from the motion signal.

Clause 13. The medical device of clause 6, wherein the processing circuitry is further configured to:
determine the first feature of the cardiac motion signal sensed during the first cardiac cycle by determining a time of a maximum amplitude of the cardiac motion signal during the diastolic period of the first cardiac cycle; and
establish the first control parameter based on at least the first feature by establishing a passive ventricular filling window ending time used for sensing atrial events from the motion signal.

Clause 14. The medical device of clause 6, wherein the processing circuitry is further configured to:
determine the first feature of the cardiac motion signal sensed during the first cardiac cycle by determining a maximum amplitude of the motion signal during the first cardiac cycle; and
establish the first control parameter based on at least the first feature by establishing a post-ventricular atrial blanking period.

Clause 15. The medical device of any of clauses 1-14, wherein the processing circuitry is further configured to:
receive the cardiac motion signal and the at least one cardiac electrical signal sensed over a plurality of cardiac cycles of the first signal episode;
identify a plurality of P-waves over the plurality of cardiac cycles;
determine at least one PP interval between consecutively identified P-waves of the plurality of P-waves; and
establish a second control parameter based on the at least one PP interval for controlling delivery of atrial synchronous ventricular pacing.

Clause 16. The medical device of clause 15, wherein the processing circuitry is further configured to establish the second control parameter by establishing at least one of: a pacing lower rate, a rate smoothing increment, a post-ventricular atrial blanking period, and a maximum atrial tracking rate.

Clause 17. The medical device of any of clauses 1-16, wherein the processing circuitry is further configured to:
determine that a threshold percentage of atrioventricular synchronous pacing pulses is not met; and
in response to determining that the threshold percentage of atrioventricular synchronous pacing pulses is not met, adjust at least one atrial synchronous pacing control parameter.

Clause 18. The medical device of clause 17, wherein the processing circuitry is further configured to adjust the at least one atrial synchronous pacing control parameter by adjusting one of a plurality of control parameters that includes the first control parameter.

Clause 19. The medical device of any of clauses 17-18, wherein the processing circuitry is further configured to adjust the at least one control parameter by adjusting at least one of:
a post-ventricular atrial blanking period, a passive ventricular filling window ending time, an early atrial event sensing threshold amplitude, or a late atrial event sensing threshold amplitude.

Clause 20. The medical device of any of clauses 17-19, wherein the processing circuitry is further configured to adjust the at least one control parameter by adjusting at least one of: a pacing lower rate, a rate smoothing increment, a maximum atrial tracking rate.

Clause 21. The medical device of any of clauses 1-20, wherein the processing circuitry is further configured to determine that the first P-wave occurs in a diastolic period of the first cardiac cycle of the first signal episode by:
applying a late diastolic threshold time; and
determining that the first P-wave occurs after the late diastolic threshold time and before a ventricular electrical event that ends the first cardiac cycle.

Clause 22. The medical device of any of clauses 1-21, wherein the processing circuitry is further configured to:
receive the cardiac motion signal and the at least one cardiac electrical signal sensed over a plurality of cardiac cycles of the first signal episode;
determine from the at least one cardiac electrical signal that less than a threshold number of the plurality of cardiac cycles are identified as diastolic P-wave cycles; and
adjust at least one asynchronous ventricular pacing interval in response to less than the threshold number of the plurality of cardiac cycles being identified as diastolic P-wave cycles.

Clause 23. The medical device of any of clauses 1-22, wherein the processing circuitry is further configured to:
determine a low confidence of P-wave identification in the at least one cardiac electrical signal; and
establish the first control parameter based on the cardiac motion signal sensed over a plurality of cardiac cycles.

Clause 24. The medical device of any of clauses 1-23, further comprising a telemetry circuit configured to transmit a programming command comprising the established first control parameter.

Clause 25. The medical device of any of clauses 1-23, further comprising a pulse generator configured to deliver atrial synchronous ventricular pacing according to the first control parameter.

Clause 26. A non-transitory computer readable medium storing instructions which, when executed by processing circuitry of a medical device, cause the medical device to:
receive a cardiac motion signal sensed over a first signal episode;
receive at least one cardiac electrical signal;
determine that a first P-wave of the at least one cardiac electrical signal occurs in a diastolic period of a first cardiac cycle of the first signal episode;
in response to the first P-wave being in the diastolic period of the first cardiac cycle, determine at least a first feature of the cardiac motion signal sensed during the first cardiac cycle; and
establish a first control parameter based on at least the first feature, the first control parameter used for controlling delivery of atrial synchronous ventricular pacing.

Clause 27. The non-transitory computer readable medium of clause 26, wherein the instructions further cause the medical device to:
input the at least one cardiac electrical signal to a cardiac signal analyzer;
output a P-wave timing marker by the cardiac signal analyzer in response to an identified P-wave; and
determine that the first P-wave is in the diastolic period of the first cardiac cycle based on the P-wave timing marker output by the cardiac signal analyzer.

Clause 28. The non-transitory computer readable medium of any of clauses 26-27, wherein the instructions further cause the medical device to:
receive the cardiac motion signal and the at least one cardiac electrical signal sensed over a plurality of cardiac cycles of the first signal episode;
identify a plurality of P-waves including the first P-wave over the plurality of cardiac cycles;
identify at least one diastolic P-wave cycle among the plurality of cardiac cycles, each identified diastolic P-wave cycle being a cardiac cycle that is associated with a P-wave of the identified plurality of P-waves being during a diastolic period of the respective cardiac cycle;
determine a plurality of features of the motion signal comprising the first feature from the plurality of identified diastolic P-wave cycles; and
establish the first control parameter based on the plurality of features.

Clause 29. The non-transitory computer readable medium of any of clauses 26-28, wherein the instructions further cause the medical device to receive the motion signal over the plurality of cardiac cycles comprising asynchronous ventricular pacing pulses.

Clause 30. The non-transitory computer readable medium of any of clauses 26-29, wherein the instructions further cause the medical device to:
receive the cardiac motion signal and the at least one cardiac electrical signal sensed over a second signal episode during which atrial synchronous ventricular pacing is delivered according to the established first control parameter;
identify a second P-wave in the at least one cardiac electrical signal; determine that the second P-wave occurs in a diastolic period of a second cardiac cycle;
in response to the second P-wave being in the diastolic period of the second cardiac cycle, determine at least a second feature of the cardiac motion signal sensed during the second cardiac cycle; and
establish a second control parameter based on at least the second feature, the second control parameter used for controlling delivery of the atrial synchronous ventricular pacing.

Clause 31. The non-transitory computer readable medium of any of clauses 26-30, wherein the instructions further cause the medical device to establish the first control parameter by establishing an atrial event sensing control parameter used for sensing atrial events from the motion signal.

Clause 32. The non-transitory computer readable medium of clause 31, wherein the instructions further cause the medical device to:
determine the first feature of the cardiac motion signal sensed during the first cardiac cycle by determining a maximum amplitude of the motion signal during the diastolic period of the first cardiac cycle; and
establish the first control parameter based on at least the first feature by establishing a motion signal sensing vector for sensing the motion signal.

Clause 33. The non-transitory computer readable medium of clause 31, wherein the instructions further cause the medical device to:
determine the first feature of the cardiac motion signal sensed during the first cardiac cycle by determining a maximum amplitude of the motion signal during the diastolic period of the first cardiac cycle; and
establish the first control parameter based on at least the first feature by establishing an atrial event sensing threshold amplitude for sensing atrial events from the motion signal.

Clause 34. The non-transitory computer readable medium of clause 33, wherein the instructions further cause the medical device to:
determine that the first P-wave occurs in a late diastolic period of the first cardiac cycle; and
establish the first control parameter based on at least the first feature by establishing a late atrial event sensing threshold amplitude that is applied to the motion signal during an atrial event window for sensing atrial events from the motion signal.

Clause 35. The non-transitory computer readable medium of any of clauses 33-34, wherein the instructions further cause the medical device to:
identify a second P-wave in the at least one cardiac electrical signal; determine that the second P-wave occurs in an early diastolic period of a second cardiac cycle of the first signal episode;
in response to the second P-wave being in the early diastolic period of the second cardiac cycle, determine at least a second feature of the cardiac motion signal sensed during the second cardiac cycle; and
establish a second control parameter based on at least the second feature by establishing an early atrial event sensing threshold amplitude that is applied to the motion signal during a passive ventricular filling window for sensing atrial events from the motion signal.

Clause 36. The non-transitory computer readable medium of any of clauses 33-34, wherein the instructions further cause the medical device to:
determine that the first P-wave occurs in a late diastolic period of the first cardiac cycle;
determine a second feature of the cardiac motion signal sensed during the first cardiac cycle by determining a maximum amplitude of the motion signal during a passive ventricular filling window of the first cardiac cycle; and
establish a second control parameter based on at least the second feature by establishing an early atrial event sensing threshold amplitude that is applied to the motion signal during the passive ventricular filling window for sensing atrial events from the motion signal.

Clause 37. The non-transitory computer readable medium of clause 31, wherein the instructions further cause the medical device to:
set a test threshold amplitude;
determine the first feature of the cardiac motion signal sensed during the first cardiac cycle by determining a latest threshold crossing of the test threshold amplitude during a passive ventricular filling window of the first cardiac cycle; and
establish the first control parameter based on at least the first feature by establishing a passive ventricular filling window ending time used for sensing atrial events from the motion signal.

Clause 38. The non-transitory computer readable medium of clause 31, wherein the instructions further cause the medical device to:
determine the first feature of the cardiac motion signal sensed during the first cardiac cycle by determining a time of a maximum amplitude of the cardiac motion signal during the diastolic period of the first cardiac cycle; and
establish the first control parameter based on at least the first feature by establishing a passive ventricular filling window ending time used for sensing atrial events from the motion signal.

Clause 39. The non-transitory computer readable medium of clause 31, wherein the instructions further cause the medical device to:
determine the first feature of the cardiac motion signal sensed during the first cardiac cycle by determining a maximum amplitude of the motion signal during the first cardiac cycle; and
establish the first control parameter based on at least the first feature by establishing a post-ventricular atrial blanking period.

Clause 40. The non-transitory computer readable medium of any of clauses 26-39, wherein the instructions further cause the medical device to:
receive the cardiac motion signal and the at least one cardiac electrical signal sensed over a plurality of cardiac cycles of the first signal episode;
identify a plurality of P-waves over the plurality of cardiac cycles;
determine at least one PP interval between consecutively identified P-waves of the plurality of P-waves; and
establish a second control parameter based on the at least one PP interval for controlling delivery of atrial synchronous ventricular pacing.

Clause 41. The non-transitory computer readable medium of clause 40, wherein the instructions further cause the medical device to establish the second control parameter by establishing at least one of: a pacing lower rate, a rate smoothing increment, a post-ventricular atrial blanking period, and a maximum atrial tracking rate.

Clause 42. The non-transitory computer readable medium of any of clauses 26-41, wherein the instructions further cause the medical device to:
determine that a threshold percentage of atrioventricular synchronous pacing pulses is not met; and
in response to determining that the threshold percentage of atrioventricular synchronous pacing pulses is not met, adjust at least one atrial synchronous pacing control parameter.

Clause 43. The non-transitory computer readable medium of clause 42, wherein the instructions further cause the medical device to adjust the at least one atrial synchronous pacing control parameter by adjusting one of a plurality of control parameters that includes the first control parameter.

Clause 44. The non-transitory computer readable medium of any of clauses 42-43, wherein the instructions further cause the medical device to adjust the at least one control parameter by adjusting at least one of: a post-ventricular atrial blanking period, a passive ventricular filling window ending time, an early atrial event sensing threshold amplitude, or a late atrial event sensing threshold amplitude.

Clause 45. The non-transitory computer readable medium of any of clauses 42-44, wherein the instructions further cause the medical device to adjust the at least one control parameter by adjusting at least one of: a pacing lower rate, a rate smoothing increment, a maximum atrial tracking rate.

Clause 46. The non-transitory computer readable medium of any of clauses 26-45, wherein the instructions further cause the medical device to determine that the first P-wave occurs in a diastolic period of the first cardiac cycle of the first signal episode by:
applying a late diastolic threshold time; and
determining that the first P-wave occurs after the late diastolic threshold time and before a ventricular electrical event that ends the first cardiac cycle.

Clause 47. The non-transitory computer readable medium of any of clauses 26-46, wherein the instructions further cause the medical device to:
receive the cardiac motion signal and the at least one cardiac electrical signal sensed over a plurality of cardiac cycles of the first signal episode;
determine from the at least one cardiac electrical signal that less than a threshold number of the plurality of cardiac cycles are identified as diastolic P-wave cycles; and
adjust at least one asynchronous ventricular pacing interval in response to less than the threshold number of the plurality of cardiac cycles being identified as diastolic P-wave cycles.

Clause 48. The non-transitory computer readable medium of any of clauses 26-47, wherein the instructions further cause the medical device to:
determine a low confidence of P-wave identification in the at least one cardiac electrical signal; and
establish the first control parameter based on the cardiac motion signal sensed over a plurality of cardiac cycles.

Clause 49. The non-transitory computer readable medium of any of clauses 26-48, wherein the instructions further cause the medical device to transmit a programming command comprising the established first control parameter.

Clause 50. The non-transitory computer readable medium of any of clauses 26-48, wherein the instructions further cause the medical device to deliver atrial synchronous ventricular pacing according to the first control parameter.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a conceptual diagram illustrating a medical device system that may configured to sense cardiac electrical signals and motion signals induced by cardiac motion and/or flowing blood and provide pacing therapy to a patient's heart.

FIG. 2 is a conceptual diagram of the pacemaker shown in FIG. 1 according to one example.

FIG. 3 is a conceptual diagram of an example configuration of the pacemaker shown in FIG. 1.

FIG. 4 is an example of a motion sensor signal that may be sensed by a pacemaker motion sensor over a cardiac cycle.

FIG. 5 is an example of motion sensor signals acquired over two different cardiac cycles.

FIG. 6 is a flow chart of a method that may be performed by processing circuitry of a medical device system for establishing AVS pacing control parameters according to some examples.

FIG. 7 is a flow chart of a method that may be performed by processing circuitry of a medical device system for establishing control parameters used by a pacemaker during an AVS pacing mode according to another example.

FIG. 8 is a flow chart of a method that may be performed by processing circuitry of a medical device system for accumulating motion signal feature data for establishing AVS pacing control parameters according to another example.

FIG. 9 is a flow chart of a method that may be performed by processing circuitry of a medical device system for selecting a motion signal sensing vector according to one example.

FIG. 10 is a flow chart of a method that may be performed by processing circuitry of the medical device system of FIG. 1 for establishing control parameters used by a pacemaker during AVS pacing according to another example.

FIG. 11 is a flow chart of a method that may be performed by medical device system processing circuitry for fine tuning an ending time of a passive ventricular filling window according to some examples.

FIG. 12 is a flow chart of a method that may be performed by processing circuitry of a medical device system for establishing AVS pacing control parameters according to another example.

FIG. 13A and FIG. 13B depict a flow chart of a method that may be performed by processing circuitry of the medical device system of FIG. 1 according to another example.

FIG. 14 is a flow chart of a method that may be performed by the pacemaker of FIG. 1 according to some examples.

FIG. 15 is a diagram of cardiac signals in a signal episode that may be analyzed by processing circuitry of a medical device system for establishing AVS pacing control parameters according to some examples.

FIG. 16 is a flow chart of a method that may be performed by processing circuitry of medical device system for accumulating motion signal feature data for establishing AVS pacing control parameters according to another example.

FIG. 17 is a flow chart of a method for establishing AVS pacing control parameters according to some examples.

### DETAILED DESCRIPTION

In general, this disclosure describes techniques for establishing control parameters for use by an implantable medical device in sensing cardiac event signals from a cardiac mechanical signal and controlling ventricular pacing pulse delivery. As described below, atrial systolic events may be sensed from a signal produced by a sensor responsive to cardiac motion such that the motion signal can include an atrial event signal corresponding to atrial mechanical contraction during atrial systole and the active filling phase of the ventricle during ventricular diastole, sometimes referred to as the “atrial kick.” The medical device may sense atrial event signals from the motion signal according to atrial event sensing parameters. The atrial event sensing parameters may include a selected vector signal of the motion sensor, one or more sensing threshold amplitudes, and/or one or more time windows during which atrial event signals can be sensed. The techniques disclosed herein provide techniques for sensing atrial event signals from a motion sensor signal according to one or more atrial event sensing parameters by a ventricular pacemaker, which may be wholly implantable within or on a ventricular heart chamber, that has a motion sensor for producing a motion signal. In this way, atrial systolic events can be detected from a ventricular location for use in controlling atrial synchronous ventricular pacing, for example. Atrial synchronized ventricular pacing pulses can be delivered by a pacemaker implanted in the ventricle, for example, without requiring a sensor in or on the atria of the patient's heart for sensing atrial event signals for controlling delivery of AVS pacing pulses during an atrial synchronous ventricular pacing mode, also referred to herein as an “AVS pacing mode,” which can be denoted as a VDD pacing mode. During a VDD pacing mode, for example, ventricular pacing pulses are either inhibited (in response to sensing an intrinsic R-wave) or triggered (in response to sensing an atrial event signal) based on dual chamber (atrial and ventricular) sensing of cardiac event signals.

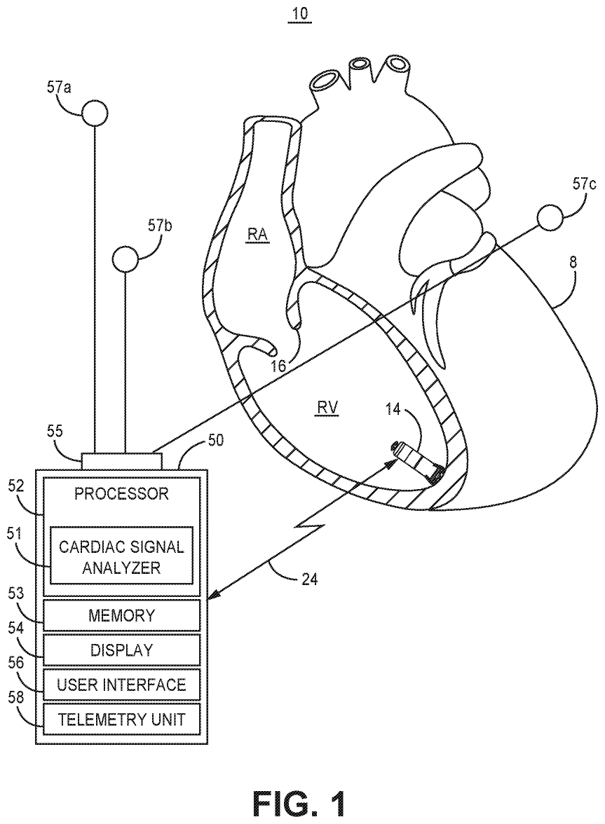

FIG. 1 is a conceptual diagram illustrating a medical device system 10 that may be configured to sense cardiac electrical signals and cardiac motion signals induced by cardiac motion and/or flowing blood and provide pacing therapy to a patient's heart 8. Medical device system 10 includes a ventricular pacemaker 14. Pacemaker 14 may be a leadless, transcatheter intracardiac pacemaker which is adapted for implantation wholly within a heart chamber, e.g., wholly within the right ventricle (RV) or wholly within the left ventricle (LV) of heart 8 for sensing cardiac signals and delivering ventricular pacing pulses. Pacemaker 14 may be reduced in size compared to subcutaneously implanted pacemakers and may be generally cylindrical in shape to enable transvenous implantation via a delivery catheter.

Pacemaker 14 is shown positioned in the RV, along an endocardial wall, e.g., near the RV apex though other locations are possible. The techniques disclosed herein are not limited to the pacemaker location shown in the example of FIG. 1 and other positions within or on heart 8 are possible. For example, pacemaker 14 may be positioned in the LV and configured to sense cardiac motion signals and deliver AVS pacing pulses to the LV using the techniques disclosed herein. Pacemaker 14 may be positioned within or on the RV or LV to provide respective right ventricular or left ventricular pacing and for sensing cardiac motion signals by a motion sensor from a ventricular location.

Pacemaker 14 is capable of producing electrical stimulation pulses, e.g., pacing pulses, delivered to heart 8 via one or more electrodes on the outer housing of the pacemaker. Pacemaker 14 is configured to deliver ventricular pacing pulses and sense a cardiac electrical signal using housing based electrodes for producing a ventricular electrogram (EGM) signal. The cardiac electrical signals may be sensed using the housing based electrodes that are also used to deliver pacing pulses to the RV.

Pacemaker 14 is configured to control the delivery of ventricular pacing pulses to the RV in a manner that promotes synchrony between atrial activation and ventricular activation, e.g., by setting an AV pacing interval in response to sensing atrial event signals for controlling the timing of delivered ventricular pacing pulses. That is, pacemaker 14 controls pacing pulse delivery to promote a desired AV delay between atrial contractions corresponding to atrial systole and ventricular pacing pulses delivered to cause ventricular myocardial depolarization and subsequent ventricular contraction.

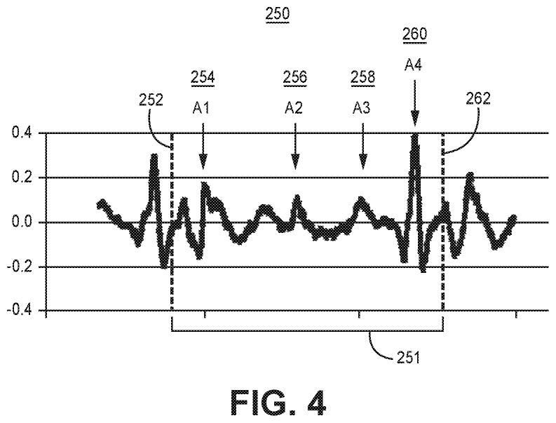

According to the techniques described herein, atrial systolic events producing the active ventricular filling phase are sensed by pacemaker 14 from a signal produced by a motion sensor such as an accelerometer enclosed by the housing of pacemaker 14. The motion signal produced by an accelerometer implanted within a ventricular chamber, which may be referred to as an “intraventricular motion signal,” includes motion signals caused by ventricular and atrial events. For example, acceleration of blood flowing into the RV through the tricuspid valve 16 between the RA and RV caused by atrial systole, and referred to as the “atrial kick,” may be detected by pacemaker 14 from the signal produced by an accelerometer included in pacemaker 14. Other motion signals that may be detected by pacemaker 14, such as motion caused by ventricular contraction and passive ventricular filling are described below in conjunction with FIG. 4.

Atrial P-waves that are attendant to atrial depolarization are relatively low amplitude signals in the near-field ventricular cardiac electrical signal received by pacemaker 14 (e.g., compared to the near-field R-wave) and therefore can be difficult to reliably detect from the cardiac electrical signal acquired by pacemaker 14 implanted in a ventricular location. Atrial-synchronized ventricular pacing by pacemaker 14 or other functions that rely on atrial sensing may not be reliable when based solely on a cardiac electrical signal received by pacemaker 14. According to the techniques disclosed herein, pacemaker 14 includes a mechanical cardiac signal sensor that produces a signal responsive to cardiac motion. In the illustrative examples disclosed herein, the sensor is a motion sensor such as an accelerometer enclosed by the housing of pacemaker 14. In other examples, the mechanical sensor may be a pressure sensor, a flow sensor, an acoustical sensor, an impedance sensor or other sensor responsive to cardiac motion. Atrial systolic event signals, also referred to herein as “atrial event signals,” attendant to atrial contraction during atrial systole, may be sensed from a variety of cardiac mechanical signals responsive to the motion of cardiac structures and/or flowing blood during a cardiac cycle including any of an acceleration signal, a pressure signal, an impedance signal, a blood flow signal, or a heart sound signal. In illustrative examples presented herein, a cardiac motion sensor included in pacemaker 14 is an accelerometer producing a motion signal from which pacemaker 14 is configured to sense an atrial event signal corresponding to atrial systole. Atrial event signals are sensed by pacemaker 14 according to atrial event sensing parameters that may be established or adjusted using the techniques disclosed herein.

Pacemaker 14 can be configured for bidirectional wireless communication with another medical device, which may be another implantable medical device and/or an external device. In the illustrative example of FIG. 1, pacemaker 14 is shown in communication with external device 50 via a wireless communication link 24. External device 50 may be used for programming operating control parameters uplinked to pacemaker 14, which may include various cardiac event sensing parameters and pacing control parameters utilized by pacemaker 14 for sensing cardiac event signals and controlling pacing pulse delivery. External device 50 may receive data downlinked from pacemaker 14 via communication link 24. The downlinked data may be patient-related data, cardiac signal data, delivered therapy data, device diagnostic data or the like.

Aspects of external device 50 may generally correspond to the external programming/monitoring unit disclosed in U.S. Pat. No. 5,507,782 (Kieval, et al.). External device 50 is often referred to as a “programmer” because it is typically used by a physician, technician, nurse, clinician or other qualified user for programming operating parameters in pacemaker 14. External device 50 may be located in a clinic, hospital or other medical facility. External device 50 may alternatively be embodied as a home monitor or a handheld device that may be used in a medical facility, in the patient's home, or another location.

External device 50 may include a processor 52, memory 53, display unit 54, user interface 56 and telemetry unit 58. Processor 52 controls external device operations and processes data and signals received from pacemaker 14 during a telemetry session via telemetry unit 58. Processor 52 may be configured to control telemetry unit 58 to transmit automatically determined and/or user-entered programming commands to pacemaker 14 and process data received from pacemaker 14 for display by display unit 54.

External device 50 may include external ports 55 for electrical connection to surface electrocardiogram (ECG) leads and electrodes 57a, 57b and 57c (collectively electrodes 57) that may be positioned cutaneously on a patient implanted with pacemaker 14. Processor 52 may receive ECG signals for display by display unit 54 for observation by a user during pacemaker implantation or during a patient follow-up. Observation of ECG signals may enable a user to confirm a patient's intrinsic rhythm and timing of R-waves, delivered ventricular pacing pulses, and/or atrial event signals sensed by pacemaker 14. While three ECG electrodes 57a, 57b and 57c are shown in FIG. 1 for the sake of illustration, external device 50 may be configured to receive ECG signals from one or more pairs of ECG electrodes. For example, processor 52 may be configured to receive multiple channels of ECG signals from multiple surface electrodes positioned on a patient relative to heart 8, e.g., three ECG channels, seven ECG channels, or twelve ECG channels.

As disclosed herein, at least one cardiac electrical signal, which may be an ECG signal or an EGM signal, is received by processing circuitry of medical device system 10. The cardiac electrical signal may be input to a cardiac signal analyzer 51, which may be a processing sub-unit or module of the processing circuitry of medical device system 10.

Cardiac signal analyzer 51 can be configured to identify P-wave signals in the cardiac electrical signal. The timing of identified P-wave signals, also referred to herein as “truthed P-waves,” in the cardiac electrical signal may be used by processing circuitry of medical device system 10 for accepting or rejecting a cardiac cycle from which motion signal data is obtained for use in establishing AVS pacing control parameters used by pacemaker 14 in delivering AVS pacing. The cardiac signal analyzer 51 may be implemented in processor 52 of external device 50, for instance.

In some examples, cardiac signal analyzer 51 may apply artificial intelligence (AI) techniques for analyzing the cardiac electrical signal for identifying P-waves attendant to atrial depolarization. Cardiac signal analyzer 51 may be, for example, a neural network model trained using AI techniques to receive at least one cardiac electrical signal input and output P-wave timing markers indicating the relative timing of P-waves identified in the input cardiac electrical signal. The cardiac signal input may be an ECG signal input received via interface 55. In other examples, external device 50 may be configured to receive an ECG signal transmitted wirelessly from another medical device via telemetry unit 58. For example, an ECG signal may be sensed by a wearable device such as a Holter monitor, home ECG monitor, smart watch, fitness tracker, or other ECG monitoring device. The ECG monitoring device may transmit an ECG signal to external device 50. In other examples, an ECG signal may be sensed by a second implantable medical device that is implanted in the patient in addition to pacemaker 14. Examples of other IMDs that may sense an ECG signal that may be transmitted to external device 50 include an implantable cardiac monitor such as the REVEAL LINQ™ Insertable Cardiac Monitor, available from Medtronic, Inc., Dublin, Ireland, or an implantable cardioverter defibrillator (ICD) coupled to transvenous or non-transvenous leads positioning electrodes outside of heart 8, e.g., in a suprasternal, substernal, transvenous extra-cardiac, or other extra-cardiac position for sensing ECG signals.

In other examples, cardiac signal analyzer 51 may be trained to receive an EGM signal input that is received from pacemaker 14 via telemetry unit 58. In other examples, cardiac signal analyzer 51 may receive an EGM signal input from a cardiac lead that is implanted temporarily in the patient's heart, e.g., within an atrial chamber. Cardiac signal analyzer 51 can receive multiple cardiac signal inputs, e.g., combined into multi-channel data points that may be aligned in time, for identifying P-waves in a cardiac electrical signal and outputting a P-wave timing marker that may be used by processing circuitry for rejecting or accepting cardiac cycles in obtaining motion signal data used to establish AVS pacing control parameters. The P-wave timing marker output by the cardiac signal analyzer 51 can be a signal, flag, sample time or other indicator of the time of an identified P-wave relative to a starting point (e.g., ventricular pacing pulse or R-wave) of the cardiac cycle during which the P-wave is identified.

Cardiac signal analyzer 51 may apply AI techniques for analyzing one or more input cardiac electrical signal(s) for identifying P-waves and output P-wave timing markers, which may include an associated level of confidence of each P-wave timing marker. The AI techniques may include deep learning such as convolutional neural networks (CNN), residual CNN, feed-forward neural network (FFNN), recurrent neural network (RNN), transformer, or other machine learning techniques such as decision tree, random forest model, or other machine learning approaches for establishing an AI model for identifying the presence (or absence) of P-waves in the cardiac electrical signal input with a relatively high level of confidence. Cardiac cycles for which the model confidence is low, which may be due to factors such as excessive signal noise or P-waves overlapping with the QRS waveforms or T-waves, may be flagged for downstream use. For example, processor 52 may exclude cardiac cycles or cardiac signal episodes or portions thereof having low level of confidence in P-wave identification from further analysis for use in establishing AVS pacing control parameters. Display unit 54 may display excluded cardiac cycles or cardiac signal episodes or portions thereof identified by processor 52 as being associated with a low level of confidence of identified P-waves and can be flagged as cardiac signal segments for facilitating troubleshooting a poor cardiac electrical signal quality (e.g., by repositioning surface electrodes, removing possible electrical noise sources from the area of the patient, etc.).

An AI model implemented in cardiac signal analyzer 51 may learn from cardiac electrical signal data obtained from a population of patients or an individual patient using machine learning. Cardiac signal analyzer 51 may be trained according to supervised or unsupervised algorithms using at least one cardiac electrical signal input, e.g., one or more ECG signals, one or more EGM signals or a combination of at least one ECG signal and at least one EGM signal. Cardiac signal analyzer 51 may be trained to also utilize ventricular event timing markers, e.g., intrinsic ventricular sensed events (e.g., R-waves and/or T-waves) and/or ventricular pace markers, which may be received from pacemaker 14. In some examples, cardiac signal analyzer 51 may identify R-waves and/or T-waves in addition to P-waves. An AI model implemented in cardiac signal analyzer 51 may be trained on a diverse development dataset of cardiac signal episodes and deployed in a locked state selected for optimized performance in identifying P-waves with a high level of confidence across a representative data cohort. The output of the cardiac signal analyzer 51 may be verified by an expert during training of the AI model.

Once trained on a development dataset, the AI model does not necessarily incorporate an aspect of continual learning or personalization such that the AI model of cardiac signal analyzer 51 can be a locked model. The cardiac signal analyzer 51 can be an AI model that, given a set of ECG and/or EGM inputs, along with possible ventricular event timing inputs identifies the relative location of P-waves within the signal episode (and within a given cardiac cycle). In some examples, cardiac signal analyzer 51 or processor 52 may determine that P-wave identifications are not confident enough to use for determining the timing of P-waves during cardiac cycles of a cardiac electrical signal episode and for use in establishing AVS pacing control parameters.

The output of cardiac signal analyzer 51 may include timing markers relative to the input signal(s) or as digital data indicating the timing of detected P-waves relative to a reference point. The reference point may be the beginning of a cardiac electrical signal episode input to cardiac signal analyzer 51, a ventricular pacing pulse marking the start of each one of multiple cardiac cycles in the cardiac electrical signal input and/or an R-wave marking the onset of a cardiac cycle as detected by cardiac signal analyzer from the cardiac electrical signal input or provided as a ventricular event timing marker from pacemaker 14, as examples.

Cardiac signal analyzer 51 is not limited to identifying P-waves based on an AI or machine learning model, however. In other examples, other cardiac electrical signal analysis techniques may be applied by cardiac signal analyzer 51 for identifying P-waves in a cardiac electrical signal and outputting a P-wave timing marker, with or without an associated confidence level, of identified P-waves. Aspects of some example systems and methods for identifying P-waves that may be implemented in cardiac signal analyzer 51 are generally disclosed in U.S. Application Publication No. 2009/0275850 (Mehendale et al.) and in U.S. Pat. No. 8,880,352 (Kale, et al.). Cardiac signal analyzer 51 may identify P-waves in the cardiac electrical signal by comparisons to a known P-wave template or one or more known P-wave templates or expected waveform features in other examples. Aspects of example P-wave identification methods utilizing a P-wave template that may be implemented in cardiac signal analyzer 51 are generally disclosed in U.S. Pat. No. 11,013,925 (Ghosh, et al). Cardiac signal analyzer 51 may be configured to determine various features or aspects of an input cardiac electrical signal, such as any combination of one or more amplitude(s), slope(s), polarity(ies), signal width(s), signal area(s) and/or P-wave template matching score(s), from time segments of the input cardiac signal(s) for identifying at least one P-wave location in the input cardiac electrical signal(s). It is to be understood that a variety of techniques may be conceived for identifying P-waves from a cardiac electrical signal that may be used in conjunction with the techniques disclosed herein for identifying cardiac cycles that include a P-wave during ventricular diastole. The term “cardiac cycle” as used herein can refer to one cycle of ventricular systole and ventricular diastole and may begin with a ventricular event, such as a ventricular pacing pulse or a ventricular R-wave, although it is recognized that one cardiac cycle may begin with any specified cyclical ventricular event or fiducial time point of the cardiac electrical signal.

Display unit 54, which may include a graphical user interface (GUI), displays data and other information to a user for reviewing IMD operation and programmed parameters and may display programmable parameters to a user for selection and programming of pacemaker 14. Display unit 54 may generate a display of a GUI presenting a visual representation of data and information relating to pacemaker functions to a user for reviewing pacemaker operation and programmed parameters as well as cardiac electrical signals, cardiac motion signals or other physiological data that may be acquired by pacemaker 14 and transmitted to external device 50 during an interrogation session.

Display unit 54 may be configured to display a GUI including various windows, icons, user selectable menus, etc. to facilitate interaction by a user with the external device 50 and pacemaker 14. Display unit 54 may function as an input and/or output device using technologies including liquid crystal displays (LCD), quantum dot display, dot matrix displays, light emitting diode (LED) displays, organic light-emitting diode (OLED) displays, cathode ray tube displays, e-ink, or monochrome, color, or any other type of display capable of generating tactile, audio, and/or visual output. In some examples, display unit 54 is a presence-sensitive display that may serve as a user interface device that operates both as one or more input devices and one or more output devices. Display unit 54 may be configured to present representations of cardiac signals and/or data derived therefrom used for selecting AVS pacing control parameters according to the techniques disclosed herein.

Such representations reduce the burden on a user or clinician in interpreting cardiac electrical signals and simplify programming of pacemaker 14 in a patient-specific manner for promoting proper timing of ventricular pacing pulses relative to atrial event signals during AVS pacing. Confirming P-waves in an ECG or EGM signal based on human observation of cardiac signals can be challenging and requires considerable user expertise. Confirmation of P-waves, however, can improve the selection of cardiac cycles that are used in establishing or adjusting AVS pacing control parameters. The AVS pacing control parameters may be used in sensing atrial event signals from a motion signal, for example. As further described below, confirmed P-wave timing during the ventricular diastolic phase of a cardiac cycle can be used for accepting a given cardiac cycle for use in acquiring motion signal data that is used in establishing AVS pacing control parameters.

Accordingly, the techniques set forth herein for establishing AVS pacing control parameters provide specific improvements to the computer-related field of programming medical devices and reporting medical device-related information and data that have practical applications. For example, the use of the techniques herein may enable processing circuitry of a medical device system to establish AVS pacing control parameters during an automatic setup procedure and generate visualizations of cardiac electrical signal and/or motion signal data, that may be annotated with confirmed P-wave timing markers relative to a cardiac electrical signal and/or motion signal. In some examples, after establishing AVS pacing control parameters, cardiac signal data may be displayed by display unit 54 annotated with confirmed P-wave timing markers relative to atrial event signals sensed by pacemaker 14 from a motion signal according to established atrial event sensing parameters.

Such visual representations may more accurately inform a clinician or user as to how pacemaker 14 is expected to perform in delivering AVS pacing. The setup procedures disclosed herein for establishing AVS pacing control parameters using P-wave timing markers output by a cardiac signal analyzer can reduce the likelihood of human error in programming pacemaker operating parameters. Furthermore, the techniques disclosed herein may reduce the complexity of programming pacemaker 14. The process of manually selecting and programming AVS pacing control parameters, which may include multiple atrial event sensing control parameters and multiple ventricular pacing control parameters, can be challenging and require a high level of expertise in interpreting cardiac signals. The AVS pacing control parameters affect the performance of pacemaker 14 in delivering a relatively high percentage of AVS pacing pulses out of all ventricular events (e.g., compared to a relative percentage of non-atrial synchronized ventricular pacing pulses).

For example, ventricular events that may begin a cardiac cycle can include AVS pacing pulses, non-AVS pacing pulses (also referred to as asynchronous ventricular pacing pulses) or an intrinsic R-wave that is sensed before a pacing interval expires. Non-AVS pacing pulses may be delivered at a lower rate interval (LRI) corresponding to a programmed lower ventricular rate (sometimes referred to as a “base pacing rate”), a rate smoothing interval (RSI) or other pacing interval that is not an AV pacing interval that synchronizes the ventricular pacing pulse to an atrial event signal. In a patient having AV block, a high percentage of AVS pacing pulses that are correctly tracking true atrial event signals is desired to promote heart chamber synchrony and the associated hemodynamic benefits. However, selecting the optimal AVS pacing control parameters for achieving a high percentage of cardiac cycles that start with AVS pacing pulses out of all cardiac cycles can be a time-consuming task that poses significant burden on a clinician. The techniques disclosed herein can be implemented in a medical device system for establishing control parameters used by a pacemaker configured to deliver AVS pacing in a manner that reduces the expertise and time required by a clinician in programming AVS pacing control parameters. As such, the techniques disclosed herein may enable a medical device, such as pacemaker 14, to be programmed to sense atrial event signals and deliver AVS pacing pulses in a manner that is simplified, flexible, and patient-specific and achieves effective AVS pacing based on a relatively high percentage of AVS pacing cycles out of all cardiac cycles in a given time period.

User interface 56 may include a mouse, touch screen, keypad or the like to enable a user to interact with external device 50 to initiate a telemetry session with pacemaker 14 for retrieving data from and/or transmitting data to pacemaker 14. Telemetry unit 58 includes a transceiver and antenna configured for bidirectional communication with a telemetry circuit included in pacemaker 14 and is configured to operate in conjunction with processor 52 for sending and receiving data relating to pacemaker functions via communication link 24, which may include data relating to ventricular pacing and atrial event signal sensing.

Telemetry unit 58 includes a transceiver and antenna configured for bidirectional communication with a telemetry circuit included in pacemaker 14 and can be configured to operate in conjunction with processor 52 for sending and receiving data relating to pacemaker functions via communication link 24. Communication link 24 may be established between pacemaker 14 and external device 50 using a wireless radio frequency (RF) link such as BLUETOOTH®, Wi-Fi, or Medical Implant Communication Service (MICS) or other RF or communication frequency bandwidth or communication protocols. In some examples, external device 50 may include a programming head that is placed proximate pacemaker 14 to establish and maintain a communication link 24, and in other examples external device 50 and pacemaker 14 may be configured to communicate using a distance telemetry algorithm and circuitry that does not require the use of a programming head and does not require user intervention to maintain a communication link. An example RF telemetry communication system that may be implemented in system 10 is generally disclosed in U.S. Pat. No. 5,683,432 (Goedeke, et al.). Data stored or acquired by pacemaker 14, including EGM signals or associated data derived therefrom, motion signals or associated data derived therefrom, results of device diagnostics, and histories of sensed R-waves, atrial event signals and/or delivered ventricular pacing pulses or other data may be retrieved from pacemaker 14 by external device 50 following an interrogation command transmitted by telemetry unit 58.

External device 50 may be implemented in one of a number of computing systems configured to receive a cardiac electrical signal and a cardiac motion signal for establishing AVS pacing control parameters. External device 50 may be a personal computer, a medical device programmer, a home monitor, a wearable patient monitor, mobile device such as a smart phone, laptop, tablet, personal digital assistant or the like. In some examples, external device 50 is a computing device of a remote patient monitoring system such as a CARELINK™ monitor available from Medtronic, Inc., Dublin, Ireland.

At the time of implant, during patient follow-up visits, or any time after pacemaker implantation, processing circuitry of pacemaker 14 and/or external device 50 may perform a set-up procedure to establish parameters used in sensing atrial event signals from the motion sensor signal. In some examples, a user may initiate the automatic set up process by entering a “one-click” command. During the automatic setup process cardiac signals can be received or acquired by external device 50 and/or pacemaker 14 and processed and analyzed by processing circuitry for establishing one or more AVS pacing control parameter settings to be used by pacemaker 14.

The patient may be standing, sitting, lying down or ambulatory during the process. The setup procedure may include acquiring motion sensor signal episodes and determining motion signal features for establishing control parameters used by pacemaker 14 during AVS pacing. Motion sensor signal data may be transmitted to external device 50 for processing and analysis by external device processor 52 and, in some examples, for display on display unit 54 in the form of motion signal episodes, histograms or other representations of motion signal features, representative values or tabulations of motion signal features, or other visual representations of motion signal data. The AVS pacing control parameters established based on the motion sensor signal data according to the techniques disclosed herein may be applied by pacemaker 14 in response to a command transmitted by external device 50. The AVS pacing control parameters put applied by pacemaker 14 may be from external device 50 and may be presented in a display on display unit 54, allowing a clinician to review and accept or modify the established control parameters, e.g., using user interface 56.

It is contemplated that external device 50 may be in wired or wireless connection to a communications network via telemetry unit 58 that includes a transceiver and antenna or via a hardwired communication line for transferring data to a centralized database or computer to allow remote management of the patient. Remote patient management systems including a centralized patient database may be configured to utilize the presently disclosed techniques to enable a clinician to review cardiac electrical signals, the motion sensor signal, and/or marker channel data and authorize programming of sensing and therapy control parameters in pacemaker 14, e.g., after viewing a visual representation of ECG and/or EGM signals, motion sensor signal and marker channel data. One example of a remote patient management system in which the currently disclosed techniques may be implemented at least in part is the CARELINK™ Network (Medtronic, Inc. Dublin, Ireland).

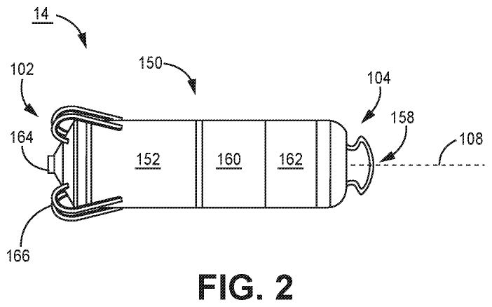

FIG. 2 is a conceptual diagram of pacemaker 14 shown in FIG. 1 according to one example. Pacemaker 14 includes electrodes 162 and 164 spaced apart along the housing 150 of pacemaker 14 for sensing cardiac electrical signals and delivering pacing pulses. Electrode 164 is shown as a tip electrode extending from a distal end 102 of pacemaker 14, and electrode 162 is shown as a ring electrode along a mid-portion of housing 150, for example adjacent proximal end 104. Distal end 102 is referred to as “distal” in that it is expected to be the leading end as pacemaker 14 is advanced through a delivery tool, such as a catheter, and placed against a targeted pacing site.

Electrodes 162 and 164 form an anode and cathode pair for bipolar cardiac pacing and sensing. In alternative embodiments, pacemaker 14 may include two or more ring electrodes, two tip electrodes, and/or other types of electrodes exposed along pacemaker housing 150 for delivering electrical stimulation to heart 8 and sensing cardiac electrical signals. Electrodes 162 and 164 may be, without limitation, titanium, platinum, iridium or alloys thereof and may include a low polarizing coating, such as titanium nitride, iridium oxide, ruthenium oxide, platinum black, among others. Electrodes 162 and 164 may be positioned at locations along pacemaker 14 other than the locations shown.

Tip electrode 162 is shown as a relatively flat button electrode but could be a hemispherical electrode or other non-tissue piercing electrode in other examples. In still other examples, tip electrode 162 may be formed as a tissue-piercing electrode, e.g., a helical screw-in electrode, a fishhook electrode, or a straight shaft with a tissue-piercing distal tip. A distal portion of a tissue-piercing electrode may form the active electrode portion of the electrode positioned in cardiac tissue at a ventricular pacing site. For example, distal tip electrode 162 may be advanceable into the inter-ventricular septum of a patient's heart to deliver ventricular pacing to septal tissue which may include ventricular myocardial tissue and/or a portion of the His-Purkinje conduction system of the heart. For instance, a distal tip electrode of pacemaker 14 may be advanced in a left portion of the septum in the area of the left bundle branch of the heart's native conduction system. Examples of leadless intracardiac pacemakers that may be configured for delivering cardiac pacing pulses to the His-Purkinje conduction system that may be used in conjunction with the techniques described herein are generally disclosed in U.S. Publication No. 2019/0111270 (Zhou) and U.S. Publication No. 2019/0083800 (Yang, et al.).

Housing 150 is formed from a biocompatible material, such as a stainless steel or titanium alloy. In some examples, the housing 150 may include an insulating coating.

Examples of insulating coatings include parylene, urethane, PEEK, or polyimide, among others. The entirety of the housing 150 may be insulated, but only electrodes 162 and 164 uninsulated. Electrode 164 may serve as a cathode electrode and be coupled to internal circuitry, e.g., a pacing pulse generator and cardiac electrical signal sensing circuitry, enclosed by housing 150 via an electrical feedthrough crossing housing 150. Electrode 162 may be formed as a conductive portion of housing 150 defining a ring electrode that is electrically isolated from the other portions of the housing 150 as generally shown in FIG. 2. In other examples, the entire periphery of the housing 150 may function as an electrode that is electrically isolated from tip electrode 164, instead of providing a localized ring electrode such as anode electrode 162. Electrode 162 formed along an electrically conductive portion of housing 150 may serve as a return anode during pacing and sensing.

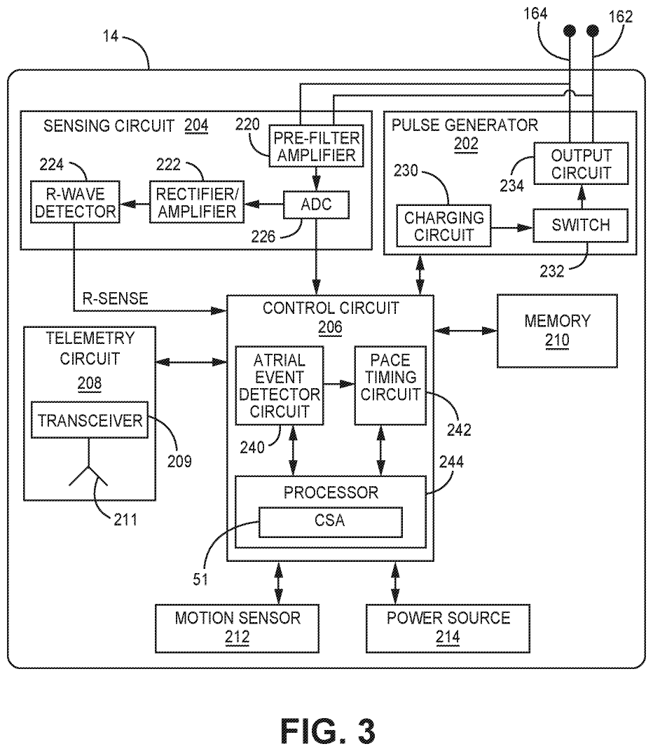

The housing 150 includes a control electronics subassembly 152, which houses the electronics for sensing cardiac signals, producing pacing pulses and controlling therapy delivery and other functions of pacemaker 14 as described below in conjunction with FIG. 3. A motion sensor may be implemented as an accelerometer enclosed within housing 150 in some examples. The accelerometer provides a signal to a processor included in control electronics subassembly 152 for signal processing and analysis for detecting atrial systolic event signals, e.g., for use in controlling the timing of ventricular pacing pulses.

The accelerometer may be a three-dimensional accelerometer. In some examples, the accelerometer may have one “longitudinal” axis that is parallel to or aligned with the longitudinal axis 108 of pacemaker 14 and two orthogonal axes that extend in radial directions relative to the longitudinal axis 108. Practice of the techniques disclosed herein, however, are not limited to a particular orientation of the accelerometer within or along housing 150. In other examples, a one-dimensional accelerometer may be used to obtain a cardiac motion signal from which atrial systolic event signals are sensed. In still other examples, a two dimensional accelerometer or other multi-dimensional accelerometer may be used. Each axis of a single or multi-dimensional accelerometer may be defined by a piezoelectric element, micro-electrical mechanical system (MEMS) device or other sensor element capable of producing an electrical signal in response to changes in acceleration imparted on the sensor element, e.g., by converting the acceleration to a force or displacement that is converted to the electrical signal. In a multi-dimensional accelerometer, the sensor elements may be arranged orthogonally with each sensor element axis orthogonal relative to the other sensor element axes. Orthogonal arrangement of the elements of a multi-axis accelerometer, however, is not necessarily required.

Each sensor element may produce an acceleration signal corresponding to a vector aligned with the axis of the sensor element. As described below, techniques disclosed herein may include selecting a vector signal of a multi-dimensional accelerometer (also referred to as a “multi-axis” accelerometer) for use in sensing atrial systolic event signals. In some cases one, two or all three axis signals produced by a three dimensional accelerometer may be selected as a vector signal for use in detecting atrial systolic events, e.g., for controlling atrial synchronous ventricular pacing delivered by pacemaker 14. Techniques disclosed herein for establishing AVS pacing control parameters may include selecting a motion signal vector for sensing atrial event signals based on analysis of features of the motion signal determined from cardiac cycles that are accepted based on P-wave timing during the ventricular diastolic phase of the cardiac cycles. As used herein, the terms “diastolic period,” “ventricular diastole” and “the ventricular diastolic phase” generally refers to the portion of the cardiac cycle during which ventricular relaxation and filling occurs, which can generally commence with closure of the aortic and pulmonary valves and ends when the next ventricular electrical event occurs or the onset of the subsequent ventricular contraction and ejection of blood from the ventricles (generally known as the “systolic period,” “ventricular systole” or the “ventricular systolic phase”). The P-wave timing during a cardiac cycle may be determined based on the output of cardiac signal analyzer 51. The P-wave timing may be determined during an asynchronous ventricular pacing mode such that, during a given cardiac cycle, the P-wave may occur at any time (or not at all) during the cardiac cycle that begins with a non-AVS ventricular pacing pulse.

Housing 150 further includes a battery subassembly 160, which provides power to the control electronics subassembly 152. Battery subassembly 160 may include features of the batteries disclosed in commonly-assigned U.S. Pat. No. 8,433,409 (Johnson, et al.) and U.S. Pat. No. 8,541,131 (Lund, et al.).

Pacemaker 14 may include a set of fixation tines 166 to secure pacemaker 14 to patient tissue, e.g., by actively engaging with the ventricular endocardium and/or interacting with the ventricular trabeculae. Fixation tines 166 are configured to anchor pacemaker 14 to position electrode 164 in operative proximity to a targeted tissue for delivering therapeutic electrical stimulation pulses. Numerous types of active and/or passive fixation members may be employed for anchoring or stabilizing pacemaker 14 in an implant position. Pacemaker 14 may include a set of fixation tines as disclosed in commonly-assigned U.S. Pat. No. 9,775,872 (Grubac, et al.).

Pacemaker 14 may optionally include a delivery tool interface 158. Delivery tool interface 158 may be located at the proximal end 104 of pacemaker 14 and can be configured to connect to a delivery device, such as a catheter, used to position pacemaker 14 at an implant location during an implantation procedure, for example within a heart chamber.

FIG. 3 is a conceptual diagram of an example configuration of pacemaker 14 shown in FIG. 1. Pacemaker 14 includes a pulse generator 202, a cardiac electrical signal sensing circuit 204 (also referred to herein as “sensing circuit” 204), a control circuit 206, memory 210, telemetry circuit 208, motion sensor 212 and a power source 214. The various circuits represented in FIG. 3 may be combined on one or more integrated circuit boards which include a specific integrated circuit (ASIC), an electronic circuit, a processor (shared, dedicated, or group) and memory that execute one or more software or firmware programs, a combinational logic circuit, state machine or other suitable components that provide the described functionality.

Motion sensor 212 may include an accelerometer in the examples described herein. Motion sensor 212 is not limited to being an accelerometer, however, and other motion sensors may be utilized successfully in pacemaker 14 for sensing cardiac motion signals or any cardiac mechanical signal correlated to cardiac motion according to the techniques described herein. As indicated above, motion sensor 212 may include a multi-axis sensor, e.g., a two-dimensional or three-dimensional sensor, with each axis providing an axis signal that may be analyzed individually or in combination for sensing atrial event signals. Motion sensor 212 produces an electrical signal correlated to motion or vibration of sensor 212 (and pacemaker 14), e.g., when subjected to flowing blood and cardiac motion. The motion sensor 212 may include one or more filter, amplifier, rectifier, analog-to-digital converter (ADC) and/or other components for producing a motion signal that is passed to control circuit 206. For example, each axis signal produced by each individual axis of a multi-axis accelerometer may be filtered by a high pass filter, e.g., a 10 Hz high pass filter. The filtered signal may be digitized by an ADC and rectified for use by atrial event detector circuit 240, alone or in combination with one or more other individual axis signals, for detecting atrial systolic events. The high pass filter may be lowered (e.g., to 5 Hz) if needed to sense atrial event signals that have lower frequency content. In some examples, high pass filtering is performed with no low pass filtering. In other examples, each accelerometer axis signal is filtered by a low pass filter, e.g., a 30 Hz low pass filter, with or without high pass filtering.

One example of an accelerometer for use in implantable medical devices that may be implemented in conjunction with the techniques disclosed herein is generally disclosed in U.S. Pat. No. 5,885,471 (Ruben, et al.). An implantable medical device arrangement including a piezoelectric accelerometer for detecting patient motion is disclosed, for example, in U.S. Pat. No. 4,485,813 (Anderson, et al.) and U.S. Pat. No. 5,052,388 (Sivula, et al.). Examples of three-dimensional accelerometers that may be implemented in pacemaker 14 and used for sensing atrial event signals using the presently disclosed techniques are generally described in U.S. Pat. No. 5,593,431 (Sheldon) and U.S. Pat. No. 6,044,297 (Sheldon). Other accelerometer designs may be used for producing an electrical signal that is correlated to motion imparted on pacemaker 14 due to ventricular and atrial events.

During an auto-setup procedure, control circuit 206 may receive the motion signal from accelerometer 212 for transmission to external device 52 via telemetry circuit 208 for processing and analysis according to the techniques disclosed herein for establishing AVS pacing control parameters. The motion signal sensed by accelerometer 212 may be filtered, amplified, and rectified and buffered in memory 210 as a digitally sampled signal for transmission to external device 52. The motion signal and a contemporaneously sensed cardiac electrical signal, e.g., an ECG or EGM signal, may be processed and analyzed by external device processor 52 as described below for determining motion signal features from cardiac cycles that are accepted based on output of P-wave timing markers from cardiac signal analyzer 51 (shown in FIG. 1). In some examples, the motion signal is transmitted in real time via telemetry circuit 208 to enable transmission of a relatively high sampling rate (e.g., 128 to 512 Hz), high fidelity signal from which motion signal data is determined by processing circuit 52 for use in establishing AVS pacing control parameters.

In other examples, control circuit 206 may determine motion signal data from the motion signal (received from one or more axes of the motion sensor) for each cardiac cycle. Motion signal data that is determined from cardiac cycles that are rejected based on the output of cardiac signal analyzer 51 may be subsequently rejected and not used for establishing AVS pacing control parameters. In this case, control circuit 206 may receive P-wave timing marker signals or cardiac cycle rejection data from external device 50 for use in determining which motion signal data is associated with accepted cardiac cycles and which motion signal data is associated with rejected cardiac cycles. The motion signal data determined from accepted cardiac cycles can be used by control circuit 206 for determining AVS pacing control parameters. The motion signal data from rejected cardiac cycles may be discarded or ignored.

In still other examples, motion signal data may be determined by control circuit 206 from each of multiple cardiac cycles during a data collection time period. The motion signal data may be transmitted to external device 50 including timing or cardiac cycle number information so that external device processor 52 may determine which motion signal data to reject and which motion signal data to accept based on the output of cardiac signal analyzer 51. When a P-wave timing marker output from cardiac signal analyzer 51 indicates a P-wave during ventricular diastole, e.g., a late diastole P-wave, the motion signal data associated with that cardiac cycle is accepted. When the P-wave timing marker output from cardiac signal analyzer 51 indicates that a P-wave does not occur during ventricular diastole, or in some cases during a late portion of ventricular diastole, the motion signal data associated with that cardiac cycle can be rejected in some examples.

Sensing circuit 204 is configured to receive a cardiac electrical signal via electrodes 162 and 164 by a pre-filter and amplifier circuit 220. Pre-filter and amplifier circuit may include a high pass filter to remove DC offset, e.g., a 2.5 to 5 Hz high pass filter, or a wideband filter having a passband of 2.5 Hz to 100 Hz to remove DC offset and high frequency noise. Pre-filter and amplifier circuit 220 may further include an amplifier to amplify the “raw” cardiac electrical signal passed to analog-to-digital converter (ADC) 226. ADC 226 may pass a multi-bit, digital electrogram (EGM) signal to control circuit 206 for use by atrial event detector circuit 240 in identifying ventricular electrical events (e.g., R-waves or T-waves) and/or atrial electrical events, e.g., P-waves. Identification of cardiac electrical events may be used in algorithms for establishing atrial sensing control parameters and for sensing atrial systolic events from the motion sensor signal. The EGM signal received from sensing circuit 204 may be transmitted in real time to external device 50 during an auto-setup procedure with a contemporaneous motion signal from motion sensor 212 for processing and analysis by external device processor 52 in some examples. In other examples, episodes of the EGM signal may be stored in memory 210 and transmitted to external device 50.

The digital signal from ADC 226 may be passed to rectifier and amplifier circuit 222 of sensing circuit 204, which may include a rectifier, bandpass filter, and amplifier for passing a cardiac signal to R-wave detector 224. R-wave detector 224 may include a sense amplifier or other detection circuitry that compares the incoming rectified, cardiac electrical signal to an R-wave sensing threshold, which may be an auto-adjusting threshold. When the incoming signal crosses the R-wave sensing threshold, the R-wave detector 224 produces an R-wave sensed event signal (R-sense) that is passed to control circuit 206. In other examples, R-wave detector 224 may receive the digital output of ADC 226 for detecting R-waves by a comparator, morphological signal analysis of the digital EGM signal or other R-wave detection techniques.

Control circuit 206 may include an atrial event detector circuit 240, pace timing circuit 242, and processor 244. Processor 244 may provide sensing control signals to sensing circuit 204, e.g., R-wave sensing threshold, sensitivity, and various blanking and refractory periods applied to the cardiac electrical signal for controlling R-wave sensing. Control circuit 206 may receive R-wave sensed event signals and/or digital cardiac electrical signals from sensing circuit 204 for use in detecting and confirming cardiac events and controlling ventricular pacing. For example, R-wave sensed event signals passed from R-wave detector 224 to control circuit 206 may be used for inhibiting a scheduled ventricular pacing pulse and restarting a ventricular pacing interval by pace timing circuit 242. The R-wave sensed event signals may be used by atrial event detector circuit 240 for setting a post-ventricular atrial blanking period, a post-ventricular atrial refractory period, and/or one or more atrial event sensing windows during which atrial event sensing threshold(s) are applied for use in sensing atrial systolic events from a motion signal received from motion sensor 212.

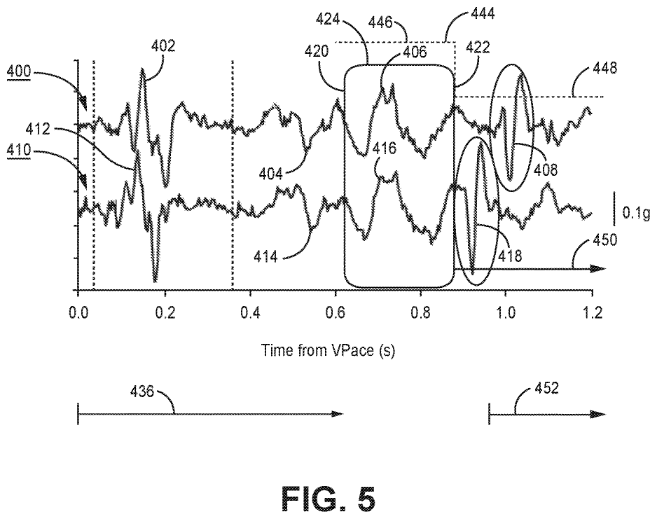

Atrial event detector circuit 240 is configured to detect atrial systolic events from a signal received from motion sensor 212. Techniques for setting time windows and atrial event sensing threshold amplitude used in sensing atrial event signals are described below, e.g., in conjunction with FIG. 5. Atrial event detector circuit 240 receives a motion signal from motion sensor 212 and may start a post-ventricular atrial blanking period (PVABP) in response to a ventricular electrical event, e.g., an R-wave sensed event signal from sensing circuit 204 or delivery of a ventricular pacing pulse by pulse generator 202. The PVABP may correspond to a time period after the ventricular electrical event during which ventricular mechanical events, e.g., corresponding to ventricular contraction are expected to occur. When ventricular pacing is properly synchronized to atrial events, an atrial event is not expected to occur during the atrial blanking period, corresponding to ventricular systole. The motion signal peaks that occur during the PVABP, therefore, are not sensed as atrial events. The PVABP may be applied by atrial event detector circuit 240 to define a time period following a ventricular electrical event during which an atrial systolic event is not sensed. The motion sensor signal, however, is not necessarily blanked or not sensed during this time period. The motion sensor 212 may still sense the motion signal, and control circuit 206 may still receive the motion sensor signal during all or a portion of the PVABP. Control circuit 206 may analyze the motion signal sensed during the PVABP for purposes other than sensing the atrial event signal.

Atrial event detector circuit 240 determines if the motion sensor signal satisfies atrial systolic event sensing criteria outside of the PVABP. As described below, atrial event detector circuit 240 may set time windows corresponding to the passive ventricular filling phase and the active ventricular filling phase during a cardiac cycle following a ventricular electrical event, either an R-wave sensed event signal from sensing circuit 204 or a ventricular pacing pulse delivered by pulse generator 202. The earliest crossing of the atrial event sensing threshold by the motion signal during one of these windows may be sensed as the atrial event signal. As described below, two different atrial event sensing threshold values may be established for applying during the passive filling phase window and after the passive filling phase window (during an active filling phase window also referred to below as an “A4 window”).

Atrial event detector circuit 240 may pass an atrial event detection signal to processor 244 and/or pace timing circuit 242 in response to sensing an atrial event signal. Pace timing circuit 242 (or processor 244) may additionally receive R-wave sensed event signals from R-wave detector 224 for use in controlling the timing of ventricular pacing pulses delivered by pulse generator 202. Processor 244 may include one or more clocks for generating clock signals that are used by pace timing circuit 242 to time out an AV pacing interval that is started upon receipt of an atrial event detection signal from atrial event detector circuit 240. Pace timing circuit 242 may include one or more pacing escape interval timers or counters that are used to time out the AV pacing interval, which may be a programmable interval stored in memory 210 and retrieved by processor 244 for use in setting the AV pacing interval used by pace timing circuit 242. The AV pacing interval may be between 10 and 100 ms, as examples. One application of atrial sensed event signals produced by atrial event detector circuit 240 is for setting AV pacing intervals for controlling the timing of ventricular pacing pulses. Control circuit 206, however, may use atrial sensed event signals for other purposes in addition to starting AV pacing intervals or instead of starting AV pacing intervals, e.g., when pacemaker 14 is not operating in an AVS pacing mode.

Pace timing circuit 242 may additionally include an LRI timer for controlling a minimum ventricular pacing rate. For example, if an atrial systolic event is not sensed from the motion signal for triggering a ventricular pacing pulse at the AV pacing interval, a ventricular pacing pulse may be delivered by pulse generator 202 upon expiration of the LRI to prevent ventricular asystole and maintain a minimum ventricular rate. In order to avoid an abrupt change in ventricular rate, the LRI timer may be set to a rate smoothing interval (RSI) that is gradually adjusted toward the LRI from a current ventricular rate.

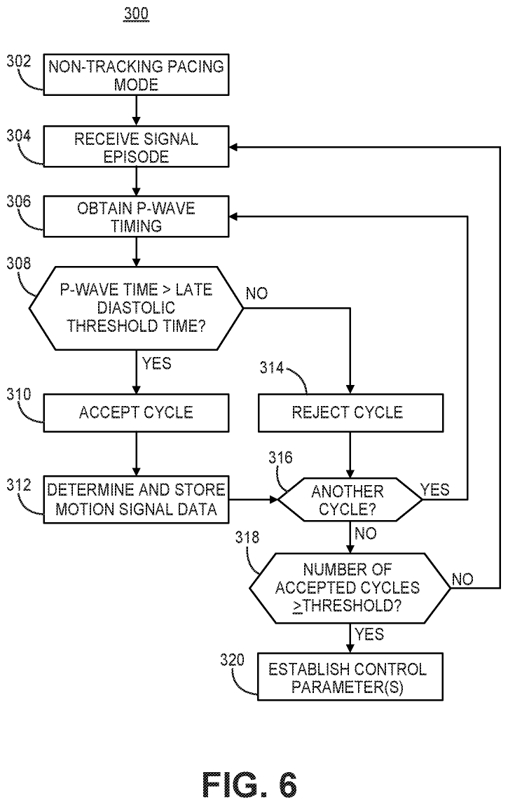

At times, control circuit 206 may control pulse generator 202 to deliver ventricular pacing in a non-atrial tracking ventricular pacing mode (also referred to herein as “asynchronous ventricular pacing”) during a process for establishing AVS pacing control parameters. The asynchronous ventricular pacing mode may be denoted as a VDI pacing mode in which ventricular pacing pulses are delivered in the absence of a sensed R-wave and inhibited in response to an R-wave sensed event signal from sensing circuit 204. Dual chamber sensing (e.g., R-wave sensing by sensing circuit 204 and atrial event signal sensing from the motion signal by atrial event detector circuit 240) may be performed during the asynchronous ventricular pacing mode. As described below, e.g., in conjunction with FIGS. 6-14, AVS pacing control parameters established during a VDI pacing mode may include an atrial event sensing vector of the motion sensor producing a signal from which the atrial event signals are sensed, the end of a passive ventricular filling window, the atrial event sensing threshold amplitude values applied during and after the passive ventricular filling window, and a rate smoothing interval increment.

In some examples, cardiac signal analyzer (CSA) 51 may be implemented in processor 244 of control circuit 206 for identifying P-waves from the EGM signal received from sensing circuit 204. Cardiac signal analyzer 51 may output timing markers corresponding to the timing of identified P-waves in the EGM signal. Processor 244 may accept or reject a cardiac cycle based on the timing of identified P-waves during cardiac cycles starting with a non-AVS pacing pulse, e.g., during a VDI pacing mode. The motion signal received from motion sensor 212 during one or more accepted cardiac cycles may be analyzed by processor 244 according to the techniques disclosed herein for establishing or adjusting AVS pacing control parameters.

Pulse generator 202 generates electrical pacing pulses that are delivered to the ventricles of the patient's heart via cathode electrode 164 and return anode electrode 162. In addition to providing control signals to pace timing circuit 242 and pulse generator 202 for controlling the timing of ventricular pacing pulses, processor 244 may retrieve programmable pacing control parameters, such as pacing pulse amplitude and pacing pulse width, which are passed to pulse generator 202 for controlling pacing pulse delivery. Pulse generator 202 may include charging circuit 230, switching circuit 232 and an output circuit 234.

Charging circuit 230 may include a holding capacitor that may be charged to a pacing pulse amplitude by a multiple of the battery voltage signal of power source 214 under the control of a voltage regulator. The pacing pulse amplitude may be set based on a control signal from control circuit 206. Switching circuit 232 may control when the holding capacitor of charging circuit 230 is coupled to the output circuit 234 for delivering the pacing pulse. For example, switching circuit 232 may include a switch that is activated by a timing signal received from pace timing circuit 242 upon expiration of an AV pacing interval (or LRI) and kept closed for a programmed pacing pulse width to enable discharging of the holding capacitor of charging circuit 230. The holding capacitor, previously charged to the pacing pulse voltage amplitude, is discharged across electrodes 162 and 164 through the output capacitor of output circuit 234 for the programmed pacing pulse duration. Examples of pacing circuitry generally disclosed in U.S. Pat. No. 5,507,782 (Kieval, et al.) and in U.S. Pat. No. 8,532,785 (Crutchfield, et al) may be implemented in pacemaker 14 for charging a pacing capacitor to a predetermined pacing pulse amplitude under the control of control circuit 206 and delivering a pacing pulse.

Memory 210 may include computer-readable instructions that, when executed by control circuit 206, cause control circuit 206 to perform various functions attributed throughout this disclosure to pacemaker 14. The computer-readable instructions may be encoded within memory 210. Memory 210 may include any non-transitory, computer-readable storage media including any volatile, non-volatile, magnetic, optical, or electrical media, such as a random access memory (RAM), read-only memory (ROM), non-volatile RAM (NVRAM), electrically-erasable programmable ROM (EEPROM), flash memory, or other digital media with the sole exception being a transitory propagating signal. Memory 210 may store timing intervals and other data used by control circuit 206 to control the delivery of pacing pulses by pulse generator 202, e.g., by sensing an atrial event signal by atrial event detector circuit 240 from the motion signal received from motion sensor 212 and setting a pacing escape interval timer included in pace timing circuit 242, according to the techniques disclosed herein.

Power source 214 provides power to each of the other circuits and components of pacemaker 14 as required. Power source 214 may include one or more energy storage devices, such as one or more rechargeable or non-rechargeable batteries. The connections between power source 214 and other pacemaker circuits and components are not shown in FIG. 3 for the sake of clarity but are to be understood from the general block diagram of FIG. 3. For example, power source 214 may provide power as needed to charging and switching circuitry included in pulse generator 202, amplifiers, ADC 226 and other components of sensing circuit 204, telemetry circuit 208, memory 210, and an accelerometer, filters, amplifiers, ADC, rectifier and other components as needed of motion sensor 212.

Telemetry circuit 208 includes a transceiver 209 and antenna 211 for transferring and receiving data via a radio frequency (RF) communication link. Telemetry circuit 208 may be capable of bi-directional communication with external device 50 (FIG. 1) as described above. Motion sensor signals and cardiac electrical signals, and/or data derived therefrom may be transmitted by telemetry circuit 208 to external device 50. Programmable control parameters and algorithms for performing cardiac event signal sensing and pacing therapy control may be received by telemetry circuit 208 and stored in memory 210 for access by control circuit 206.

The functions attributed to pacemaker 14 herein may be embodied as one or more processors, controllers, hardware, firmware, software, or any combination thereof. Depiction of different features as specific circuitry is intended to highlight different functional aspects and does not necessarily imply that such functions must be realized by separate hardware, firmware or software components or by any particular circuit architecture. Rather, functionality associated with one or more circuits described herein may be performed by separate hardware, firmware or software components, or integrated within common hardware, firmware or software components. For example, atrial event signal sensing from the motion signal and ventricular pacing control operations performed by pacemaker 14 may be implemented in control circuit 206 executing instructions stored in memory 210 and relying on input from sensing circuit 204 and motion sensor 212. Providing software, hardware, and/or firmware to accomplish the described functionality in the context of any modern pacemaker, given the disclosure herein, is within the abilities of one of skill in the art.

FIG. 4 is an example of a motion sensor signal 250 that may be sensed by pacemaker motion sensor 212 over a cardiac cycle. Vertical dashed lines 252 and 262 denote the timing of two consecutive ventricular events (an intrinsic ventricular depolarization or a ventricular pacing pulse), marking the respective beginning and end of the ventricular cycle 251. As used herein, a cardiac cycle refers to one cycle of a ventricular systolic phase and a ventricular diastolic phase and may also be referred to as a “ventricular cycle” because it starts and ends with ventricular events 252 and 262.

The motion signal 250 includes cardiac event signals labeled as an A1 event 254, an A2 event 256, an A3 event 258 and an A4 event 260. The A1 event 254 is an acceleration signal (in this example when motion sensor 212 is implemented as an accelerometer) that occurs during ventricular contraction and marks the approximate onset of ventricular mechanical systole. The A2 event 256 is an acceleration signal that may occur with closure of the aortic and pulmonic valves, marking the approximate offset or end of ventricular mechanical systole. The A2 event may also mark the beginning of the isovolumic relaxation phase of the ventricles that occurs with aortic and pulmonic valve closure.

The A3 event 258 is an acceleration signal that occurs during passive ventricular filling and marks ventricular mechanical diastole. The A3 event can also be referred to as the “passive ventricular filling event.” The A4 event 260 is an acceleration signal that occurs during ventricular diastole during a normal cardiac cycle and corresponds to atrial contraction and active ventricular filling. The A4 event 260 marks atrial mechanical systole. The A4 event 260 is also referred to herein as the “atrial systolic event signal” or merely the “atrial event signal” that is sensed from motion signal 250 by pacemaker control circuit 206. When atrial event detector circuit 240 senses A4 event 260, pace timing circuit 242 can be triggered to start an AV pacing interval when control circuit 206 is operating in AVS pacing mode. During a non-atrial tracking ventricular pacing mode, A4 events may be sensed by control circuit 206. The A4 events, however, may not be sensed during all ventricular cycles because they may occur anywhere during the ventricular cycle when the ventricles are being paced asynchronously with the atrial rhythm.

Techniques described below in conjunction with FIGS. 6-14 may be performed by processing circuitry of the medical device system 10 of FIG. 1 for establishing AVS pacing control parameters used for sensing A4 events attendant to atrial contraction from a motion signal. The motion signal received from motion sensor 212 during an asynchronous ventricular pacing mode may be characterized by processing circuitry of pacemaker 14 and/or external device 50 by determining features of the motion signal during cardiac cycles that are accepted based on P-wave timing. Determined motion signal features are used in establishing AVS pacing control parameters, e.g., atrial event sensing parameters. Using the techniques disclosed herein, cardiac signal analyzer 51 may provide P-wave timing markers or signals used by processing circuitry of the medical device system 10 for rejecting ventricular cycles during which atrial systole, as evidenced by P-wave identification by cardiac signal analyzer 51, is not confirmed to occur during ventricular diastole. Features of the motion signal sensed during rejected cardiac cycles are not determined or, if already determined prior to rejecting a corresponding cardiac cycle, not used by the processing circuitry in establishing AVS pacing control parameters.

FIG. 5 depicts example of motion signals 400 and 410 sensed by motion sensor 212 over two different cardiac cycles. A ventricular pacing pulse is delivered at time 0.0 seconds for both cardiac cycles during an asynchronous ventricular pacing mode. The top motion signal 400 is received over one cardiac cycle, and the bottom sensor signal 410 is received over a different cardiac cycle. The two signals 400 and 410 are aligned in time at 0.0 seconds, the time of the ventricular pacing pulse delivery. While motion signals 400 and 410 and motion signal 250 of FIG. 4 are shown as raw accelerometer signals, it is recognized that control circuit 206 may receive a digitized filtered, amplified and rectified signal from motion sensor 212 for processing and analysis as described herein in conjunction with accompanying drawings.

The A1 events 402 and 412 of the respective motion signals 400 and 410, which occur during ventricular contraction, are observed to be well-aligned in time following the ventricular pacing pulse at time 0.0 seconds. Similarly, the A2 events 404 and 414 (which may mark the end of ventricular systole and the isovolumic ventricular relaxation phase) and the A3 events 406 and 416 (occurring during the passive ventricular filling phase of ventricular diastole) are well-aligned in time. Since the A1, A2 and A3 event signals are each associated with ventricular events, each of these event signals are expected to occur at relatively consistent intervals in the motion signal following a ventricular electrical event, the ventricular pacing pulse in this example, and relative to each other. The time relationship of the A1, A2 and A3 events may be different following a ventricular pacing pulse compared to following a sensed intrinsic R-wave and/or at different ventricular rates. However, during a stable paced or intrinsic ventricular rhythm, the relative timing of ventricular A1, A2 and A3 events to each other and the immediately preceding ventricular electrical event is expected to be consistent from beat-to-beat.

The A4 events 408 and 418 of the first and second motion sensor signals 400 and 410 respectively are not aligned in time. The A4 event occurs during atrial systole and is associated with the atrial kick or active ventricular filling phase of ventricular diastole. The time interval of the A4 event following the immediately preceding ventricular electrical event (sensed R-wave or ventricular pacing pulse) and the preceding A1 through A3 events may vary between cardiac cycles during an asynchronous ventricular pacing mode (and in some instances during AVS pacing, e.g., when the atrial rate is changing).

The consistency of the timing of the A1 through A3 events relative to each other and the immediately preceding ventricular electrical event may be used for determining PVABP 436 and increasing confidence in reliably sensing A4 event 408 and 418. Atrial event detector circuit 240 does not sense A4 events during PVABP 436, which may extend from the ventricular electrical event (at time 0.0) through an estimated onset of ventricular diastole so that PVABP 436 generally encompasses both the A1 and A2 event signals.

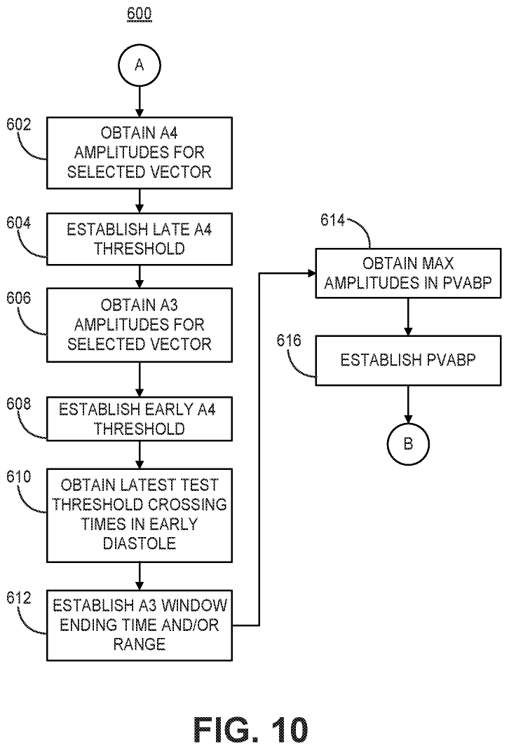

An A3 window 424, also referred to herein as the “passive ventricular filling window,” may be set by atrial event detector circuit 240 having a starting time 420 corresponding to the end of PVABP 436 and an ending time 422. The ending time 422 may be established using techniques described below, e.g., in conjunction with FIG. 10. The ending time 422 may also be considered a starting time of an A4 sensing window 450, though A4 events may be sensed during the A3 window in some instances. As such, the A3 window may also be referred to as a “sensing window,” however a different A4 sensing threshold amplitude may be applied to the motion signal by atrial event detector circuit 240 during the A3 window than after the A3 window ending time.

A4 events 408 and 418 may be detected based on a multi-level A4 sensing threshold 444. As seen by the lower motion sensor signal 410, the A4 event 418 may occur earlier after the A3 window 424 due to changes in atrial rate relative to the paced ventricular rate. In some instances, as the atrial rate increases even during an AVS pacing mode, the A4 event 418 may occur within the A3 window 424. When this occurs, the A3 event 416 and the A4 event 418 may fuse as passive and active ventricular filling occur together during ventricular diastole. The fused A3/A4 event may have a high amplitude, even greater than the amplitude of either the A3 event 416 or the A4 event 418 when they occur separately. As such, in some examples a first, high A4 sensing threshold amplitude 446 may be established for sensing an early A4 event that is fused with the A3 event during the A3 window 424. A second, low A4 sensing threshold amplitude 448 (also referred to herein as the “late A4 sensing threshold amplitude” because it is applied relatively later in the cardiac cycle) may be established for sensing relatively late A4 events, after the ending time 422 of the A3 window 424, during an A4 window 450.

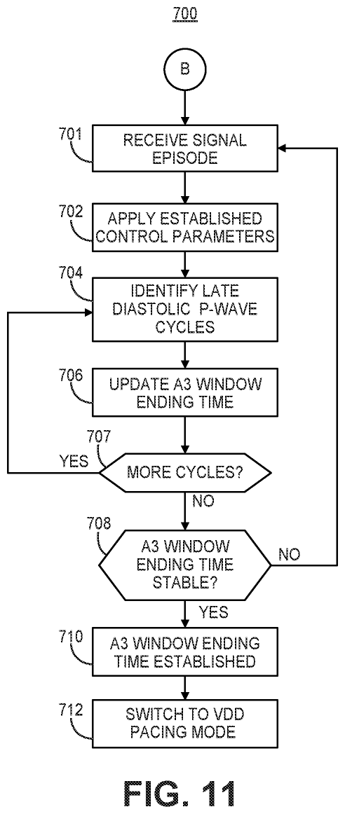

The A4 window 450 extends from the ending time of the A3 window 424 until the A4 event is sensed or until the next ventricular electrical event, sensed or paced (not shown in FIG. 5), whichever occurs first. The earliest crossing of the A4 sensing threshold 444 by the motion signal after the starting time 420 of the A3 window (or after the expiration of PVABP 436) may be sensed as the atrial event signal. Techniques for establishing an early A4 sensing threshold amplitude 446 applied to the motion signal during the A3 window 424 and a late A4 sensing threshold amplitude 448 applied to the motion signal after the ending time 422 of the A3 window 424, during the A4 window 450, are described below, e.g., in conjunction with FIGS. 10-11.

In FIG. 5, a late diastolic (LD) time period 452 is shown in this example as a time interval beginning after the ending time 422 of the A3 window 424 and extending until the next ventricular event (not shown in FIG. 5). The LD time period 452 may be defined for use by processing circuitry of pacemaker 14 and/or external device 52 in accepting and rejecting cardiac cycles used for establishing AVS pacing control parameters according to the techniques disclosed herein. The LD time period 452 may correspond to the A4 window 450 in some examples. Processing circuitry of the medical device system 10 of FIG. 1 receives P-wave timing markers from cardiac signal analyzer 51 that mark the relative timing of P-waves identified by cardiac signal analyzer 51 following a ventricular event that starts a cardiac cycle.

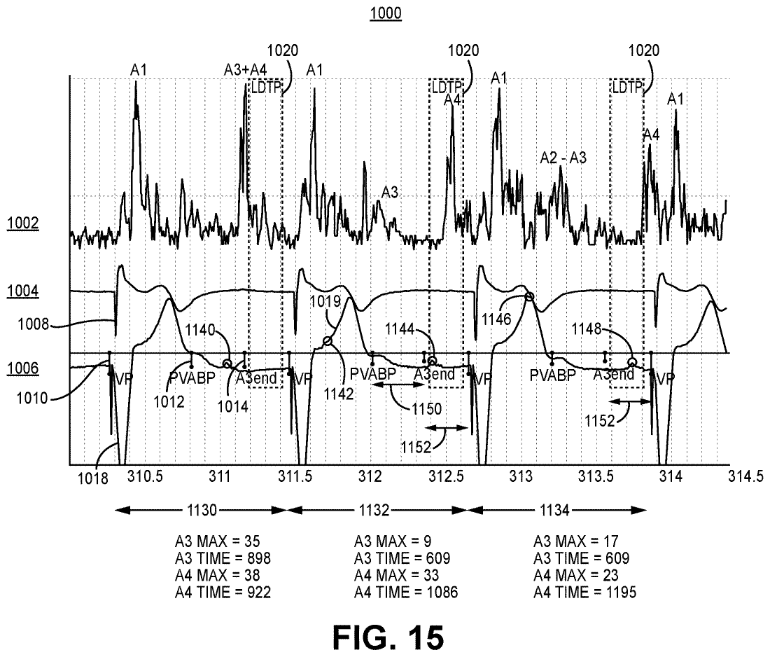

An LD threshold time that defines the starting time of the LD time period 452 following a ventricular event may be between 800 ms and 1200 ms or between 900 and 1000 ms, as examples, and may be based on the ventricular rate. A relatively longer LD threshold time may be set when the pacing rate is relatively slow, e.g., 50 bpm or less. The LD threshold time may be set relatively shorter following a ventricular event when the pacing rate is relatively faster, e.g., 60 bpm or faster. The LD time period 452 may end when the A4 window 450 ends upon the next ventricular pacing pulse (or sensed R-wave). In other examples, as described below in conjunction with FIG. 15, the LD time period 452 may have an ending time that is earlier than the end of the A4 window 450. The A4 event in the motion signal may occur 50 to 150 ms after a P-wave due to the electro-mechanical delay between atrial myocardial depolarization and atrial contraction. As such, in order to identify cardiac cycles that have an A4 event during the A4 window 450 and before the onset of the next cardiac cycle, e.g., before the next ventricular pacing pulse, the LD time period 452 may have an ending time that is 50 to 200 ms before the end of the A4 window 450.

When the P-wave timing marker indicates that the time of an identified P-wave during the LD time period 452 of a cardiac cycle, the cardiac cycle can be accepted for use in establishing AVS pacing control parameters. When a P-wave timing marker is absent from the LD time period 452 (no P-wave is identified during the LD time period 452 of the current cardiac cycle or is identified but with a low level of confidence), the cardiac cycle may be rejected for use in establishing AVS pacing control parameters. Features of the motion signal sensed during rejected cardiac cycles may not be determined or may not be used in establishing the AVS pacing control parameters according to the techniques described below.

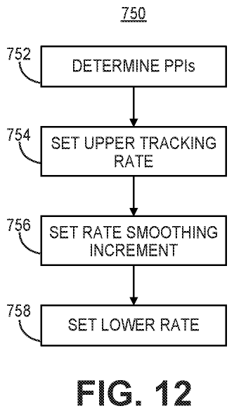

P-wave timing markers output by cardiac signal analyzer 51 during accepted and rejected cardiac cycles may, however, be used in establishing some AVS pacing control parameters. For example, as described below in conjunction with FIG. 12, some AVS pacing control parameters may be established by processing circuitry of medical device system 10 based on PP intervals determined from P-wave timing markers output by cardiac signal analyzer 51, regardless of the relative timing of the P-wave timing markers during a respective cardiac cycle. The P-wave timing markers may indicate the patient's true atrial rate, which may be used in setting a rate response interval increment or other pacing related control parameters as described below.

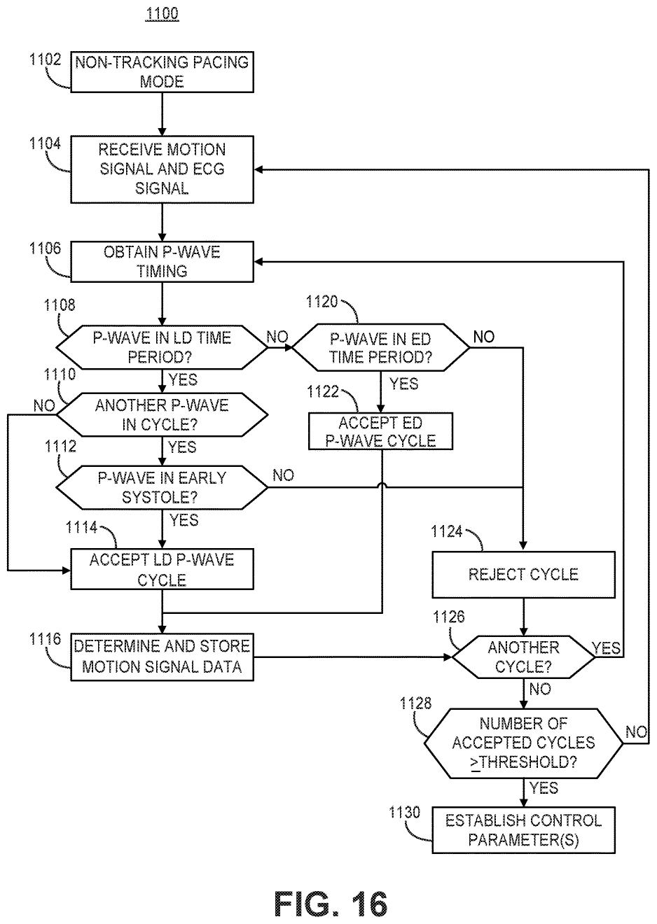

The LD time period 452 may start after expiration of PVABP 436 and may start at or later than the ending time 422 of A3 window 424. In the example shown, LD time period 452 starts at 1.0 second after the ventricular pacing pulse. In other examples, the LD time period 452 may start 800 ms to 1100 ms after ventricular event, sensed or paced. When a P-wave timing marker output by cardiac signal analyzer 51 indicates a P-wave occurs after a threshold time interval defining the start of the LD time period 452, features of the motion signal determined from that cardiac cycle can be determined and used for establishing one or more AVS pacing control parameters. Depending on the atrial rate, a P-wave timing marker may be output by cardiac signal analyzer 51 before LD time period 452 and during the LD time period 452 in the same cardiac cycle. Two P-waves could occur during one asynchronous ventricular pacing cycle when the ventricular pacing rate is relatively slow compared to the atrial rate, for example. The motion signal data from a cardiac cycle that includes a P-wave before and during the LD time period 452 may or may not be rejected by the processing circuitry for use in determining AVS pacing control parameters. For example, as described below in conjunction with FIGS. 15 and 16, processing circuitry of the medical device system may accept a cardiac cycle when a P-wave timing marker is during an early systolic portion of the cardiac cycle and another P-wave timing marker is during the LD time period 452 of the same cardiac cycle. A cardiac cycle with a P-wave timing marker during a late systolic portion and/or early diastolic portion of the cardiac cycle may be rejected in some examples.

FIG. 6 is a flow chart 300 of a method for establishing AVS pacing control parameters according to some examples. The process of flow chart 300 may be performed by medical device system 10 of FIG. 1 upon implantation of pacemaker 14 and may be performed at other post-implant times for updating or resetting one or more AVS pacing control parameters. Processing circuitry of medical device system 10 may perform the method of flow chart 300. For the sake of ease, the process of flow chart 300 and other flow charts presented herein are described as generally being performed by external device processor 52, e.g., in conjunction with receiving a motion signal and/or data derived from the motion signal from pacemaker 14. In other examples, control circuit 206 may perform processing and analysis for establishing AVS pacing control parameters using output received from cardiac signal analyzer 51, which may be implemented in control circuit 206 or external device processor 52 in which case the cardiac signal analyzer output may be transmitted to pacemaker 14.

When the processing and analysis of one or more ECG and/or EGM signal(s) and the motion signal are performed by external device processor 52 for establishing AVS control parameters, pacemaker pulse generator 202, sensing circuit 204, motion sensor 212, memory 210 and telemetry circuit 208 may be involved in the process of flow chart 300 for delivering ventricular pacing pulses, sensing the motion signal, storing one or more motion signal episodes and/or data determined from the motion signal episode(s) in memory 210, and transmitting motion signal data via telemetry circuit 208 to external device 50 and optionally transmitting an EGM signal as needed by external device processor 52 for establishing AVS pacing control parameters. However, it is to be understood, that processing circuitry of pacemaker 14 may perform at least a portion or all of the signal processing and analysis described in conjunction with flow chart 300 in some examples. Pacemaker 14 and external device 50 may be configured to perform the automatic setup procedure individually or cooperatively for establishing AVS pacing control parameters according to the techniques disclosed herein.

At block 302, control circuit 206 of pacemaker 14 sets the pacing mode to a non-atrial tracking ventricular pacing mode (e.g., VDI), so that ventricular pacing pulses are being delivered asynchronously to atrial events. The pacing rate may be set to a nominal rate, e.g., between 30 and 80 pulses per minute or about 40 to 60 pulses per minute.

Ventricular electrical events during the VDI pacing mode will generally be delivered ventricular pacing pulses in a patient with AV block but may include intrinsic R-waves in some instances, particularly in a patient with intermittent AV conduction. The ventricular rate may be intentionally set to be different than the atrial rate if the atrial rate is known, e.g., based on user input or as determined based on intervals between consecutive P-wave timing outputs from cardiac signal analyzer 51. External device 50 may transmit a set-up command that to pacemaker 14 includes a command to switch to an asynchronous ventricular pacing mode and a ventricular pacing lower rate to set the lower rate to be different than the atrial rate, e.g., slower (or faster) than the atrial rate.

In a patient having AV block, atrial systolic events generally occur asynchronously with ventricular electrical events during the non-atrial tracking, asynchronous ventricular pacing mode. As such, atrial event signals may course through the cardiac cycle at varying times during the VDI pacing mode. In order to avoid having all P-waves occurring coincidentally with and superimposed on the R-waves of a cardiac electrical signal received by cardiac signal analyzer 51, the asynchronous paced ventricular rate may be set to be different than the atrial rate. Alternatively, the ventricular pacing lower rate may be varied to promote asynchrony of the atrial P-waves and the ventricular pacing pulses to encourage at least some atrial P-waves during the ventricular diastolic phase. For example, the ventricular pacing lower rate may be increased and/or decreased between a maximum lower rate and a minimum lower rate. The lower rate may be increased and/or decreased according to predetermined step increments or decrements, e.g., 50 to 200 ms, to avoid abrupt changes in ventricular rate. One or more ventricular pacing pulses may be delivered at each rate step.

At block 304, processing circuit 52 of external device 50 may receive a motion signal episode and ECG signal episode (or EGM signal episode). The motion signal episode can include an axis signal corresponding to each axis of the multi-axis motion sensor. In the illustrative examples presented herein, the motion sensor is a three-dimensional accelerometer having three axes which may be referred to as axis 1, axis 2 and axis 3. Each of the three axis signals may be transmitted to processor 52 for analysis as individual single axis signals, combinations of two axis signals and/or a combination of all three axis signals. As described below, processor 52 may select a motion signal sensing vector as one axis, a combination of two axes, or a combination of all three axes of the motion sensor. The motion signal sensing vector may be established as an AVS pacing control parameter based on the signal processing and analysis described below. In other examples, a default or previously selected motion signal sensing vector signal may be sensed and transmitted from pacemaker 14 as the motion signal episode. Processing circuit 52 may be configured to process and analyze the motion signal episode and cardiac electrical signal(s) for establishing other atrial sensing control parameters, such as the A3 window ending time and/or the early, high A4 sensing threshold amplitude and the late, low A4 sensing threshold amplitude.

The motion signal (which may refer collectively to each axis signal of the motion sensor) may be received by external device 50 from pacemaker 14 contemporaneously with at least one ECG signal sensed and recorded via surface electrodes 57 over a specified time interval or number of cardiac cycles (e.g., specified number of delivered ventricular pacing pulses). For example, processing circuit 52 may be configured to start receiving a motion signal transmitted from pacemaker 14 in real time, with ventricular pacing pulse markers, to enable processing circuit 52 to sample an ECG signal segment for the same cardiac cycles, e.g., starting from a ventricular pacing pulse of a first cardiac cycle. One or more ECG signals may be sampled from one or more pairs of surface electrodes 57 as needed for input to cardiac signal analyzer 51.

The ECG signal(s) and the motion signal may be time aligned beginning from a starting ventricular pacing pulse. A predetermined number of cardiac cycles or a predetermined time duration of the signals may be acquired as a signal episode. For example, an episode of the motion signal and at least one ECG signal may be received over a few seconds to several minutes or longer, e.g., over about 5 seconds to two minutes or over about 10 seconds to 60 seconds as examples. The signal episode may include ventricular pacing pulse markers transmitted with the motion signal data from pacemaker 14. The pacing pulse markers indicate the relative timing, e.g., based on sample number or specified in milliseconds (ms), of ventricular pacing pulses delivered during the signal episode. The motion signal and ECG signal(s) may be stored in external device memory 53 for post-processing and analysis. In other examples, the operations of accepting or rejecting cardiac cycles and accumulating motion signal features from accepted cardiac cycles from the signal episode may be performed beat by beat in real time as the signals are received (with any necessary processing time delays).

For the sake of ease, flow chart 300 is described assuming all cardiac cycles of the signal episode are paced ventricular cycles, each starting with an asynchronous ventricular pacing pulse. The cardiac cycles of the signal episode may all be paced ventricular cycles because the patient may have total AV conduction block and/or the ventricular pacing rate may be faster than the intrinsic atrial rate. However, it is to be understood that in some cases, one or more cardiac cycles during the signal episode may be sensed ventricular cycles, starting with an intrinsic R-wave instead of a ventricular pacing pulse. In this case, the time of a P-wave identified by cardiac signal analyzer 51 from an R-wave may be determined by processor 52 for determining if the P-wave occurs after an LD threshold time that marks the start of the LD time period. The R-wave may be identified by cardiac signal analyzer 51 or processor 52 or be identified based on a ventricular sensed event marker signal received with the motion signal episode from pacemaker 14. Processor 52 may determine the P-wave timing during a sensed ventricular cycle for use in accepting or rejecting the sensed ventricular cycle in accumulating motion signal data, as generally described below. In some examples, all sensed ventricular cycles may be rejected cycles.

At block 306, processor 52 may input one or more received ECG signal(s) to cardiac signal analyzer 51 to obtain P-wave timing marker signals for at least some of the cardiac cycles in the motion signal episode received from pacemaker 14. It is to be understood that depending on the relative ventricular and atrial rates, there may be cardiac cycles with no P-wave during the ventricular cycle, one P-wave during the ventricular cycle, or multiple P-waves during the ventricular cycle. Assuming the atrial rate is equal to or faster than the asynchronous ventricular pacing rate, at least one P-wave may be expected to be identified by cardiac signal analyzer 51 during each cardiac cycle and in some instances more than one. The P-wave may occur at varying time points in the cardiac cycle from one cycle to the next during asynchronous ventricular pacing.

Starting with the first cardiac cycle of the signal episode, processing circuit 52 may receive the output of cardiac signal analyzer 51 and classify the corresponding cardiac cycle as accepted or rejected based on the time of the identified P-wave during the cardiac cycle. The output of cardiac signal analyzer 51 may be a sample number that may be used to annotate an ECG and/or motion signal to indicate the timing of a P-wave attendant to atrial depolarization during a cardiac cycle with a high level of confidence. Processor 52 may compare the sample number of the P-wave identified by cardiac signal analyzer 51 to the sample number of the most recent ventricular pacing pulse. When the difference between the two sample numbers (or corresponding time interval) indicate that the P-wave is identified from the ECG signal after the LD threshold time at block 308, processor 52 classifies the associated cardiac cycle as accepted at block 310. If the P-wave is identified earlier in the cardiac cycle than the LD threshold time, processor 52 may classify the cardiac cycle as rejected at block 314, as long as no other P-wave is identified during the LD time period in the same cardiac cycle. In this way, the processor 52 identifies diastolic P-wave cycles from the cardiac signal episode as cardiac cycles with a P-wave timing marker during the ventricular diastolic phase of the cardiac cycle, e.g., during the LD time period that starts from the LD threshold time after a ventricular pacing pulse (or identified R-wave) and ends with or before the end of the A4 window.

In some examples, the output of cardiac signal analyzer 51 may include a time in milliseconds or may be converted to a time in milliseconds by processor 52 indicating the time interval from the most recent preceding ventricular pacing pulse to the detected P-wave. The time in milliseconds may be compared to the LD threshold time for rejecting cardiac cycles in which the P-wave occurs before the LD time period and accepting cardiac cycles in which the P-wave is identified in the ECG signal(s) in the LD time period after the most recent ventricular pacing pulse.

In other instances, processor 52 may determine an unknown P-wave timing when a P-wave cannot be identified during the cardiac cycle at all or is identified during the LD time period but with less than a threshold level of confidence. For instance, cardiac signal analyzer 51 may output a P-wave timing marker with a corresponding level of confidence (e.g., in a range of 0 to 100%). The level of confidence in identifying a P-wave by cardiac signal analyzer 51 may be relatively low in some instances. When the P-wave timing is output by cardiac signal analyzer 51 with a level of confidence less than a threshold level, e.g., less than 90%, less than 80%, less than 70%, less than 60%, less than 50% or less than another threshold percentage, processor 52 may determine that the P-wave timing is unknown and may reject the cardiac cycle.

When the current cardiac cycle is rejected at block 314, processor 52 may determine if there is another cycle in the signal episode. If so, processor 52 may return to block 306 to fetch the next P-wave timing output of cardiac signal analyzer 51 corresponding to the next cardiac cycle in the episode.

When the P-wave timing marker is later than the LD threshold time as determined at block 308 (and before the end of the LD time period) so that the cardiac cycle is accepted at block 310, processor 52 may determine motion signal features from the accepted cardiac cycle at block 312. Processor 52 may determine features of the motion signal episode for each of the accepted cardiac cycles. The motion signal features may be determined from the motion signal sensed during accepted cardiac cycles during various portions of the cardiac cycle, e.g., during the PVABP, during an early diastolic (ED) time period after the PVABP, and/or during an LD time period. The motion signal features may be determined for one or more motion signal sensing vectors, each of which may be a single axis signal or a combination of two or all three axis signals. The motion signal features that are determined may characterize the amplitude of the motion signal during ventricular systole and/or during the passive and/or active filling phases of ventricular diastole of each accepted cardiac cycle.

For example, at block 312, external device processor 52 may determine the motion signal maximum amplitude in one or more of the accepted cardiac cycles. In some examples, external device processor 52 may additionally or alternatively determine a time of the latest negative-going crossing of a test threshold amplitude by the motion signal as further described below. The data acquired at block 312 may be acquired during one or more accepted cardiac cycles of the signal episode for each available motion sensor signal vector or one or more predetermined motion sensor signal vectors selected for evaluation, which may include sensing vectors defined by any combination of one or more axis signals of motion sensor 212.

In other examples, the motion signal features for each cardiac cycle in the signal episode may be determined by control circuit 206 of pacemaker 14 and transmitted to external device 50 (e.g., at block 304) for the signal episode. The motion signal features determined for each cardiac cycle may be transmitted in real time as they are determined or buffered in pacemaker memory 210 in association with each ventricular pacing pulse or cardiac cycle number of the motion signal episode. External device processor 52 may select the motion signal feature data transmitted from pacemaker 14 that corresponds to one or more accepted cardiac cycles and discard the motion signal feature data corresponding to rejected cardiac cycles for determining AVS pacing control parameters.

After determining and storing the motion signal feature data for the current cardiac cycle at block 312, processor 52 may determine if there is another cardiac cycle in the signal episode at block 316. If so, processor 52 may return to block 306 and repeat the process of either accepting or rejecting the next cardiac cycle and obtaining and storing the motion signal feature data if the next cycle is accepted until all cardiac cycles of the signal episode have been evaluated. In other examples, processor 52 may evaluate each cardiac cycle until a desired number of accepted cardiac cycles has been reached and then advance to block 320 to establish AVS pacing control parameters based on the accumulated motion signal feature data without evaluating any remaining cardiac cycles of the signal episode.

If the end of the episode is reached, processor 52 may determine at block 318 if a sufficient number of accepted cardiac cycles were identified in the signal episode. For example, processor 52 may compare the number of accepted cycles to a threshold number. If the number of accepted cycles is less than a threshold number, processor 52 may return to block 304 to obtain another signal episode and repeat the process of accepting or rejecting each cardiac cycle to accumulate additional motion signal feature data from accepted cardiac cycles. The threshold number of accepted cycles may be at least 5 cycles, at least 10 cycles, at least 20 cycles, at least 30 cycles, at least 50 cycles or at least 100 cycles as examples, with no limitation intended. The motion signal feature data determined from accepted cardiac cycles may be accumulated in external device memory 53 from a portion of a signal episode or from one or more signal episodes. Processor 52 may evaluate one or more signal episodes until a threshold number of accepted cycles is reached.

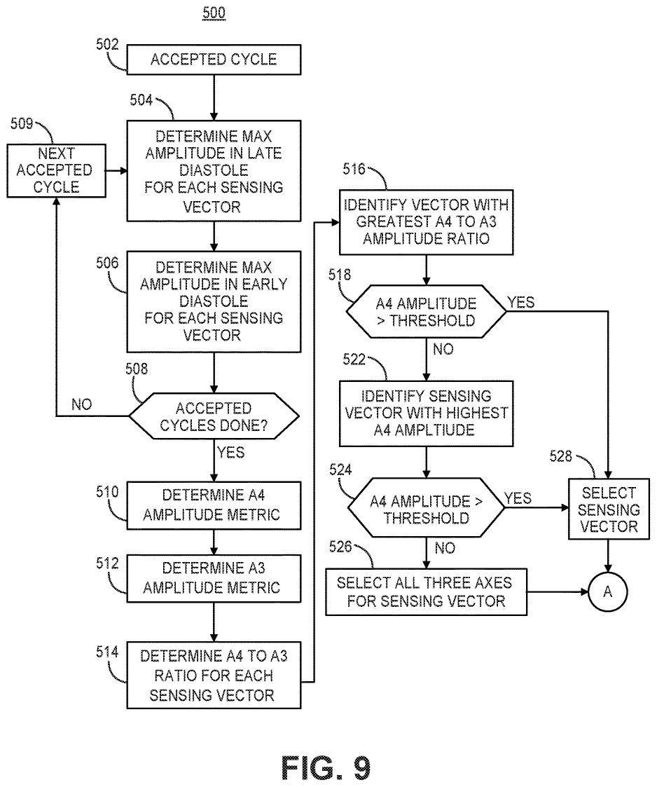

The motion signal features determined at block 312 may be stored in external device memory 53 in buffers allocated in memory 53 to enable processor 52 to analyze the determined features. Methods performed for determining and accumulating motion sensor signal data in memory 53 and establishing AVS pacing control parameters from the motion signal feature data are described below. For example, a log or histogram of motion signal maximum amplitudes may be populated in memory 53 for each available motion signal sensing vector. The maximum amplitudes may be analyzed for establishing a motion signal sensing vector as one or a combination of two or all three axis signals available from the motion sensor. Example methods for determining motion signal amplitude data that may be used in selecting a motion signal sensing vector are described below in conjunction with FIG. 9. Additionally or alternatively, motion signal maximum amplitude data may be determined and accumulated in memory 53 at block 312 for use in establishing an early A4 sensing threshold amplitude and a late A4 sensing threshold amplitude of the multi-level A4 sensing threshold applied to the motion signal by pacemaker atrial event detector circuit 240 for sensing atrial event signals. Example methods for determining motion signal amplitude data for establishing A4 sensing threshold amplitudes are described below, e.g., in conjunction with FIGS. 9-10.

In another example, the latest crossing time of a test threshold amplitude during the ED period of each accepted cardiac cycle may be stored in memory 53 at block 312 for use in establishing an ending time of the A3 window. Example techniques for establishing the A3 window ending time and fine tuning the A3 window ending time are described below in conjunction with FIGS. 10 and 11.

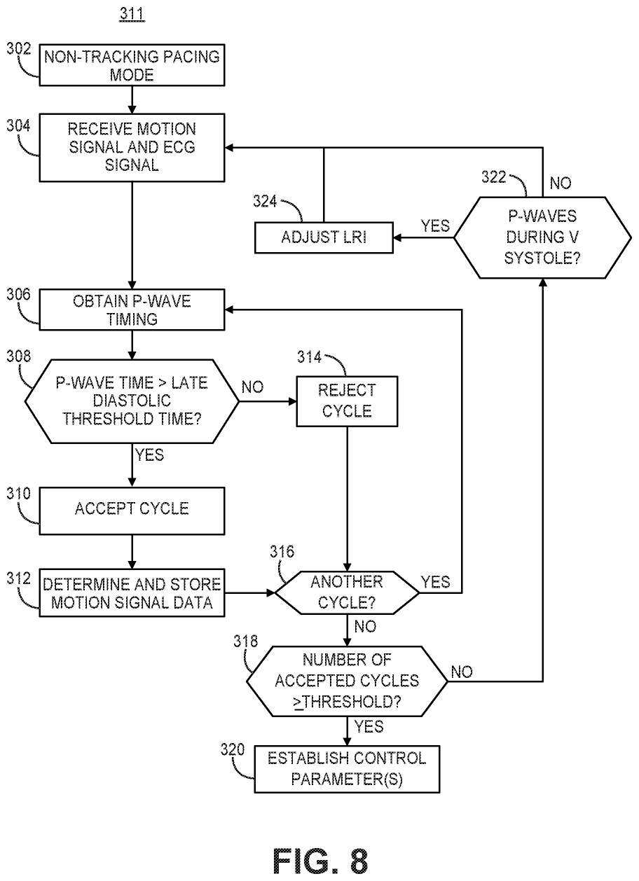

At block 320 of FIG. 6, processor 52 establishes control parameters used by pacemaker 14 during an AVS pacing mode. For example, processor 52 may analyze the motion sensor signal feature data accumulated for accepted cardiac cycles for establishing one or more AVS pacing control parameters. Based on the motion signal feature data, control circuit 206 may select an AVS pacing control parameter, which may include a motion signal sensing vector, the PVABP, an ending time of the A3 window, the early A4 sensing threshold amplitude applied to the motion signal during the A3 window, and/or the late A4 sensing threshold amplitude applied to the motion signal after the A3 window ending time. Various examples of techniques for determining AVS pacing control parameters from the motion signal data derived from the motion signal sensed during accepted cardiac cycles are described below, e.g., in conjunction with FIGS. 8-13.

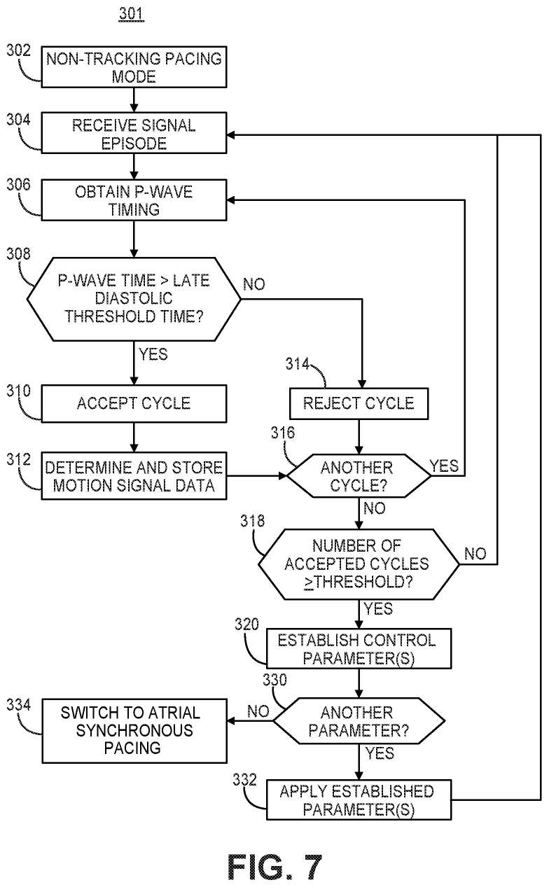

FIG. 7 is a flow chart 301 of a method that may be performed by processing circuitry of a medical device system 10 for establishing control parameters used by pacemaker 14 during an AVS pacing mode according to another example. Identically numbered blocks shown in FIG. 7 correspond to like-numbered blocks shown in FIG. 6 and generally described above. It is to be understood that the process of obtaining one or more signal episodes for accumulating motion signal data over a threshold number of accepted ventricular cycles may be performed multiple consecutive times for establishing multiple different control parameters used by pacemaker 14 during an AVS pacing mode. The setting of one control parameter, for example, may influence the selection of other control parameters that are established based on motion signal data determined from the accepted ventricular cycles. For example, as shown in FIG. 7, at least one control parameter may be established at block 320 based on at least one signal episode and threshold number of accepted cardiac cycles.

At block 330 processor 52 may determine if another control parameter remains to be established. If so, at block 332 processor 52 and/or pacemaker control circuit 206 may apply the control parameter(s) established at block 320, e.g., for sensing the motion signal, sensing A4 events, determining motion signal features and/or controlling ventricular pacing pulses during the VDI pacing mode. The established control parameter(s) may be applied by processor 52 and/or pacemaker control circuit 206 for acquiring a subsequent signal episode at block 304. One or more different control parameters may be established based on the analysis of the subsequent signal episode. During analysis of the motion signal during the subsequent episode, the same or different motion signal features may be determined and stored at block 312 for one or more accepted cardiac cycles, depending on the control parameters that are being established.

To illustrate, processor 52 may receive a first signal episode at block 304, e.g., a 10 to 30 second signal episode, that includes all available motion sensor axis signals. For each of the axis signals, processor 52 may determine a maximum amplitude of the axis signal during the LD time period of accepted ventricular cycles. Additionally, in some examples, processor 52 may determine a maximum amplitude of each axis signal during an ED time period, between the expiration of the PVABP and the start of the LD time period, of one or more accepted cardiac cycles. The ED time period may correspond to an A3 window in some examples. These maximum amplitudes may be used by processor 52 for establishing a motion signal sensing electrode vector for use by pacemaker 14 for sensing a motion signal and sensing atrial event signals from the motion signal. The established motion signal sensing vector may be one of the single axis signals or a combination, e.g., a summation, of two axis signals or all three axis signals in various examples.

After establishing the motion signal sensing vector at block 320, processor 52 may determine at block 330 that additional control parameters remain to be established. Additional control parameters may include the early A4 sensing threshold amplitude, the late A4 sensing threshold amplitude, the A3 window ending time, the PVABP or other parameters used in sensing atrial event signals from the selected motion signal sensing vector. Processor 52 may return to block 304 to obtain a subsequent signal episode using the selected motion signal sensing vector for accumulating motion signal data relevant for establishing control parameters for sensing A4 events from the selected motion signal. In some examples, different motion signal data or additional motion signal data may be determined from the subsequent signal episode. Processing time and burden may be conserved when the additional motion signal data is determined only from the selected motion signal instead of all available sensing vector signals.

In another example, processor 52 may establish the motion signal sensing vector and early and late A4 sensing threshold amplitudes at block 320 based on motion signal data obtained from one or more signal episodes. Processor 52 may return to block 304 to obtain another one or more signal episodes with the early and late A4 sensing threshold amplitudes in effect for acquiring motion signal data for adjusting an A3 window ending time. As further described below, the A3 window ending time may be set based on a latest, negative-going test threshold crossing of the motion signal during an ED time period of one or more accepted cardiac cycles. The test threshold crossing may be set based on the late A4 sensing threshold. The A3 window ending time established based on the latest crossing of the test threshold amplitude is therefore influenced by the established late A4 sensing threshold. As such, after establishing the early and late A4 sensing threshold amplitudes, processor 52 may receive at least one additional signal episode for establishing the A3 window ending time. Subsequent signal episodes acquired after one or more control parameters are established may be shorter than, equal to or longer than previous signal episodes.

In some examples, however, the motion signal data needed to establish all control parameters may be obtained and stored in external device memory 53 from one or more signal episodes without having to return to block 304 to obtain one or more additional signal episodes after establishing at least one control parameter. In still other examples, the one or more signal episodes received at block 304 may be processed and analyzed a first time for establishing one or more control parameters. The same one or more signal episodes including the threshold number of accepted ventricular cycles may be processed and analyzed a second time for establishing one or more additional control parameters using at least one previously established control parameter. For example, a signal episode may be analyzed once to establish the motion signal sensing vector from motion signal data determined from all available axis signals. The signal episode for the established motion signal sensing vector may be analyzed a second time to establish other atrial event sensing control parameters, e.g., an A4 sensing threshold amplitude, A3 window ending time, and/or the PVABP, based on motion signal data determined from the established sensing vector signal. Additional features of the motion signal may be determined from only the established sensing vector signal for establishing other control parameters. These additional motion signal features do not necessarily need to be determined from all available sensing vector signals (e.g., each motion sensor axis signal individually and in each possible combination of axis signals).

As described below, in addition to or alternatively to establishing atrial event sensing control parameters, processor 52 may establish ventricular pacing control parameters used by pacemaker control circuit 206 during an AVS pacing mode based on the motion signal data and/or an atrial rate that may be determined from the P-wave timing output of cardiac signal analyzer 51. For example, processor 52 may establish a ventricular pacing lower rate, a rate smoothing increment, the PVABP, and/or a maximum atrial tracking rate based on the atrial rate that may be determined from the P-wave timing output of cardiac signal analyzer 51.

When the process of establishing the AVS pacing control parameters according to flow chart 301 (or as described above in conjunction with flow chart 300 of FIG. 7) is complete, control circuit 206 may switch to an AVS pacing mode, e.g., a VDD pacing mode, at block 334. External device 50 may transmit a pacing mode switching command along with the established AVS pacing control parameters to pacemaker 14. The established AVS pacing control parameters may be in effect when atrial synchronous pacing begins.

FIG. 8 is a flow chart 311 of a method that may be performed by processing circuitry of medical device system 10 for accumulating motion signal feature data for establishing AVS pacing control parameters according to another example. Identically-numbered blocks in FIG. 8 correspond to like-numbered blocks shown in FIG. 6 as generally described above. In the example of FIG. 8, when less than the threshold number of accepted cycles has been reached at block 318, processor 52 may determine that one or more P-waves are occurring during ventricular systole in the signal episode at block 322. If the asynchronous ventricular pacing rate and the atrial rate are similar with atrial systole occurring approximately coincident with ventricular systole (or early diastole), processor 52 may reject a relatively high number of cardiac cycles during the signal episode. P-waves may be occurring, for example, during the QRS waveform, during the S-T segment, during the T-wave, or any time prior to the LD time period. Processor 52 may determine evidence of P-waves during ventricular systole (or early diastole) at block 322 in response to less than the threshold number of accepted cycles being reached at block 318. In another example, processor 52 may determine evidence of P-waves during ventricular systole at block 322 when greater than a threshold percentage of cardiac cycles are rejected from the current signal episode. For example, if greater than 50% or another selected percentage of cardiac cycles are rejected, processor 52 may determine evidence of P-waves during ventricular systole (or early diastole) in the current signal episode.

In still other examples, processor 52 may optionally determine at block 322 that P-waves are occurring during ventricular systole (or early diastole) based on P-wave timing output of cardiac signal analyzer 51 being during a ventricular systolic time interval. In some instances, cardiac signal analyzer 51 may output P-wave timing markers that occur within a threshold time interval of the ventricular pacing pulse, e.g., within the passive ventricular filling (A3) window ending time, within the PVABP, or within a specified systolic time interval threshold such as within 800 ms, 700 ms, 600 ms, 400 ms, 300 ms, 200 ms, 150 ms, or within 100 ms of the ventricular pacing pulse (or subsequent R-wave). When the P-wave timing output of cardiac signal analyzer 51 indicates a P-wave before an A3 window ending time, within the PVABP or another specified systolic time interval threshold from the ventricular pacing pulse (or R-wave), processor 52 may determine that the P-wave is occurring during ventricular systole (or early diastole). If no other P-wave occurs during the LD time period, the cardiac cycle can be determined to be a systolic P-wave cycle.

Processor 52 may count a rejected cardiac cycle as a possible systolic P-wave cycle when the P-wave is not identified during the ventricular cycle, when the only P-wave timing marker during the cardiac cycle is associated with a low level of confidence (e.g., less than 50% or other threshold percentage) and/or when an identified P-wave is within a systolic time interval threshold from the ventricular pacing pulse. If a threshold number of rejected cycles are identified as possible systolic P-wave cycles, processor 52 may determine evidence of P-waves during ventricular systole at block 322.

In still other examples, processor 52 may determine an atrial rate at block 322 based on PPIs. Processor 52 may determine time intervals between consecutively identified P-waves (also referred to as “PP intervals” or “PPIs”). The PPIs may be determined as the time interval between P-wave timing outputs of cardiac signal analyzer 51. Processor 52 may compare each PPI to the ventricular LRI and/or compare a mean, median or other representative value of the PPIs to the ventricular LRI used to deliver the asynchronous ventricular pacing pulses during the cardiac signal episode. If the PPIs are within a threshold range of the ventricular LRI, e.g., within 20 ms, 50 ms, 100 ms or other threshold range, processor 52 may determine evidence of P-waves during ventricular systole at block 322 due to a matching atrial and ventricular rate and fewer than the threshold number of accepted cycles being reached at block 318.

In response to determining evidence of P-waves during ventricular systole (or early diastole) and/or a matching ventricular and atrial rate, processor 52 may transmit a programming command to pacemaker 14 to adjust the pacing lower rate at block 324. To illustrate, if the ventricular lower rate is programmed to 50 beats per minute (bpm) and the atrial rate is close to 50 bpm, a relatively high percentage of the cardiac cycles during the signal episode may be cycles with the P-wave consistently during ventricular systole and/or early diastole. By changing the ventricular pacing lower rate, P-wave timing during the cardiac cycle can change or be more variable such that P-waves may shift to the late ventricular diastolic phase and/or course through the ventricular cycle from beat-to-beat resulting in at least some P-waves being in LD time period.

The ventricular pacing lower rate may be increased or decreased at block 324. The ventricular pacing lower rate may be increased or decreased to be at least 5, 10, 15 or 20 bpm greater than or less than the atrial rate corresponding to the PPIs. The ventricular pacing lower rate may be increased or decreased to be different than the atrial rate corresponding to the PPIs but within a maximum and/or minimum lower rate limit, e.g., within 30 to 80 bpm. The change in ventricular pacing lower rate from the current lower rate to the new lower rate may be made gradually over multiple ventricular cycles to avoid an abrupt change in ventricular rate in some examples.

In other examples, external device 52 may transmit a command at block 324 to pacemaker 14 to cause control circuit 206 to control pulse generator 202 to deliver at least one ventricular pacing pulse at a different LRI than the current LRI and then resume pacing at the current LRI. One or more ventricular pacing pulses delivered at a slightly shorter or slightly longer LRI, e.g., 50 to 500 ms shorter or longer, may shift the relative timing of the P-wave into the LD time period. After one or more ventricular pacing pulses are delivered at a different LRI, the currently programmed lower rate, e.g., 50 bpm, may be resumed with P-waves occurring more frequently during the LD time period even if the atrial rate is still similar to the ventricular pacing lower rate. In this way, the atrial systolic phase may be shifted relative to ventricular systole, into the ventricular diastolic phase and into the LD time period, to promote a higher number of accepted cardiac cycles during the next signal episode. One or more subsequent signal episodes may be received at block 304 after the rate adjustment for obtaining motion signal data from one or more accepted cardiac cycles and establishing AVS pacing control parameters at block 320.

FIG. 9 is a flow chart 500 of a method for selecting a motion signal sensing vector from the available motion sensor axis signals according to some examples. The motion signal sensing vector selection process of flow chart 500 can be performed for determining which vector signal (from one axis or a combination of axes) of a multi-axis motion sensor produces a motion signal from which atrial event signals are likely to be sensed most reliably, e.g., based on atrial event signal strength.

At block 502, processor 52 may identify an accepted cardiac cycle of a signal episode based on the P-wave timing marker from cardiac signal analyzer 51 as generally described above in conjunction with any of FIGS. 6-8. For each of the motion sensor vector signals to be analyzed, processor 52 may determine the motion signal peak amplitude during the LD time period of the accepted cardiac cycle at block 504. The LD time period may begin 800 to 1200 ms after a delivered ventricular pacing pulse (or a sensed R-wave) and extend until the next ventricular pacing pulse (or sensed R-wave) or until a specified ending time prior to the next scheduled ventricular pacing pulse. The LD time period starting and ending times may depend on the ventricular pacing rate. In an illustrative example, when the asynchronous ventricular pacing rate is 50 bpm, the LD time period starts 975 ms after the ventricular pacing pulse and extends to 1125 ms after the ventricular pacing pulse.

In some examples, each single axis signal, each possible combination of two axis signals (e.g., axis 1 plus axis 2, axis 1 plus axis 3 and axis 2 plus axis 3) and the combination of all three axis signals (axis 1 plus axis 2 plus axis 3) of a three-dimensional accelerometer are evaluated such that the maximum amplitude of the motion signal during the LD time period is determined for up to 7 possible motion signal sensing vectors. In other examples, a subset of the available sensing vectors may be evaluated, e.g., the three possible combinations of two-axis signals may be evaluated.

When a combination of two or all three axis signals are used to produce a vector signal, the motion signal sample points of the two or all three axis signals may be summed by processor 52 to produce a two-or three-axis vector signal. In other examples, the resultant vector signal may be determined using vector math. The maximum amplitude of a vector signal during an accepted cardiac cycle may be determined from the rectified vector signals.

Processor 52 may determine the maximum amplitude of the motion signal during the LD time period at block 504 for each motion signal sensing vector being evaluated. At block 506, processor 52 may determine the maximum amplitude of the motion signal during early diastole for each motion signal sensing vector being evaluated. Early diastole may be defined as extending from the expiration of the PVABP to the LD threshold time. Early diastole may be defined as being the A3 window, e.g., extending from the expiration of the PVABP to the A3 window ending time, which may be set nominally during the set up procedure to be 700 to 1000 ms after the ventricular pacing pulse. During an LD P-wave cycle, the maximum amplitude of the motion signal during early diastole may correspond to the amplitude of the true A3 event attendant to passive ventricular filling.

As such, each ventricular cycle that is accepted based on a truthed P-wave timing during the LD time period, a maximum motion signal amplitude during the LD time period is stored as a maximum amplitude of an A4 event (associated in time to the LD P-wave). A maximum motion signal amplitude during the ED time period is stored as an indication of the maximum amplitude of the A3 event because the A4 event is confirmed to be in late diastole based on the P-wave timing. The early diastole and late diastole motion signal maximum amplitudes may be determined and stored as A3 amplitudes and A4 amplitudes, respectively, for each motion signal sensing vector being evaluated.

If another accepted cardiac cycle is available in the signal episode (as determined at block 508), processor 52 may advance to the next accepted cycle at block 509 to obtain the motion signal amplitude data for the next accepted cycle. When the motion signal amplitude data has been determined from one or more accepted cardiac cycles of the signal episode for each sensing vector being evaluated (“yes” branch of block 508), processor 52 may determine an A4 amplitude metric at block 510 from all of the A4 maximum amplitudes stored at block 504. The A4 amplitude metric may be a median, mean, maximum, minimum, or other representative value of the A4 maximum amplitudes as examples. At block 512, processor 52 may determine an A3 amplitude metric from the A3 maximum amplitudes determined at block 506. The A3 amplitude metric may be a median, mean, maximum, minimum, or other representative value of the early diastole maximum amplitudes determined from LD P-wave cycles.

At block 514, processor 52 may determine the ratio of the A4 amplitude metric to the A3 amplitude metric for each of the sensing vectors being evaluated. At block 516, processor 52 may identify the sensing vector signal associated with the highest A4 to A3 amplitude ratio out of the single axis signals (if evaluated) and the two-axis signals being evaluated. At block 518, processor 52 may determine if a one axis or two axis motion signal sensing vector associated with the highest A4 to A3 amplitude ratio also has an A4 amplitude metric that is greater than a vector selection threshold amplitude. In other examples, processor 52 may determine if the sensing vector associated with the highest A4 to A3 amplitude ratio also has the highest A4 amplitude metric out of the available motion signal sensing vectors at block 518. If a motion signal sensing vector has the highest A4 to A3 amplitude ratio and an A4 amplitude metric that is greater than a vector selection amplitude threshold, processor 52 may select that sensing vector as the motion signal sensing vector at block 528.

If the A4 amplitude metric is not greater than the vector selection threshold amplitude at block 518 for the sensing vector having the highest A4 to A3 amplitude ratio, processor 52 may identify the single-axis or two-axis vector signal having the highest A4 amplitude metric at block 522. If the A4 amplitude metric is greater than a vector selection metric, processor 52 may select the sensing vector having the highest A4 amplitude metric at block 528. In this way, processor 52 may establish the motion signal sensing vector for use by pacemaker 14 for A4 event sensing to be the sensing vector having the highest A4 to A3 amplitude ratio identified at block 516 or the highest A4 amplitude metric identified at block 522 as long as the A4 amplitude metric is at least greater than a vector selection threshold amplitude. The vector selection threshold amplitude may be 0.8, 0.9 or 1.0 m/s² as examples.

In general, a single axis or a two axis motion signal sensing vector can be established for reliably sensing A4 events based on the actual signal strength of the A4 amplitude metric and/or the relative signal strength of the A4 amplitude metric compared to the A3 amplitude metric and/or other sensing vector A4 amplitude metrics. When a single or two axis motion signal sensing vector can be selected based on the criteria applied at blocks 516 and 518 or blocks 522 and 524, pacemaker power source 214 can be conserved by not having to power on more axes of the motion sensor than necessary for reliably sensing A4 events.

When none of the single-axis or two-axis vector signals meet the actual and/or relative signal strength requirements for vector selection applied at blocks 516 and 518 or blocks 522 and 524, processor 52 may select the combination of all three axis signals as the motion signal sensing vector at block 526. After establishing the motion signal sensing vector based on the motion signal amplitude data determined from one or more accepted LD P-wave cycles, processor 52 may advance to the flow chart 600 of FIG. 10 as indicated by connector “A”.

FIG. 10 is a flow chart 600 of a method that may be performed by processing circuitry of the medical device system of FIG. 1 for establishing control parameters used by pacemaker 14 during AVS pacing according to another example. In some instances, flow chart 600 is performed after establishing a motion signal sensing vector according to techniques described in conjunction with FIG. 9, as indicated by connector “A.” In other instances, the process of flow chart 600 may be performed after a motion signal sensing vector is selected by a user, e.g., by programming a desired sensing vector using external device 50. In still other examples, the process of flow chart 600 may be performed when a predetermined motion signal sensing vector is used as a default motion signal sensing vector. As such, in some instances the process of flow chart 600 may follow the process of flow chart 500 in FIG. 9 but does not necessarily follow the process of flow chart 500 because the motion signal sensing vector may be selected in other ways than the process of flow chart 500 or be previously established.

At block 602, processor 52 obtains the A4 amplitude(s) determined as the maximum motion signal amplitude during the LD time period of one or more accepted cardiac cycles of the selected sensing vector signal. As described above, the maximum amplitudes during the LD time period of LD P-wave cycles can correspond to the maximum amplitudes of true A4 events because of the evidence of the timing of truthed P-waves being in the LD time period. The A4 amplitude(s) may already be determined from one or more signal episodes and stored in memory 53, e.g., as described in the process of FIG. 9, for establishing the motion signal sensing vector. In other examples, a new signal episode may be received using the selected motion signal sensing vector for obtaining the A4 amplitudes at block 602.

At block 604, processor 52 may establish the late A4 sensing threshold amplitude that is applied during the A4 window by pacemaker control circuit 206 based on the A4 amplitudes. Processor 52 may set the late A4 sensing threshold amplitude to be less than the lowest A4 amplitude determined for the selected motion signal sensing vector. In this way, processor 52 establishes the late A4 sensing threshold amplitude at block 604 such that all of the A4 amplitudes stored for the selected sensing vector signal would be sensed as A4 events by atrial event detector circuit 240 of pacemaker 14. The late A4 sensing threshold amplitude is set to promote sensing by atrial event detector circuit 240 of all of the known A4 events (as confirmed based on the P-wave timing markers) during the accepted LD P-wave cardiac cycles.

The late A4 sensing threshold amplitude may be set to a fraction or percentage of the lowest A4 amplitude, e.g., to 70%, 80%, or 90% of the lowest A4 amplitude, or to a predetermined decrement, e.g., 0.1 to 0.3m/s², less than the lowest A4 amplitude. The late A4 sensing threshold amplitude may be set to be less than the minimum A4 amplitude stored for the selected sensing vector but not less than a minimum limit of the late A4 sensing threshold amplitude, e.g., not less than 0.6, 0.7 or 0.8m/s².

At block 606, processor 52 obtains the A3 amplitude(s) from the motion signal sensed using the selected sensing vector for one or more accepted cardiac cycles. The A3 amplitude(s) determined as the maximum motion signal amplitude during an ED period may correspond to the peak amplitudes of true A3 events when the accepted cycle is a LD P-wave cycle. At block 608, processor 52 may establish the early A4 sensing threshold amplitude applied to the motion signal during the A3 window by atrial event detector circuit 240 of pacemaker 14. The early A4 sensing threshold amplitude may be set to be greater than all of the A3 amplitudes determined at block 606. The early A4 sensing threshold amplitude may be set to be a percentage of or increment greater than the maximum A3 amplitude. In various examples, the early A4 sensing threshold may be set to be 110%, 115%, 120%, 130%, 150% or 200% of the maximum A3 amplitude. In other examples, the early A4 sensing threshold may be set to be 0.2, 0.3, 0.4, 0.5 or 1.0 m/s² or another increment greater than the maximum A3 amplitude obtained at block 606. Processor 52 may set the early A4 sensing threshold amplitude to be the greater one of a minimum limit of the early A4 sensing threshold or the specified percentage or increment greater than the A3 amplitude. The early A4 sensing threshold amplitude may be set to be greater than all A3 amplitudes to avoid oversensing an A3 event as a false A4 event.

At block 610, processor 52 may obtain the latest test threshold crossing time of the selected motion signal during the early diastole time period. In some examples, processor 52 may set the test threshold based on the late A4 sensing threshold amplitude. The test threshold amplitude may be set to a percentage, e.g., 75%, of the late A4 sensing threshold amplitude established at block 604. In other examples, the test threshold that may be set initially to a predetermined, fixed value, e.g., 0.8 m/s² to 1.2 m/s².

Processor 52 may obtain the latest test threshold crossing time by the motion signal during the ED time period at block 610 for at least one of the accepted LD P-wave cycles. The latest threshold crossing times may be determined for negative-going crossings of the test threshold. Processor 52 may determine the latest threshold crossing time(s) from one or more accepted cardiac cycles identified from one or more stored or newly received signal episodes. At block 612, processor 52 may establish the A3 window ending time and/or the allowable range of the A3 window ending times based on the latest test threshold crossing times. The latest test threshold crossing times may represent the shortest A3 window that reduces the likelihood of oversensing the A3 event as a false A4 event. If the A3 window is too short, the A3 event may occur or extend after the A3 window ending time, leading to possible oversensing of the A3 event during the early portion of the A4 window as a false A4 event.

As such, processor 52 may establish the A3 window ending time to be after the latest one of the negative-going test threshold crossing times at block 612. In other examples, processor 52 may determine the mean, median, maximum, minimum or other representative value of the latest test threshold crossing times during the ED time periods of multiple, accepted LD P-wave cycles. Processor 52 may set the A3 window ending time equal to the representative value of the latest test threshold crossing time or a predetermined increment (or percentage) longer than the representative value of the latest test threshold crossing time, e.g., 10 to 200 ms longer than the maximum latest test threshold crossing time.

In other examples, the A3 window ending time may be set based on the time of a maximum amplitude of the motion signal instead of a threshold crossing time. Processing 42 may determine the time of a maximum amplitude of the motion signal during the A3 window and/or the time of a maximum amplitude of the motion signal during the A4 window of accepted LD P-wave cycles. The A3 window ending time may be set based on the time of the maximum amplitude during the A3 window and/or the time of the maximum amplitude during the A4 window. For example, the A3 window ending time may be set half-way or at a different portion of the time interval between a mean, median, maximum or other representative value of the time of the A3 window maximum amplitude and a mean, median, minimum or other representative value of the time of the A4 window maximum amplitude.

Processor 52 may additionally or alternatively set an A3 window ending time range at block 612 that limits the minimum A3 window ending time and/or the maximum A3 window ending time that the A3 window ending time can be adjusted to by pacemaker control circuit 206 during (or in preparation for) atrial synchronous ventricular pacing. Pacemaker control circuit 206 may be configured to automatically adjust the A3 window ending time while operating in an AVS pacing mode. Examples of techniques for automatically adjusting the A3 window ending time are generally disclosed in U.S. patent application Ser. No. 17/159,596 (Sheldon, et al.) and U.S. patent application Ser. No. 17/159,635 (Sheldon, et al.).

Briefly, the late A4 sensing threshold amplitude may be adjusted by pacemaker control circuit 206 after a specified number of ventricular cycles during AVS pacing, e.g., after every 5 to 12 cycles, based on maximum peak amplitudes of A4 events sensed during the A4 window. Using the adjusted late A4 sensing threshold amplitude, pacemaker control circuit 206 may set an adjusted test threshold, e.g., to 70% to 80% of the late A4 sensing threshold amplitude. Control circuit 206 may apply the test threshold during the A3 window to obtain latest, negative-going test threshold crossing times during AVS pacing. After another predetermined number of ventricular cycles, which may be 5 to 30 cycles as examples, the A3 window ending time may be adjusted to or based on a median value of the latest test threshold crossing times. Pacemaker control circuit 206 may adjust the A3 window ending time to be applied during a next specified number of ventricular cycles based on the median latest test threshold crossing time. Pacemaker control circuit 206 may adjust the A3 window ending time up to a maximum A3 window ending time or down to a minimum A3 window ending time, e.g., within an A3 window ending time range.

At block 612, processor 52 may establish the A3 window ending time range based on a mean, median, minimum and/or maximum latest negative-going crossing time of the test threshold. For example, a minimum A3 window ending time limit may be set equal to or a predetermined time interval from (less than or greater than) the median latest test threshold crossing time. The maximum A3 window ending time limit may be set a predetermined time interval longer than the median latest test threshold crossing time. In an illustrative example, the A3 window ending time range may be established by processor 52 to be ±25 ms, ±50 ms, ±75 ms, or ±100 ms from the median latest test threshold crossing time determined from LD P-wave cycles. In other examples, the A3 window ending time range may be set to extend from a minimum A3 window ending time equal to the maximum latest negative-going crossing time of the motion signal during the ED time period to a maximum A3 window ending time that is 100 ms greater than the minimum A 3 window ending time.

In some examples, processor 52 may set the A3 window ending time range based on the latest test threshold crossing time (or the A3 window ending time established based on the latest test threshold crossing time) and the atrial rate. Processor 52 may determine the atrial rate based on PPIs determined from the P-wave timing markers output by cardiac signal analyzer 51. If the PPIs correspond to a relatively fast atrial rate, e.g., greater than 80, 85 or 90 bpm, the maximum A3 window ending time may be set to the latest test threshold crossing plus a first increment. When the PPIs correspond to a relatively slower atrial rate, e.g., less than 60, 55 or 50 bpm, processor 52 may set the maximum A3 window ending time to the latest test threshold crossing time plus a second increment that is less than the first increment. Processor 52 may set the minimum A3 window ending time to a first, larger decrement less than latest test threshold crossing time when the atrial rate is relatively fast and to a second smaller decrement less than the latest test threshold crossing time when the atrial rate is relatively slow. If the atrial rate is fast, the A3 window ending time range may be set to have a minimum that defines a narrow range of A3 window ending times less than the A3 window ending time established at block 612 because there is less room for the atrial rate to increase. The maximum A3 window ending time may define a wider range of A3 window ending times greater than the established A3 window ending time (because there is more room for the atrial rate to decrease). If the atrial rate is slow, the A3 window ending time range may be set to have a minimum that defines a wider range of A3 window ending times less than the A3 window ending time established at block 612 (because there is more room for the atrial rate to increase) and a maximum that defines a narrower range of A3 window ending times greater than the established A3 window ending time.

Multiple atrial rate ranges may be defined with corresponding increments and decrements applied to the latest threshold crossing times for setting the A3 window ending time range. In an illustrative example, if the atrial rate determined from the PPIs is in a fast range, e.g., greater than 85 bpm, the A3 window ending time range can be established as the A3 window ending time established at block 612 minus 25 ms to the A3 window ending time plus 75 ms. If the atrial rate is moderate, e.g., between greater than 60 bpm but less than or equal to 85 bpm, the A3 window ending time range may be set to be the established A3 window ending time ±50 ms. If atrial rate is in a slow range, e.g., less than or equal to 60 bpm, then the A3 window ending time range may be set to the established A3 window ending time minus 75 ms to the A3 window ending time plus 25 ms.

At block 614, processor 52 may obtain maximum motion signal amplitudes during the PVABPs of the accepted LD P-wave cycles for establishing the PVABP at block 616 to be used by pacemaker control circuit 206 in sensing A4 events. The maximum amplitude of the motion signal sensed during the PVABP of LD P-wave cycles is expected to correspond to the A1 and/or A2 events. In some examples, processor 52 obtains the maximum amplitude of the selected sensing vector signal during the last 50 to 200 ms of the PVABP. The maximum amplitude near the end of the PVABP may be used for determining whether a shorter PVABP can be used. Processor 52 may compare the maximum amplitude of the motion signal during the PVABP or a portion thereof to a threshold amplitude at block 616. The threshold amplitude may be a specified amplitude, determined as a percentage of the early A4 sensing threshold amplitude or set as another threshold amplitude.

If the maximum amplitude of the motion signal determined during the PVABP (or specified portion thereof) is greater than the threshold amplitude, processor 52 may establish the PVABP as a specified time interval longer than the current PVABP. In other examples, the PVABP may be established at block 616 by setting the PVABP equal to the sample time of the maximum amplitude during the current PVABP or portion thereof plus a specified time interval, e.g., plus 50 to 400 ms. In still other examples, processor 52 may determine the latest sample time during the current PVABP that the motion signal is still equal to or greater than the early A4 threshold amplitude (or another specified threshold amplitude). Processor 52 may establish the PVABP at block 616 to a specified time interval plus the latest sample time that the motion signal is still equal to or greater than the threshold amplitude.

When the motion signal amplitude is less than a threshold amplitude, e.g., less than 20% to 80% of the early A4 sensing threshold amplitude during the last X milliseconds of the current PVABP, processor 52 may establish the PVABP for use by pacemaker control circuit 206 to be a specified time interval shorter than the current PVABP. For example, a default PVABP may be 500 to 700 ms in duration or about 600 ms in an example. When the motion signal amplitude during the last 100 ms (or other ending time interval) is less than 50% (or another percentage) of the early A4 sensing threshold amplitude established at block 608, processor 52 may establish the PVABP to be 50 to 100 ms less than the default PVABP. For example, the PVABP may be established to be 600 ms instead of a default PVABP of 650 ms at block 616 when the motion signal amplitude is less than a threshold amplitude for an ending time interval of the default PVABP. In another example, the PVABP may be set to a default or nominal setting of 550 ms and may be increased to 600 ms if the motion signal amplitude is greater than a threshold amplitude during an ending time interval of the 550 ms PVABP. If the motion signal amplitude is equal to or greater than the threshold amplitude during the ending time interval of the default PVABP, processor 52 may establish the PVABP to be the default PVABP or the default PVABP plus a specified time interval, e.g., plus 50 ms.

FIG. 11 is a flow chart 700 of a method for fine tuning an ending time of the A3 window (e.g., ending time 422 of A3 window 424 shown in FIG. 5). Processor 52 may perform the process of flow chart 700 after completing the process of FIG. 10 as indicated by connector “B.” However, it is to be understood that the process of FIG. 11 may be optional. Processor 52 may establish an A3 window ending time and/or A3 window ending time range as described above without proceeding to the flow chart of 700.

In other examples, the A3 window ending time may be set to a default ending time during the process of FIG. 10 or programmed by a user. Processor 52 may establish the A3 window ending time range as described above in conjunction with FIG. 10. A default A3 window ending time may be 700 to 1000 ms after the most recent ventricular event. The A3 window duration may be 150 to 500 ms, as examples, depending on the established PVABP. In an illustrative example, the PVABP may be 600 ms and the A3 window ending time may be 900 ms so that the A3 window is 300 ms in duration. Processor 52 may fine tune the A3 window ending time from a default or user programmed value to an optimized A3 window ending time (which may be within an established A3 window ending time range) according to the process of flow chart 700.

At block 701, processor 52 may receive a signal episode including the motion signal sensed from the selected sensing vector and at least one ECG (and/or EGM) signal. The signal episode is sensed during the non-atrial tracking pacing mode, e.g., the VDI pacing mode. At block 702 the established control parameters may be applied to the motion signal for sensing A4 events from the motion signal episode. At block 704, processor 52 identifies the LD P-wave cycles in the signal episode based on the output of cardiac signal analyzer 51. As described above, additional signal episodes may be acquired if less than a threshold number of LD P-wave cycles are identified during the signal episode.

For at least one of the LD P-wave cycles, processor 52 may determine the latest test threshold crossing time during the A3 window. The A3 window may have an ending time established according to the process of FIG. 10 or set to a default ending time. The test threshold crossing time is set to a percentage of the late A4 sensing threshold amplitude. Processor 52 may update a median or other representative value of the latest test threshold crossing times in the A3 window after every predetermined number of LD P-wave cycles, e.g., after every 3, 5, 6, 8 or other selected number of LD P-wave cycles. In other examples, processor 52 determines a median or other representative value of the latest test threshold crossings at the end of the signal episode.

At block 706, processor 52 may update the A3 window ending time when the median latest test threshold crossing time is updated, e.g., after a specified number of LD P-wave cycles. The A3 window ending time may be increased or decreased by an adjustment interval, e.g., 10 ms, 20 ms, 30 ms or 50 ms toward the median latest test threshold crossing time or toward a target ending time based on the median latest test threshold crossing time. To illustrate, if the A3 window ending time starts at a default value of 850 ms and the median latest test threshold crossing is 750 ms, the A3 window ending time may be decreased by the adjustment interval, e.g., by 20 ms, toward the median latest test threshold time, but not less than a minimum A3 window ending time established during the process of flow chart 600. It is to be understood that the updating at block 706 can include no adjustment to the A3 window ending time when the A3 window ending time is equal to (or within the adjustment interval of) the median latest test threshold crossing time or a target ending time based on the median latest test threshold crossing time.

If additional cycles remain in the signal episode, as determined by processor 52 at block 710, after updating the A3 window ending time, processor 52 may return to block 704. When all cycles have been evaluated for the current signal episode, processor 52 may determine if the A3 window ending time is stable at block 708. If the A3 window ending time has reached a target ending time based on the median latest test threshold crossing time (or reached a minimum or maximum A3 window ending time) and remained at the same ending time for one or more updates at block 706, processor 52 may determine that the A3 window ending time is stable at block 708.

If the A3 window ending time, however, is still being progressively incremented or decremented toward a target A3 window ending time when updated at block 706, processor 52 may determine that the A3 window ending time has not reached a stable value at block 708. Processor 52 may return to block 701 to obtain another signal episode to continue the process of fine tuning the A3 window ending time. Processor 52 may obtain a maximum number of signal episodes, e.g., two to five signal episodes each being 3 to 30 cardiac cycles in length as examples, for updating the A3 window ending time. When the A3 window ending time reaches a stable value, as determined at block 708, processor 52 determines that the A3 window ending time is established at the current value at block 710.

Processor 52 may transmit the established control parameters (e.g., motion signal sensing vector, early A4 sensing threshold amplitude, late A4 sensing threshold amplitude, A3 window ending time range, and/or A3 window ending time) to pacemaker 14 for application during AVS pacing. At block 712, pacemaker 14 may switch from the VDI pacing mode to a VDD pacing mode, which may be in response to a programming command, and apply the established control parameters received from external device 50 for controlling AVS pacing pulses during the VDD pacing mode.

FIG. 12 is a flow chart 750 of a method that may be performed by processing circuitry of medical device system 10 for establishing AVS pacing control parameters according to another example. In some examples, in addition to or alternatively to establishing control parameters relating to A4 event sensing, processor 52 may establish at least one AVS pacing control parameter based on the atrial rate corresponding to the P-wave timing markers output by cardiac signal analyzer 51. Setting one or more AVS pacing control parameters based on the atrial rate can promote reliable tracking of A4 events during AVS pacing.

At block 752, processor 52 may determine the PPIs between the P-wave timing markers output by cardiac signal analyzer 51 when one or more signal episodes are received by cardiac signal analyzer 51 as input. Processor 52 may determine a representative value of the PPIs, e.g., a mean or median PPI, and a corresponding atrial rate in some examples. The atrial rate may be an intrinsic atrial rate and may be an indication of the patient's normal resting sinus rate. Blocks 754, 756 and 758 refer to different AVS pacing control parameters that may be established by processor 52 based on the determined PPIs.

For example, at block 754, processor 52 may set a maximum upper tracking rate based on the PPIs. The maximum upper tracking rate is the maximum rate of sensed A4 events that the pacemaker control circuit 206 will track for delivering AVS pacing pulses during the atrial synchronous pacing mode. When an A4 event is sensed such that the triggered AVS pacing pulse scheduled at the AV pacing interval will occur at a ventricular rate (from a most recent preceding ventricular pacing pulse or intrinsic R-wave) that is faster than the maximum upper tracking rate, control circuit 206 may withhold the triggered AVS pacing pulse. Control circuit 206 may switch to a non-atrial tracking ventricular pacing mode when sensed A4 events are occurring faster than a maximum upper tracking rate. Control circuit 206 may remain in the non-atrial tracking pacing mode until pacing mode switching criteria are met, e.g., until the atrial rate has decreased.

The upper tracking rate may be set to be relatively higher when the patient's atrial rate is relatively fast. For example, if the PPIs correspond to an atrial rate of 80 bpm or faster processor 52 may set the maximum tracking rate to a rate that is higher than the atrial rate, e.g., 10 to 30 bpm faster or up to a maximum tracking rate of about 110 to 120 bpm. When the patient's atrial rate is relatively slower, e.g., less than 80 bpm based on the determined PPIs, processor 52 may set the maximum tracking rate to a slower rate, e.g., 80 to 100 bpm. The maximum tracking rate may be set to a default rate, e.g., 100 bpm, unless the PPIs indicate a relatively fast atrial rate, e.g., 80 bpm or faster, which can occur in patients in the first days or weeks after surgical implant of pacemaker 14.

At block 756, processor 52 may establish a rate smoothing increment based on the PPIs. Control circuit 206 may set a rate smoothing interval (RSI) based on recent ventricular cycle lengths (VCLs) determined between consecutive ventricular events during AVS pacing. For example, a paced VCL may be determined as the actual ventricular rate interval between two consecutive AVS pacing pulses or between a non-AVS pacing pulse and an AVS pacing pulse or between two consecutive non-AVS pacing pulses that may be delivered at an RSI instead of the LRI. The RSI may be started in response to each ventricular event, e.g., each AVS pacing pulse, sensed R-wave, or non-AVS pacing pulse, during AVS pacing. When an A4 event is not sensed before the RSI expires, pulse generator 206 may deliver a ventricular pacing pulse. The RSI maintains the ventricular rate near the AVS paced rate when an A4 event is not sensed during one or more cardiac cycles to avoid an abrupt change in the ventricular rate. The RSI may gradually be adjusted by pacemaker control circuit 206 toward the LRI corresponding to the programmed ventricular pacing lower rate when the A4 event is not sensed for multiple ventricular cycles.

In some examples, control circuit 206 determines a rate smoothing base interval (RSBI) from the most recent paced VCL. The RSBI may be initialized to the programmed LRI. The RSBI may be compared to the next paced VCL determined by control circuit 206 as the time interval between two consecutive pacing pulses, which may be delivered as AVS pacing pulses or pacing pulses scheduled at RSIs. If the RSBI is greater than the next paced VCL, the RSBI is decreased by an adjustment interval, e.g., by 8 to 20 ms. If the RSBI is less than the next paced VCL, it may be increased by the adjustment interval. If the RSBI is equal to (or within an adjustment interval) of the most recent paced VCL, it is not adjusted by control circuit 206 and remains at its current value. In this way, control circuit 206 may update the RSBI on each paced VCL to track the actual paced ventricular rate on a beat by beat basis. The RSBI may be adjusted up or down by a relatively small adjustment interval, e.g., 8 to 20 ms, based on the actual VCL(s), so that the RSBI trends toward and closely follows the actual paced VCLs.

The RSI may be determined by control circuit 206 as the RSBI plus a smoothing increment. The smoothing increment may be established at block 756 by processor 52 based on the determined PPIs. For example, the smoothing increment may be set to a relatively long increment, e.g., 100 to 200 ms, when the atrial rate is relatively slow (e.g., 80 bpm or less) based on the PPIs. The rate smoothing increment may be set by processor 52 to be a relatively short increment, e.g., 25 to 75 ms, when the atrial rate is relatively fast based on the determined PPIs, e.g., greater than 80 bpm.

At block 758, processor 52 may establish the ventricular pacing lower rate based on the PPIs. The pacing lower rate may be set to a relatively faster rate when the PPIs correspond to a relatively fast atrial rate or to a relatively slower rate when the PPIs correspond to a relatively slow atrial rate. A patient having an intrinsic resting atrial rate that is relatively fast may need faster ventricular pacing rate support than a patient having a relatively slower intrinsic atrial rate. Processor 52 may establish the pacing lower rate to be 10 to 30 bpm slower than the atrial rate corresponding to the PPIs, for example. If the ventricular pacing lower rate is faster than or nearly equal to a patient's normal resting atrial rate, asynchronous ventricular pacing pulses may be delivered more frequently than AVS pacing pulses because the LRI may expire before an A4 event is sensed. Accordingly, by setting the ventricular pacing lower rate to be less than the atrial rate based on the determined PPIs, processor 52 may establish an AVS pacing control parameter that promotes AVS pacing for the patient but provides sufficient ventricular rate support for the patient when A4 events are not being sensed.

While the flow chart 750 depicts setting the upper tracking rate, rate smoothing increment and the ventricular pacing lower rate based on determined PPIs, it is to be understood that one or more of these AVS pacing control parameters (or none) may be established based on the PPIs by processor 52 in various examples. Furthermore, while shown being established in a particular order, the upper tracking rate, rate smoothing increment and/or ventricular pacing lower rate may be established based on determined PPIs in any order or combination.

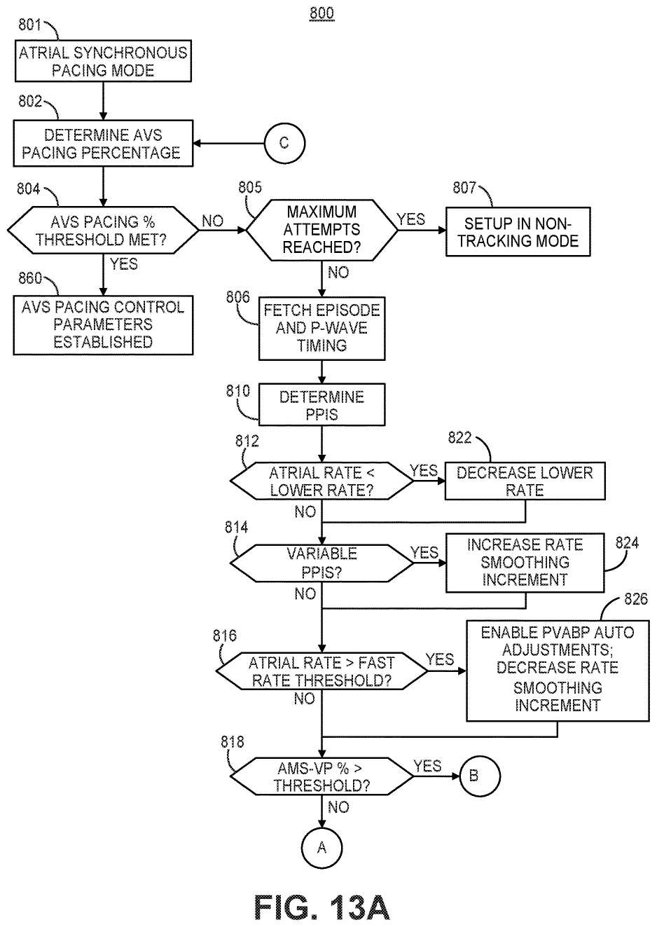

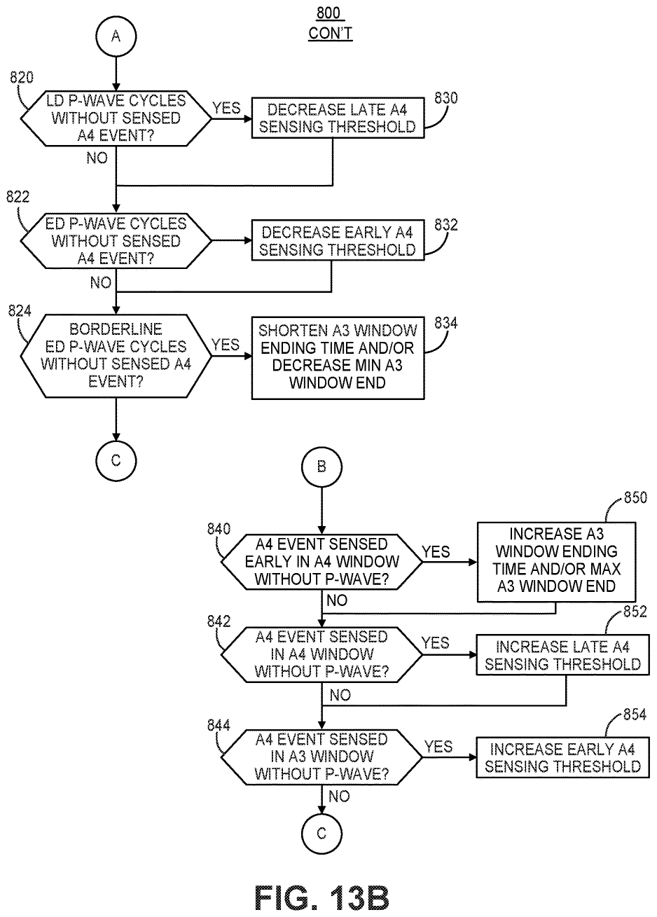

FIGS. 13A and 13B depict a flow chart 800 of a method that may be performed by processing circuitry of the medical device system 10 of FIG. 1 according to another example. For the sake of convenience, the process of FIGS. 13A and 13B is described as being performed by external device processor 52. It is to be understood, however, that pacemaker control circuit 206 may perform any or all of the process of flow chart 800. After establishing AVS pacing control parameters according to any of the examples or combinations of examples described above, processor 52 may perform the process of flow chart 800 for verifying that the established parameters result in a high percentage of AVS pacing pulses during a subsequent signal episode. At block 801, if not already operating in an atrial synchronous pacing mode, pacemaker 14 may switch the pacing mode from the asynchronous (e.g., VDI) pacing mode to an atrial synchronous (e.g., VDD) pacing mode for delivering AVS pacing. Pacemaker 14 may switch to the VDD pacing mode, for example, automatically after transmitting signal episode data to external device 50 or in response to receiving a pacing mode command from external device 50. External device 50 may transmit the established AVS pacing control parameters determined according to any of the above techniques to pacemaker 14. Control circuit 206 may apply the established AVS pacing control parameters for operating in the VDD pacing mode, e.g., for controlling A4 event sensing and for controlling the timing of ventricular pacing pulse delivery. In some examples, any of the AVS pacing control parameters may be programmed by a user without necessarily performing the techniques described above for establishing a starting value.

During the verification process of flow chart 802, telemetry circuit 208 of pacemaker 14 may transmit the EGM signal, motion signal, sensed A4 event markers, delivered ventricular pacing markers, sensed R-wave markers and/or related data to processor 52 to provide processor 52 with the data necessary to verify that the established AVS pacing control parameters are causing pacemaker 14 to perform as expected. For example, pacemaker 14 may be expected to deliver at least a threshold percentage of AVS pacing pulses out of all ventricular events, paced and sensed, during operation in the atrial synchronous pacing mode according to the established AVS pacing control parameters. For example, in a patient having complete AV block, AVS pacing pulses that are delivered following a true P-wave and subsequently sensed A4 event signal may be expected to be at least 70%, 80%, 90% or a higher percentage of all ventricular events occurring in a specified time period, which may include any intrinsic R-waves sensed by sensing circuit 204 and all delivered ventricular pacing pulses (delivered at an AV interval, an LRI, an RSI, etc.).

During an atrial synchronous ventricular pacing episode, control circuit 206 or processor 52 may determine the AVS pacing pulse percentage (out of all ventricular events) at block 802. The AVS pacing pulse percentage can be determined by processor 52 using the P-wave timing output of cardiac signal analyzer 51 to determine the true AVS pacing percentage. The true AVS pacing percentage is the percentage of pacing pulses delivered at an AV interval from a sensed A4 event that follows a P-wave timing marker within a maximum expected electromechanical delay, e.g., 100 ms or less or 150 ms or less. Control circuit 206 may be configured to track the percentage of atrial mechanical sense to ventricular pacing pulses (AMS-VP pacing pulses) delivered by pulse generator as the pacing pulses delivered at an AV interval following a sensed A4 event (atrial mechanical sense or “AMS”). Processor 52 may be configured to determine how many of the ventricular pacing pulses delivered as AMS-VP pacing pulses are associated with a leading P-wave timing marker. For instance, processor 52 may determine what percentage of all ventricular events (or all ventricular pacing pulses) are ventricular pacing pulses delivered upon expiration of an AV interval (as AMS-VP pulses) and within a maximum AVS time interval from a preceding P-wave. In an illustrative example, processor 52 may determine the percentage of ventricular pacing pulses that are delivered within 300 ms or other specified maximum AVS time interval from a P-wave timing marker and at an AV interval following a sensed A4 event. This true AVS pacing percentage may be determined from a specified number of ventricular events, e.g., 10 to 100 ventricular events, or from a specified time interval, e.g., 10 seconds to 2 minutes.

Processor 52 may compare the true AVS pacing pulse percentage to a threshold percentage at block 804. If the threshold percentage is met, processor 52 may determine that the process for establishing AVS pacing control parameters is complete at block 860. Pacemaker 14 may continue operating in the atrial synchronous ventricular pacing mode with the established AVS pacing control parameters in effect. It is to be understood that the control parameters may be subsequently adjusted by control circuit 206, e.g., according to any of the techniques disclosed in the above-incorporated U.S. patent application Ser. No. 17/159,596 (Sheldon, et al.) and U.S. patent application Ser. No. 17/159,635 (Sheldon, et al.).

When the AVS pacing percentage is less than a threshold percentage at block 804, and a maximum number of attempts to achieve the desired AVS pacing percentage has been reached (decision block 805), processor 52 may advance to block 807 to perform, in cooperation with pacemaker 14, the setup procedure for re-establishing starting values of one or more AVS pacing control parameters. The maximum number of attempts may be 1 to 5 attempts as examples. When one or more adjustments to AVS pacing control parameters during the atrial synchronous pacing mode do not yield the desired AVS pacing percentage as verified by the relative timing of identified P-waves and delivered ventricular pacing pulses, control circuit 206 may switch back to a non-atrial tracking pacing mode, e.g., a VDI pacing mode, to re-evaluate the cardiac signals and re-establish starting AVS pacing control parameters at block 807, e.g., according to any of the techniques described above. In some examples, processor 52 generates a user notification for display by display unit 54 prompting the user to manually restart the set-up procedure, which may proceed automatically once an auto setup command is entered by a user.

When the maximum number of attempts has not been reached at block 805, processor 52 may fetch a signal episode and obtain the P-wave timing marker output of cardiac signal analyzer 51 for one or more cardiac cycles in the signal episode at block 806. The signal episode may be fetched by sending a request command to pacemaker 14 to transmit a motion signal episode and/or related data and recording at least one ECG signal sensed concurrently with the motion signal episode to obtain the same ventricular cycles in the motion signal and the ECG signal. Cardiac signal analyzer 51 may receive the at least one ECG signal input and outputs the P-wave timing markers of identified P-waves in the signal episode. As described above, instead of or in addition to an ECG signal, processor 52 may receive an EGM signal from pacemaker 14 to be provided as input to cardiac signal analyzer 51.

Processor 52 may perform an analysis of the signal episode and P-wave timing markers for troubleshooting a low AVS pacing percentage and making an adjustment to one or more AVS pacing control parameters. In some instances, the analysis includes determining PPIs at block 810 for use in identifying atrial rate related causes of a low AVS pacing percentage. At block 812, processor 52 may determine PPIs from the output of cardiac signal analyzer 51 and determine an associated atrial rate. Processor 52 may compare the atrial rate to the ventricular pacing lower rate (or compare PPIs to the LRI corresponding to the ventricular pacing lower rate). If the atrial rate is slower than the ventricular pacing lower rate (or PPIs are longer than the LRI), ventricular pacing at the LRI may be precluding A4 event sensing because the ventricular pacing pulse is delivered before an A4 event is sensed, resulting in a low AVS pacing pulse percentage. In response to determining that one or more PPIs are longer than the LRI, processor 52 may decrease the ventricular pacing lower rate at block 822 to be less than the atrial rate corresponding to the determined PPIs.

At block 814, processor 52 may determine if the determined PPIs are variable by applying interval variability criteria to the PPIs. Processor 52 may determine a metric of PPI variability by determining a standard deviation, variance, range, beat-to-beat difference, and/or other measure of spread or beat-to-beat variability of the PPIs. If a metric of variability of the PPIs is greater than a threshold, processor 52 may determine that variable PPIs associated with a varying atrial rate may be causing intermittent sensing or undersensing of A4 events. When the atrial rate increases, causing the actual VCLs to shorten, the RSI is shortened by control circuit 206 to provide a smooth ventricular rate transition if an A4 event is not sensed. If the atrial rate slows again faster than the RSI is adjusted by control circuit 206, a ventricular pacing pulse may be delivered at the RSI prior to an A4 event, preventing sensing of the A4 event. Accordingly, processor 52 may increase the rate smoothing increment at block 824 when PPI variability is detected. By increasing the rate smoothing increment, a longer RSI promotes a longer A4 window and greater opportunity for sensing A4 events when the PPI increases and decreases, promoting sustained A4 event sensing during variation of the atrial rate. Methods for controlling the RSI by pacemaker 14 that may be implemented in conjunction with the techniques disclosed herein are generally described in U.S. Patent Application Publication No. 2019/0321634 (Sheldon, et al.).

At block 816, processor 52 may determine if the atrial rate is faster than a fast rate threshold. The fast rate threshold may be 90 bpm, 95 bpm, 100 bpm or other selected rate threshold. Processor 52 may determine the PPIs from the signal episode and determine if a predetermined percentage of the PPIs are shorter than a threshold interval corresponding to the fast rate. In other examples, processor 52 may determine if the mean, median, minimum, maximum or other representative PPI of the PPIs determined from the signal episode is shorter than a threshold interval corresponding to the fast rate threshold. When the atrial rate corresponding to the PPIs determined from the signal episode is faster than the rate threshold, processor 52 may enable automatic PVABP adjustment and/or decrease the rate smoothing increment at block 826.

When the atrial rate is faster than the rate threshold at block 816, A4 events may occur at relatively shorter time intervals after the ventricular events during an AVS rhythm. Pacemaker control circuit 206 may be configured to automatically adjust the PVABP between a minimum PVABP and maximum PVABP based on actual VCLs which may be determined between consecutive ventricular events, e.g., consecutive AVS or non-AVS pacing pulses. When the PVABP is decreased to the minimum PVABP by control circuit 206 in response to VCLs that are shorter than the threshold interval corresponding to the fast rate threshold, the A3 window starts earlier in the ventricular cycle. When the PVABP is increased to the maximum PVABP by control circuit 206 in response to VCLs that are longer than the threshold interval corresponding to the fast rate threshold, the A3 window starts later in the ventricular cycle. By enabling automatic PVABP adjustment by control circuit 206 at block 826, A4 event tracking may be improved during relatively fast atrial rates. Methods for adjusting the PVABP that may be implemented in conjunction with the techniques disclosed herein are generally described in provisional U.S. patent application Ser. No. 63/274,323 (Sheldon, et al.).

Processor 52 may additionally or alternatively decrease the rate smoothing increment at block 826 in response to determining that the atrial rate based on PPIs is faster than the fast rate threshold. By decreasing the rate smoothing increment, the RSI is adjusted by a smaller percentage of the current RSI each time it is adjusted. In this way, pacing at the RSI continues to approximately match the PPIs occurring during a relatively fast atrial rate to promote recovery of A4 event sensing when the A4 event is not sensed for one or more ventricular cycles. By changing the RSI more gradually using a smaller rate smoothing increment, the timing of the A4 events in each ventricular cycle is expected to change less from beat to beat, assuming the atrial rate is stable, enabling atrial event detector circuit 240 a better chance to regain A4 event sensing after a missed A4 event.

It is to be understood that any time that one or more adjustments are made to the AVS pacing control parameters, e.g., at any of blocks 822, 824 or 826, processor 52 may return to block 802 to re-determine the true AVS pacing percentage and determine if it has improved to be greater than a threshold percentage at block 804 subsequent to the adjustment(s). However, for the sake of simplicity, the process of flow chart 800 is shown to proceed to block 818 for checking for possible A4 event undersensing or oversensing that may be causing a low AVS pacing percentage.

At block 818, processor 52 may determine the AMS-VP percentage of all ventricular events (or of all paced ventricular events) during the specified time interval. When the true AVS pacing percentage is less than a threshold percentage, the AMS-VP percentage (ventricular pacing pulses delivered at an AV interval from a sensed A4 event) may be the same or different than the true AVS pacing percentage. When the AMS-VP percentage is less than or equal to a threshold percentage at block 818, processor 52 may perform an analysis of the cardiac signals and events for identifying A4 event undersensing and taking a corrective action. The threshold percentage applied at block 818 may be the same threshold percentage applied at block 804 or a different percentage that allows for some undersensing (or some oversensing of A4 events). An example process of identifying A4 event undersensing and adjusting AVS pacing control parameters is shown in FIG. 13B (following the path of connector “A” in flow chart 800).

In some instances, the AMS-VP percentage is greater than the threshold percentage at block 818. The AMS-VP percentage may be higher than the true AVS percentage when one or more ventricular pacing pulses triggered by a sensed A4 event do not follow an identified P-wave within the maximum expected electromechanical delay. In this case, oversensing of false A4 events may be occurring (e.g., due to noise in the motion signal, oversensing of A3 events as false A4 events, etc.). An example process for troubleshooting oversensing of A4 events, e.g., when the AMS-VP percentage is greater than the true AVS pacing percentage, is described below in conjunction with FIG. 13B (following the path of connector “B” in flow chart 800).

To facilitate the processes relating to identifying undersensing and/or oversensing of A4 events, cardiac cycles of the signal episode during the AVS verification process of flow chart 800 may be classified by processor 52 as ED P-wave cycles when a P-wave timing marker output of cardiac signal analyzer 51 falls in the A3 window extending from the expiration of the established PVABP to the established A3 window ending time. Ventricular cycles of the signal episode may be classified as LD P-wave cycles when the P-wave timing marker output of cardiac signal analyzer 51 is after the A3 window ending time, during the A4 window and prior to the next ventricular event that starts the next ventricular cycle.

Referring to FIG. 13B, which is a continuation of flow chart 800 from FIG. 13A, in response to determining that the AMS-VP percentage is less than a threshold percentage at block 818 (FIG. 13A), processor 52 may determine if cardiac cycles during the signal episode are LD P-wave cycles at block 820. If so, processor 52 may determine at block 820 if one or more LD P-wave cycles are identified without an A4 event sensed by control circuit 206 during the A4 window (after the P-wave) or within a maximum expected electromechanical delay after the P-wave, e.g., within 200 ms, 150 ms, 100 ms or other maximum expected electromechanical delay. When processor 52 determines that A4 events are not sensed in the A4 window following a P-wave in one or more LD P-wave cycles during the signal episode, processor 52 may decrease the late A4 sensing threshold at block 830. A4 events occurring in the A4 window may be undersensed due to the late A4 sensing threshold being too high, and therefore resulting in fewer AVS pacing pulses than expected (e.g., less than a threshold percentage of AVS pacing pulses) during the signal episode.

At block 822, processor 52 may determine if cardiac cycles are determined to be ED P-wave cycles without an A4 event sensed by control circuit 206 during the A3 window (or within a maximum expected electromechanical delay from an identified P-wave) of one, some or all of the ED P-wave cycles. An ED P-wave cycle may be identified when cardiac signal analyzer 51 outputs a P-wave timing marker during the A3 window ending time. If a P-wave timing marker indicates that a P-wave is identified during the A3 window but an A4 event is not sensed following the P-wave, processor 52 may identify A4 undersensing at block 822. In response to one or more cardiac cycles of the signal episode being identified as ED P-wave cycles with A4 undersensing, processor 52 may decrease the early A4 sensing threshold amplitude at block 832. If ED P-wave cycles occur during the signal episode without corresponding sensed A4 events, A4 event undersensing due to the early A4 sensing threshold amplitude applied during the A3 window being too high may be causing the low AVS pacing percentage.

At block 824, processor 52 may determine if borderline ED P-wave cycles are present in the signal episode. A borderline ED P-wave cycle may be identified by processor 52 as a cardiac cycle with the P-wave timing near the ending time of the A3 window, e.g., within the last 50 to 100 ms of the A3 window. If borderline ED P-wave cycles are present, processor 52 may further determine if A4 events are sensed following the P-wave in or after the A3 window in the borderline ED P-wave cycles. When A4 events are not sensed in one or more borderline ED P-wave cycles, A4 undersensing may be contributing to the low AVS pacing percentage due to the A3 window ending time being too long. In response to identifying one or more borderline ED P-wave cycles with A4 undersensing, processor 52 may shorten the A3 window ending time at block 834. The A3 window ending time may be shortened by a predetermined decrement, e.g., 25 to 100 ms, or to a previously established minimum A3 window ending time.

In some examples, the A3 window ending time may be shortened based on the latest borderline ED P-wave timing marker, e.g., 50 to 100 ms earlier than an ED P-wave timing marker. Additionally or alternatively, processor 52 may decrease the maximum A3 window ending time of the range of A3 window ending times that the control circuit 206 may adjust the A3 window ending time between. The maximum A3 window ending time may be decreased to be 50 to 100 ms greater than the shortened A3 window ending time. If the A3 window ending time is not shortened, the maximum A3 window ending time may be decreased, e.g., by 25 to 50 ms from the current setting.

After making one or more adjustments at any of block 830, 832 and/or 834, processor 52 may return to block 802 (FIG. 13A) as indicated by connector “C” to determine if the adjustment(s) improve the true AVS pacing percentage. In other examples, processor 52 may advance to block 840 of flow chart 800 to analyze the signal episode for possible A4 oversensing and making corrective adjustments to the AVS pacing control parameters.

At block 840, processor 52 may determine if an A4 event is sensed in the A4 window, near the end of the A3 window, during one or more cardiac cycles. An A4 event sensed within the first 50 to 100 ms of the A4 window may be an oversensed signal. If one or more A4 events are sensed early in the A4 window of one or more cardiac cycles without a preceding P-wave within the A3 window or the A4 window, e.g., within a threshold time interval earlier than the sensed A4 event, the A4 event may be oversensed due to the A3 window ending time being too short. Processor 52 may increase the A3 window ending time at block 850.

The A3 window ending time may be increased by a predetermined increment, e.g., 25, 50, 75 or 100 ms. The A3 window ending time may be increased to a maximum A3 window ending time. The A3 window ending time may be adjusted based on the timing of the identified oversensed A4 events. For example, the A3 window ending time may be set to be longer than the timing of a latest oversensed A4 event identified in the signal episode. Additionally or alternatively, the maximum A3 window ending time may be increased by processor 52 at block 850. The maximum A3 window ending time may be increased by 25 to 100 ms, as examples. The maximum A3 window ending time may be increased to be 50 to 100 ms longer than the A3 window ending time that is increased at block 850. The maximum A3 window ending time may be increased based on the timing of an oversensed A4 event. For example, the maximum A3 window ending time may be set by processor 52 to be longer than the latest oversensed A4 event occurring early in the A4 window that is identified from the signal episode.

At block 842, processor 52 may identify any cardiac cycles during the signal episode that have an A4 event sensed from the motion signal during the A4 window of the cardiac cycle (but later than the early portion of the A4 window analyzed at block 840). Processor 52 may determine if a cardiac cycle having an A4 event sensed during the A4 window is not identified as an LD P-wave cycle or does not include an identified P-wave within a threshold time interval (e.g., maximum expected electromechanical delay) prior to the A4 event. For example, if an A4 event is sensed during the A4 window but no P-wave is identified within 100 to 200 ms prior to the A4 event, the A4 event is likely an oversensed event. Processor 52 may identify an A4 event sensed during the A4 window of a cardiac cycle without a P-wave identified during the A4 window (or within a threshold time interval prior to the A4 event) as an oversensed A4 event at block 842. The A4 event oversensed in the A4 window may occur when the late A4 sensing threshold amplitude is too low. In response to identifying one or more oversensed A4 events occurring during one or more A4 windows of the signal episode, processor 52 may increase the late A4 sensing threshold at block 852.

It is to be understood that if an oversensed A4 event is identified at block 840 during the early portion of the A4 window and the A3 window ending time or maximum A3 window ending time is increased at block 850, processor 52 may or may not increase the late A4 sensing threshold based on the same oversensed A4 event identified in the early portion of the A4 window. In some examples, processor 52 may increase the A3 window ending time at block 850 when an oversensed A4 event is in the early portion of the A4 window without adjusting the late A4 sensing threshold amplitude at block 852. Processor 52 may increase the A4 sensing threshold amplitude at block 852 in response to identifying an A4 event in the later portion of the A4 window without adjusting the A3 window ending time at block 850.

At block 844, processor 52 may determine if A4 events are being sensed in the A3 window during one or more cardiac cycles that are not ED P-wave cycles. If so, the A3 event corresponding to passive ventricular filling may be oversensed as a false A4 event due to the early A4 sensing threshold amplitude being too low. Processor 52 may identify an oversensed A4 event in an A3 window when a P-wave is not identified by cardiac signal analyzer 52 during the A3 window (or within a threshold time interval prior to the sensed A4 event). Oversensing of A4 events may lead to a high percentage of AMS-VP pacing pulses, e.g., greater than the true AVS pacing percentage. Processor 52 may increase the early A4 sensing threshold at block 854 in response to identifying A4 event oversensing in the A3 window of one or more non-ED P-wave cycles.

After assessing any of the conditions represented by blocks 820, 822 and/or 824 that could be causing the percentage of true AVS pacing pulses to be less than expected due to A4 event undersensing and/or assessing any of the conditions of blocks 840, 842, 844 that could be causing the percentage of true AVS pacing pulses to be less than expected due to A4 event oversensing, and making any corresponding AVS control parameter adjustments as needed at respective blocks 830, 832, 834, 850, 852 and/or 854, processor 52 may return to block 802 (FIG. 13A) as indicated by connector “C.” The AVS pacing percentage may be redetermined while pacemaker 14 continues to operate in the atrial synchronous pacing mode but with any AVS pacing control parameters adjustments in effect.

If the true AVS pacing percentage is still less than the threshold percentage at block 804, another signal episode may be obtained and the process of testing for the conditions relating to atrial rate, possible A4 event undersensing and/or possible A4 event oversensing may be repeated, with additional AVS pacing control parameters being adjusted as needed and/or additional adjustments made to one or more of the same AVS pacing control parameters. In response to the AVS pacing percentage meeting the threshold percentage at block 804, processor 52 may confirm the AVS pacing control parameters as being established at the current settings at block 850. The confirmed, established AVS pacing control parameters may be programmed into pacemaker 14 by external device 50 for use by control circuit 206 in controlling pacemaker operations during the VDD pacing mode.

In FIGS. 13A and 13B, blocks 812, 814, 816, 820, 822, 824, 840, 842 and 844 represent conditions that processor 52 may identify as possible causes of the low AVS pacing pulse percentage identified at block 804. Blocks 810-822 are shown in a particular order. It is to be understood, however, that blocks 812, 814, 816, 820, 822, 824, 840, 842 and 844 may be performed in any order, different than shown in FIGS. 13A-13B, and in some instances some blocks may be left out altogether and/or other conditions that may be causing the low AVS pacing percentage may be tested for. When the result of one of blocks 812, 814, 816, 820, 822, 824, 840, 842 and 844 is affirmative (“yes” branch), flow chart 800 depicts a corresponding adjustment to an AVS pacing control parameter at one of respective block 822, 824, 826, 830, 832, 834, 850, 852, or 854. For the sake of convenience, following an adjustment to an AVS pacing control parameter at one of blocks 822, 824, 826, 830, 832, 834, 850, or 852 flow chart 800 depicts processor 52 advancing to the next one of blocks 814, 816, 820, 822, 824, 840, 842 and 844 to determine if the next condition that may be causing the low AVS pacing percentage is true before redetermining the AVS pacing pulse percentage at block 802.

It is to be understood, however, that in other examples, processor 52 may return to block 802 after a single AVS pacing control parameter adjustment in response to any one of the conditions of blocks 812, 814, 816, 820, 822, 824, 840, 842 and 844 being true. The medical device system 10 may be configured to obtain relatively short signal episodes, e.g., 3 to 60 seconds, after each AVS pacing control adjustment is made one at a time at one of blocks 814, 816, 820, 822, 824, 840, 842 and 844. Processing circuitry of medical device system 10 may be configured to redetermine the AVS pacing percentage after each AVS pacing control adjustment to determine if the adjustment sufficiently improves the AVS pacing percentage to meet the threshold percentage at block 804 before determining if other conditions of decision blocks 812, 814, 816, 820, 822, 824, 840, 842 and/or 844 are true and making other adjustments to other AVS pacing control parameters.

In the example shown, processor 52 can test the current signal episode for multiple possible causes of a low AVS pacing percentage as represented by decision blocks 812, 814, 816, 820, 822, 824, 840, 842 and/or 844 before returning to block 802. As such, processor 52 may be configured to test for any one, some or all of the conditions represented by the decision blocks of 814, 816, 820, 822, 824, 840, 842 and 844 based on one signal episode and provide a corresponding adjustment at a respective one of blocks 822, 824, 826, 830, 832, 834, 850, 852, or 854 for each condition that is determined to be true for that signal episode as needed.

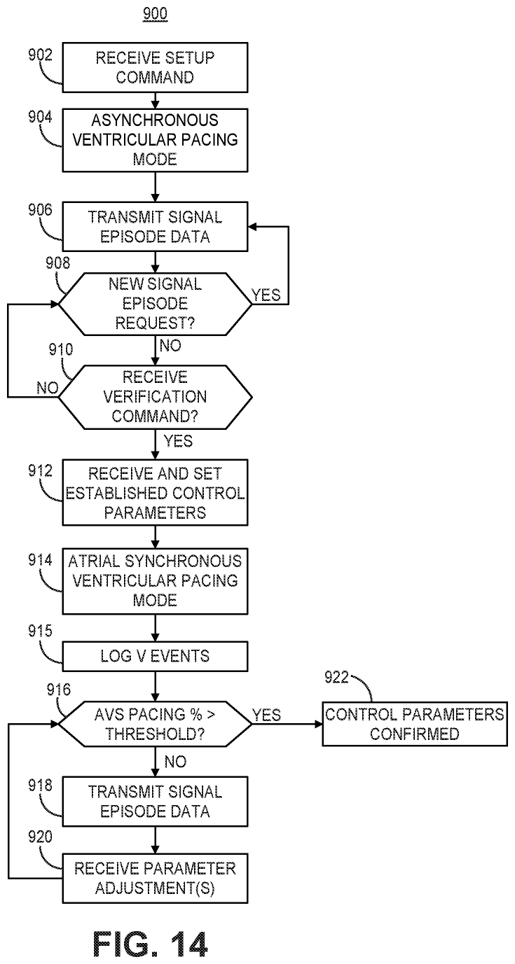

FIG. 14 is a flow chart 900 of a method that may be performed by pacemaker 14 of medical device system 10 of FIG. 1 according to some examples. The process of flow chart 900 may be performed at the time of pacemaker implant and/or during any in-clinic or remote patient follow up procedure. As described above, pacemaker 14 may operate cooperatively with external device 50 to acquire signal episodes while operating according to a ventricular pacing mode with pacing control parameters in effect as needed to enable AVS pacing control parameters to be established and verified by external device 50. Pacemaker 14 may receive a setup command from external device 50 at block 902 via telemetry circuit 208. In response to the setup command, control circuit 206 switches to an asynchronous ventricular pacing mode, e.g., a VDI pacing mode, at block 904 (if not already operating in the asynchronous ventricular pacing mode).

During the VDI pacing mode, control circuit 206 may deliver asynchronous pacing pulses at a programmed lower rate, e.g., 40 to 60 bpm, and sense A4 events according to default atrial event sensing control parameters. The default atrial event sensing control parameters may include, for example, a PVABP of 500 to 650 ms, an A3 window ending time of 750 to 1000 ms, an early A4 sensing threshold amplitude of 2.0 to 2.5m/s², and a late A4 sensing threshold amplitude of 1.0 to 1.5 m/s² as non-limiting illustrative examples. The rate smoothing increment may be set to a default value of 50 to 200 ms, the maximum tracking rate may be set to a default value of 100 to 130 bpm. While atrial event detector circuit 240 may sense A4 events during the VDI pacing mode according to default parameters, control circuit 206 may ignore the A4 events sensed during the asynchronous ventricular pacing mode for the purposes of triggering ventricular pacing pulses. Pacemaker control circuit 206, however, may log sensed A4 event data in memory 210 during the automatic setup procedure. For example, as described below in conjunction with FIG. 15, control circuit 206 may log the A4 event time (corresponding to when the motion signal crosses the A4 sensing threshold amplitude) and the A4 event peak amplitude.

At block 906, control circuit 206 may acquire a signal episode in response to the setup command. The signal episode may extend for a specified time interval (e.g., 5 to 120 seconds long) or include specified number of cardiac cycles of each axis signal received from motion sensor 212. In some examples, the motion sensor 212 is configured to pass a filtered rectified motion signal from each axis individually and each combination of two axis signals and the combination of all three axis signals of a three-dimensional sensor as all of the available sensing vector signals. Control circuit 206 may select which sensing vector signals to receive and transmit as signal episode data to external device 50. In some examples, each single axis signal is transmitted to external device processor 52 so that each signal axis signal can be evaluated by processor 52 alone and/or in one or more two-axis combinations and/or the three-axis combination. In one example, the three possible two-axis combinations are received by control circuit 206 and transmitted to external device processor 52 in the signal episode.

Control circuit 206 may additionally or alternatively determine for each cardiac cycle of the signal episode the motion signal features used by processor 52 for establishing AVS pacing control parameters. However, pacemaker 14 may generally have limited processing power and a limited power supply compared to an external computing device. As such, the signal episode data may be obtained and transmitted to external device 50 in a manner that minimizes the processing required by control circuit 206 during the setup procedure.

Control circuit 206 may transmit a corresponding episode of the sensed EGM signal received from sensing circuit 86 with the signal episode data at block 906. Control circuit 206 may transmit ventricular pacing pulse timing markers and any ventricular sensed event timing markers with the signal episode data. When control circuit 206 is configured to log sensed A4 event data in memory 210, the logged sensed A4 event data (e.g., A4 event time and amplitude) may be transmitted with the signal episode data.

In some examples, control circuit 206 may transmit a signal episode of predetermined duration at block 906 then wait for another episode request at block 908. Subsequent episodes may be requested by external device 50 in order to accumulate a threshold number of accepted cardiac cycles and/or for sequentially establishing multiple AVS pacing control parameters based on analysis of sequentially obtained signal episodes. As such, the new signal episode request at block 908 may include an AVS pacing control parameter established based on a preceding signal episode. Control circuit 206 may put the established control parameter into effect during the ongoing VDI pacing mode operation and transmit the next signal episode at block 906 with the established control parameter in effect. For example, as described above in conjunction with FIGS. 9-11, the motion signal sensing vector, A4 sensing threshold amplitudes, and A3 window ending time may be sequentially established based on analysis of multiple, sequentially received signal episodes.

In other examples, control circuit 206 may obtain signals and data for continuous transmission via telemetry circuit 208 at block 906 during a continuous telemetry session while operating in the VDI pacing mode. The motion signal and any other data may be transmitted continuously without waiting for a new signal episode request at block 908. Processor 52 may receive the transmitted data and extract signal episodes, e.g., 5 to 120 second episodes, from the transmitted data as needed for analyzing the signal episodes and establishing AVS pacing control parameters according to any of the examples described herein. During the continuous transmission of the motion signal and optionally the EGM signal, timing markers, sensed A4 event data, etc., pacemaker 14 may receive a command from external device 50 to adjust one or more AVS pacing control parameters based on the analysis performed so far by external device processor 52. Pacemaker 14 may implement the control parameter change by adjusting the one or more AVS pacing control parameters and continue operating in the VDI pacing mode and transmitting the motion signal and any other data until a verification command is received at block 910.

After receiving a verification command at block 910, control circuit 206 may receive at block 912 the AVS control parameters established by external device 50, which may include any of a PVABP, an A3 window ending time, early A4 sensing threshold amplitude, late A4 sensing threshold amplitude, pacing lower rate, automatic PVABP adjustment enabled, rate smoothing increment, and/or maximum tracking rate or any other AVS pacing control parameters described herein. Control circuit 206 may implement the received AVS pacing control parameters and, at block 914, control circuit 206 may switch to an atrial synchronous ventricular pacing mode, e.g., a VDD pacing mode, with the control parameters established by external device 50 in effect.

At block 914, control circuit 206 may deliver ventricular pacing in accordance with the implemented AVS pacing control parameters while operating in the atrial synchronous ventricular pacing mode. At block 915, control circuit 206 may log each ventricular event as being one of an AVS pacing pulse, a non-AVS pacing pulse (e.g., delivered at an LRI or an RSI in the absence of a sensed A4 event), or a sensed R-wave. Control circuit 206 may determine the percentage of AVS pacing pulses delivered out of all ventricular events logged over a predetermined time interval at block 916. The predetermined time interval may be 5 to 60 seconds, one to five minutes or any other specified time interval. In some examples, the predetermined time interval may be several hours, e.g., up to 24 hours. However, the predetermined time interval can be kept relatively short to enable verification of the established AVS pacing control parameters in cooperation with external device 50 during one, relatively short setup procedure that may be completed in 10 minutes or less or even 5 minutes or less.

Control circuit 206 may compare the AVS pacing pulse percentage to a threshold percentage at block 916. When the AVS pacing pulse percentage is less than the threshold percentage, e.g., less than 50%, 60%, 70%, 80% or other selected percentage, control circuit 206 may transmit new signal episode data to external device 50. External device 50 may perform the verification process of testing for various conditions and adjusting AVS pacing control parameters using the new signal episode data, e.g., as described above in conjunction with FIG. 13A and FIG. 13B. It is to be understood however, that in some examples, instead of transmitting signal episode data at block 918, pacemaker control circuit 206 may perform the verification process described in conjunction with FIGS. 13A and 13B. At block 920, control circuit 206 may receive a programming command from external device 50 to adjust one or more control parameters. Control circuit 206 may implement the adjusted control parameter(s) and continue delivering ventricular pacing according to the atrial synchronous ventricular pacing mode and the adjusted AVS pacing control parameter(s).

Control circuit 206 may return to block 916 to redetermine the AVS pacing percentage. When the AVS pacing percentage meets the threshold percentage at block 916, control circuit 206 may confirm the AVS pacing control parameter and that the set up process for establishing the AVS pacing control parameters is complete at block 922. Control circuit 206 may continue operating in the atrial synchronous ventricular pacing mode with the confirmed, established AVS pacing control parameters. Control circuit 206 may make adjustments to the control parameters during ongoing sensing and pacing operations, e.g., according to the techniques generally disclosed in the above-incorporated U.S. patent application Ser. No. 17/159,596 (Sheldon, et al.) and U.S. patent application Ser. No. 17/159,635 (Sheldon, et al.).

FIG. 15 is a diagram 1000 of cardiac signals in a signal episode that may be analyzed by processing circuitry of a medical device system for establishing AVS pacing control parameters according to some examples. A motion signal 1002 sensed by motion sensor 212, an EGM signal 1004 sensed by sensing circuit 204, and an ECG signal 1106 received by external device 50 are shown in diagram 1000. The diagram 1000 may represent a portion of a GUI displayed to user by display unit 54 to provide a visual representation of the cardiac signals and relative timing of ventricular pacing pulses 1010, the PVABP ending time 1012, the A3 window ending time 1014 and the timing of identified P-waves 1140, 1142, 1144, 1146 and 1148 (e.g., based on output from cardiac signal analyzer 51) during each cardiac cycle.

Three cardiac cycles 1130, 1132 and 1134 are shown, each beginning with a ventricular pacing pulse. An EGM R-wave 1008 and ECG R-wave 1018 representing the pacing-evoked ventricular depolarization follows each ventricular pacing pulse 1010. A1 signals corresponding to ventricular contraction during the systolic phase of each cardiac cycle 1130, 1132 and 1134 are observed following each R-wave. The ventricular pacing pulses 1010 are delivered in an asynchronous ventricular pacing mode such that the identified P-waves 1140, 1142, 1144, 1146 and 1148 occur at varying times during the three cardiac cycles 1130, 1132 and 1134 as shown.

Processor 52 (or pacemaker control circuit 206) may determine features of the motion signal 1002 during each cardiac cycle 1130, 1132, and 1134 for buffering in external device memory 53 (or pacemaker memory 210). In the example shown, the displayed cardiac cycles may be annotated with an A3 Max (the maximum amplitude of the motion signal 1002 during the A3 window 1150 extending from the PVABP ending time 1012 and the A3 window ending time 1014), A3 Time (the time of A3 Max from the starting ventricular pacing pulse 1010 of the respective cardiac cycle), A4 Max (the maximum amplitude of the motion signal 1002 during the A4 window 1152 extending from the A3 window ending time 1014 until the next ventricular pacing pulse) and the A4 Time (the time of A4 Max from the starting ventricular pacing pulse of the respective cardiac cycle). In other examples, other motion signal features may additionally or alternatively be determined from the motion signal 1002 during each cardiac cycle, such as the maximum peak amplitude during the PVABP, the time of a latest-negative going threshold crossing during the PVABP, the time of a latest-negative going threshold crossing during the A3 window or other amplitude-and/or timing-related features of the motion signal 1002. In still other examples, the motion signal features such as A3 Max, A4 Max, A3 Time and A4 Time may be determined only from cardiac cycles that are identified as being accepted cardiac cycles, e.g., LD P-wave cycles. Display unit 54 may annotate the displayed cardiac signals by displaying the determined motion signal features (or some of the motion signal features) in a GUI in some examples, e.g., as generally shown in FIG. 15.

Processor 52 (or pacemaker control circuit 206) may identify each cardiac cycle having a P-wave timing identified during a LD time period 1020. In the example shown, the first cardiac cycle 1130 may be rejected in some examples because P-wave 1140 is in the A3 window (after the PVABP ending time 1012 and before the A3 window ending time 1014), and no P-wave is identified in the LD time period 1020. Cardiac cycle 1130 could be identified as an ED P-wave cycle in some examples so that A3 Max may be used in some examples in setting the early A4 sensing threshold amplitude as described below in conjunction with FIG. 16. When a P-wave 1140 occurs during the A3 window, the A3 and A4 events may be fused (as indicated in FIG. 15) resulting in a high amplitude signal in motion signal 1102. The A3 Max determined to be 35 ADC units in cardiac cycle 1130 may correspond to the amplitude of fused A3 and A4 events such that the A3 Max of an ED P-wave cycle may be used in establishing an early A4 sensing threshold amplitude so that the fused A3+A4 event signal may be sensed by control circuit 206 of pacemaker 14.

Cardiac cycle 1132 may be accepted as a LD P-wave cycle because the P-wave 1144 occurs during the LD time period 1120. As observed in motion signal 1002, the A4 signal is a relatively high amplitude signal corresponding to the true atrial systolic event. The A3 signal is a relatively lower amplitude signal during the A3 window 1150. The features of motion signal 1002 sensed during cardiac cycle 1132 can be a reliable for use in establishing AVS pacing control parameters because the timing of P-wave 1144 during LD time period 1120 causes a clean A4 signal during the A4 window 1152. A4 Max (determined to be 33 ADS units in this example) during the A4 window 1152 of cardiac cycle 1132 may be determined and used by processor 52 in establishing the late A4 sensing threshold amplitude applied to the motion signal during A4 windows by pacemaker control circuit 206.

The A3 event during cardiac cycle 1132 may represent a true A3 event signal that is not altered or corrupted by an A4 event when the P-wave 1144 is during the LD time period 1120 and no other P-wave occurs in the A3 window 1150 (or in the late portion of the PVABP). Passive ventricular filling during early ventricular diastole associated with the A3 event signal is not altered by atrial mechanical systole. Accordingly, A3 Max (determined to be 9 ADC units in this example) during the A3 window 1150 of cardiac cycle 1132 may be determined and used in establishing the early A4 sensing threshold amplitude in some examples.

In cardiac cycle 1132, a second P-wave 1142 occurs during the PVABP. In some examples, a LD P-wave cycle is accepted regardless of whether a second P-wave occurs during the same cardiac cycle or not. In other examples, if a second P-wave occurs during a LD P-wave cycle, the cardiac cycle may be rejected or accepted depending on the timing of the second P-wave. In the case of cardiac cycle 1132, the second P-wave 1142 occurs relatively early in the PVABP, e.g., before or at the start of the T-wave 1019. When the P-wave occurs during the ventricular systolic phase, the atria may be contracting against a closed atrioventricular valve. This timing of atrial mechanical systole during ventricular systole may not alter the true A3 and true A4 signals that occur during ventricular diastole in a LD P-wave cycle, such as cardiac cycle 1132. As such, a LD P-wave cycle having a second P-wave 1142 that occurs relatively early during ventricular systole, e.g., during the first half of the PVABP or within the first 200, 300, 350, or 400 ms after the ventricular pacing pulse, may be accepted by processor 52 for use in establishing AVS pacing control parameters.

When a second P-wave occurs during a later portion of ventricular systole (or during the A3 window), however, the cardiac cycle may be rejected by processor 52 (or pacemaker control circuit 206) for use in establishing AVS pacing control parameters. In cardiac cycle 1134, a P-wave 1148 is identified in the LD time period 1120, but a second P-wave 1146 is identified during the PVABP coincident with the T-wave. In this case, atrial contraction during the late systolic/early diastolic phase of the ventricular cycle may result in some alteration or contamination of the motion signal during the A3 window because the A4 event associated with atrial contraction may be occurring together or in juxtaposition with the A2 and/or A3 events. The “atrial kick” that normally occurs with atrial contraction during late diastole and produces the A4 signal may not occur during early diastole due to the atria contracting against closed AV valves. The A3 Max may not be reliable due to influences of the mechanical atrial systolic event near the start of ventricular diastole. The cardiac cycle 1134 may be rejected in some examples due to the second P-wave 1146 occurring later than the early systolic period (e.g., the first half of the PVABP) even though P-wave 1148 occurs during the LD time period 1120. A3 Max is determined to be 17 ADC units for cardiac cycle 1134, which is in between A3 Max of 35 ADC units during the first cardiac cycle 1130 when A3 and A4 events are likely fused and the A3 Max of 9 ADC units during the second cardiac cycle 1132 when the A3 event is likely to be a true A3 event signal without alteration or contamination by an A4 event.

However, when multiple LD P-wave cycles are identified and accepted regardless of occurrences of second P-waves during the cardiac cycle, the influence of one altered A3 event signal due to possible contamination by an early A4 event may be minimized or reduced when a representative value of A3 Max (and other motion signal features) is determined from multiple LD P-wave cycles. As such, in some examples, cardiac cycle 1134 may be accepted as an LD P-wave cycle, and the motion signal sensed during cardiac cycle 1134 may be used in establishing AVS pacing control parameters.

The P-wave 1148 that occurs during the LD time period 1120 of cardiac cycle 1134 occurs relatively late, about 100 to 150 ms before the next ventricular pacing pulse ending A4 window 1152. As a result, the A4 signal occurring just prior to the end of A4 window 1152 may be truncated or altered due to the onset of ventricular systole. As such, in some examples, the LD time period 1120 may be defined to avoid accepting cardiac cycles in which the A4 event occurs very late, just prior to the next ventricular pacing pulse. A4 Max is determined to be 23 ADC units in cardiac cycle 1134, which is less than the A4 Max of 33 ADC units in the preceding LD P-wave cycle 1132, wherein the P-wave 1144 occurs very early in the LD time period, e.g., within the first 50 to 100 ms of the A4 window 1152. A P-wave identified after the LD time period 1120 and before the next ventricular pacing pulse can result in the A4 event being altered or corrupted by ventricular mechanical systole. In the example shown, LD time period 1120 extends from 975 ms to 1125 ms after the staring ventricular pacing pulse of the respective cardiac cycle. Depending on the asynchronous ventricular pacing rate, the starting and ending times of the LD time period 1120 may be adjusted to identify cardiac cycles in which the A3 event and the A4 event of the motion signal 1002 are expected to be unaltered or uncorrupted by other cardiac events, e.g., as represented by cardiac cycle 1132 in FIG. 15.

It is recognized, however, that in order to accumulate a threshold number of LD P-wave cycles, the LD time period 1120 may be defined to have as long of duration as possible to reasonable identify cardiac cycles in which true A3 events and true A4 events occur without alteration or corruption by other cardiac events. When a motion signal feature used in establishing an AVS pacing control parameter is determined from multiple LD P-wave cycles, averaging and or filtering of the motion signal feature values may be performed by the processing circuitry to reduce the influence of outliers and/or values falling in the upper and/or lower portion of the range of values of the motion signal feature. For example, the n lowest and/or n highest values may be discarded, the lower quartile and/or upper quartile of values may be discarded, values above and/or below a specified number of standard deviations from the mean may be discarded, or other methods may be used for filtering the motion signal feature values to obtain a representative motion signal feature value that is reliable for establishing AVS pacing control parameters.

FIG. 16 is a flow chart 1100 of a method that may be performed by processing circuitry of medical device system 10 (e.g., processor 52 and/or pacemaker control circuit 206) for accumulating motion signal feature data for establishing AVS pacing control parameters according to another example. As generally described above in conjunction with FIG. 6, pacemaker 14 may operate in a non-atrial tracking (asynchronous) ventricular pacing mode for obtaining signal episode at block 1104 including multiple cardiac cycles of the motion signal and at least one cardiac electrical signal (ECG and/or EGM) signal. At block 1106, cardiac signal analyzer 51 determines the timing of P-waves identified in the cardiac electrical signal. If a P-wave is in the LD time period, as determined by processor 52 at block 1108, processor 52 may determine if another P-wave occurs during the same cardiac cycle at block 1110. If another P-wave is not identified in the cardiac cycle by cardiac signal analyzer 51, processor 52 may accept the LD P-wave cycle at block 1114. Motion signal data may be determined and stored from the cardiac cycle at block 1116 for use in establishing AVS pacing control parameters at block 1130.

When cardiac signal analyzer 51 identifies a second P-wave in the same cardiac cycle, processor 52 may determine if the second P-wave timing is in an early systolic time period at block 1112. The early systolic time period may be the PVABP or an early portion thereof, e.g., the first 200 to 400 ms of the PVABP. In other examples, cardiac signal analyzer 51 may be configured to identify R-waves and/or T-waves in addition to P-waves from the input cardiac electrical signal. Cardiac signal analyzer 51 may output the timing of an identified R-wave and/or an identified T-wave during the cardiac cycle. When processor 52 determines that the P-wave timing is within a threshold time interval of the T-wave, processor 52 may determine that the P-wave timing is not in the early systolic time period at block 1112. Processor 52 may determine that a second P-wave in the same cardiac cycle is in early systole based on the P-wave timing from the delivered ventricular pacing pulse, an identified R-wave, and/or an identified T-wave in various examples.

When the second P-wave timing is in the early systolic time period, the cardiac cycle can be accepted by processor 52 as an LD P-wave cycle at block 1114. As described above in conjunction with FIG. 15, the motion signal sensed during the A3 window and A4 window is unlikely to be altered or corrupted by the early systolic P-wave, allowing processor 42 to determine reliable A3 Max and A4 Max values for use in establishing AVS pacing control parameters. When the second P-wave timing is not in the early systolic time period (“no” branch of block 1112), the cardiac cycle may be rejected at block 1124 by processor 52. A second P-wave occurring later in the cardiac cycle, e.g., during late systole or early diastole, may alter the true A3 event signal as described above in conjunction with FIG. 15.

Returning to block 1108, when a P-wave is not identified by cardiac signal analyzer during the LD time period, processor 52 may determine if a P-wave is identified in the ED time period at block 1120. As described above in conjunction with FIG. 15, a cardiac cycle, such as cardiac cycle 1130, having a P-wave identified during the A3 window (or another defined ED time period), may include a fused A3+A4 signal that can be used for establishing the early A4 sensing threshold amplitude. In this case, the ED P-wave cycle may be accepted at block 1122 and one or more motion signal features may be determined and stored in memory 53 for use establishing one or more AVS pacing control parameters. Processor 52 may use the A3 Max of ED P-wave cycles for establishing the early A4 sensing threshold, for example, but may exclude determining or using the A4 Max value for establishing the late A4 sensing threshold because a true A4 event does not occur during the A4 window of the accepted LD P-wave cycle.

After accepting or rejecting the cardiac cycle, processor 52 may determine if another cardiac cycle is available in the signal episode at block 1126 and/or if a threshold number of accepted cardiac cycles (e.g., a threshold number of accepted LD P-wave cycles) is reached at block 1128. When all cardiac cycles in the signal episode have been evaluated or a threshold number of accepted LD P-wave cycles has been reached, processor 52 may establish the AVS pacing control parameters at block 1130 based on the motion signal features determined from accepted cardiac cycles.

When both ED P-wave cycles and LD P-wave cycles are accepted, processor 52 may use the LD P-wave cycles for establishing the late A4 sensing threshold amplitude. For example, processor 52 may determine the late A4 sensing threshold amplitude that is less than all (or at least a specified percentage, e.g., 80%, 90% or other percentage) of A4 Max values determined from LD P-wave cycles. Processor 52 may use only the ED P-wave cycles or a combination of the ED P-wave cycles and the LD P-wave cycles for establishing the early A4 sensing threshold amplitude. For example, processor 52 may determine an early A4 sensing threshold amplitude to be less than all (or at least a specified percentage, e.g., 80%, 90% or other percentage) of A3 Max values determined from ED P-wave cycles. In other examples, processor 52 may determine the early A4 sensing threshold amplitude to be greater than all A3 Max values determined during LD P-wave cycles and less than at least a specified percentage of the A3 Max values determined from ED P-wave cycles.

The A3 window ending time may be established by processor 52 based on the A3 times and/or the A4 times determined from the LD P-wave cycles. For example, the A3 window ending time may be determined to be later than all (or a specified percentage of A3 times determined from the LD P-wave cycles. In other examples, processor 52 may determine the A3 window ending time to be earlier than all (or a specified percentage of) A4 times determined from the LD P-wave cycles. In still other examples, processor 52 may determine the A3 window ending time to be at a midpoint or other portion of the time interval between a representative A3 time and a representative A4 time (where the representative A3 time and representative A4 time may be a mean, median, maximum or minimum value of the respective A3 time or A4 time, as examples).

In other examples, processor 52 may establish the A3 window ending time based on the ED P-wave cycles or a combination of ED P-wave cycles and LD P-wave cycles. For example, processor 52 may determine the A3 window ending time to be greater than the A3 time in all (or a specified percentage of) ED P-wave cycles or greater than the A3 time in all (or a specified percentage of) ED P-wave cycles and LD P-wave cycles.

Processor 52 may establish a PVABP at block 1130 based on the A3 time determined during the ED P-wave cycles and/or LD P-wave cycles. For example, processor 52 may determine the PVABP to have a duration that ends prior to all (or a specified percentage of) the A3 times determined from the ED P-wave cycles, determined from the LD P-wave cycles, or a combination of both.

The foregoing illustrative examples for determining an AVS pacing control parameter based on accepted ED P-wave cycles or LD P-wave cycles or a combination of both are not intended to be limiting. It is to be understood that after identifying accepted LD P-wave cycles and ED P-wave cycles, processor 52 (or pacemaker control circuit 206) may establish AVS pacing control parameters according to a variety of methods based on the motion signal features determined from the accepted cardiac cycles in a manner that promotes reliable A4 event sensing by atrial event detector circuit 240 during atrial synchronous ventricular pacing.

Furthermore, it is contemplated that in some examples AVS pacing control parameters may be established by processor 52 based on as few as one accepted LD P-wave cycle. As shown in FIG. 15 and described above, when one LD P-wave cycle is identified such as cardiac cycle 1132, the A3 event signal during the A3 window and the A4 event signal during the A4 window provide reliable amplitude and timing information for establishing AVS pacing control parameters. In some examples, a single LD P-wave cycle having the P-wave timing during the LD time period and at least a threshold time interval earlier than the end of the A4 window (e.g., 100 ms to 200 ms before the next ventricular pacing pulse) may be an optimal LD P-wave cycle for use in establishing one or more AVS pacing control parameters.

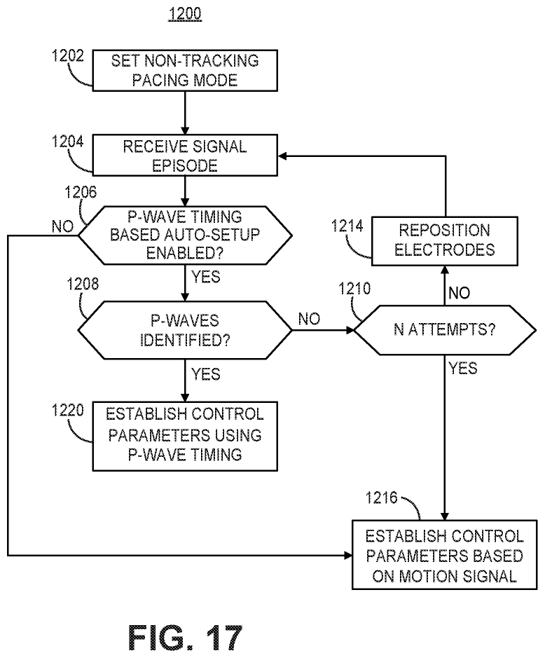

FIG. 17 is a flow chart 1200 of a method for establishing AVS pacing control parameters according to some examples. Pacemaker 14 operates in a non-atrial tracking (asynchronous) ventricular pacing mode at block 1202. At block 1204 processing circuitry (e.g., pacemaker control circuit 206 or external device processor 52) receives a signal episode including the motion signal (for one or more motion signal sensing vectors) and at least one cardiac electrical signal (ECG and/or EGM signal(s)). At block 1206, the processing circuitry may determine if P-wave timing based auto-setup for establishing AVS pacing control parameters is enabled. A user may program the auto-setup method to be used for establishing AVS pacing control parameters. For example, a user may enable P-wave timing based auto-setup when ECG electrodes are positioned for providing an ECG signal input to cardiac signal analyzer 51. A user may enable P-wave timing based auto-setup as the preferred auto-setup method, or P-wave timing based auto-setup may be enabled as a default auto-setup method. If P-wave timing based auto-setup is not enabled, the processing circuitry may advance to block 1216 to establish AVS control parameters based on the motion signal without requiring identification of P-wave timing markers.

When P-wave timing based auto-setup is enabled at block 1206, the processing circuitry may advance to block 1208 to determine if P-waves are being identified from the cardiac electrical signal(s) received in the signal episode. P-wave identification may depend on a number of factors such as electrode positioning and cardiac electrical signal quality. In some cases, P-waves may not be reliably identified by cardiac signal analyzer 51. For example, cardiac signal analyzer 51 may output a P-wave timing marker with a level of confidence. If the level of confidence of an output P-wave timing marker is less than a threshold level, e.g., less than 90%, 80%, 70% or other specified threshold level, the processing circuitry may determine that no P-wave is identified. The processing circuitry may determine that P-waves are not being identified reliably at block 1208 when less than a threshold number or threshold percentage of P-wave timing markers in the signal episode are associated with at least threshold level of confidence. For example, when less than 10%, 20%, 30%, 40%, 50% or other threshold number of cardiac cycles include an identified P-wave with a level of confidence that is at least 80% (or other selected level), the processing circuitry may determine that P-waves are not being reliably identified at block 1208.

In some examples, the processing circuitry may attempt the P-wave timing based auto-setup procedure for a maximum number (N) of attempts. If N attempts have not been made, the processing circuitry may return to block 1204 to obtain another signal episode. In some examples, the processing circuitry may cause the external device display unit 54 to display a message or prompt to the clinician or other user to reposition the ECG electrodes at block 1214. In some cases, repositioning of surface electrodes may improve the P-wave signal quality to enable reliable P-wave identification from a subsequent signal episode.

When the processing circuitry determines that P-waves are being identified reliably at block 1208 (e.g., based on the example criteria described above), the processing circuitry may advance to block 1220 to establish the AVS pacing control parameters using the P-wave timing according to the techniques disclosed herein. For instance, based on P-wave timing, the processing circuitry may identify one or more accepted cardiac cycles from which motion signal features are determined for establishing at least one AVS pacing control parameters. In some examples, multiple P-wave timing markers may be used for determining PPIs used in setting one or more AVS pacing control parameters, e.g., as described above in conjunction with FIG. 12.

When a maximum number of attempts at P-wave timing based auto-setup have been made (“yes” branch of block 1210), or when P-wave timing based auto-setup is disabled (“no” branch of block 1206), the processing circuitry may advance to block 1216 for establishing control parameters based on the motion signal without requiring identification of P-waves. Example techniques that can be used for establishing AVS pacing control parameters during an auto-setup procedure that does not require identification of P-waves are generally disclosed in in U.S. patent application Ser. No. 16/703,047 (Splett, et al.) and U.S. patent application Ser. No. 16/703,320 (Splett, et al.). For example, processing circuitry may determine motion signal features from multiple cardiac cycles for determining a frequency distribution of the motion signal features during asynchronous ventricular pacing. One or more AVS pacing control parameters may be determined from the frequency distribution(s) of the motion signal features.

It should be understood that, depending on the example, certain acts or events of any of the methods described herein can be performed in a different sequence, may be added, merged, or left out altogether (e.g., not all described acts or events are necessary for the practice of the method). Moreover, in certain examples, acts or events may be performed concurrently, e.g., through multi-threaded processing, interrupt processing, or multiple processors, rather than sequentially. In addition, while certain aspects of this disclosure are described as being performed by a single circuit or unit for purposes of clarity, it should be understood that the techniques of this disclosure may be performed by a combination of units or circuits associated with, for example, a medical device.

In one or more examples, the functions described may be implemented in hardware, software, firmware, or any combination thereof. If implemented in software, the functions may be stored as one or more instructions or code on a computer-readable medium and executed by a hardware-based processing unit. Computer-readable media may include computer-readable storage media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPLAs), or other equivalent integrated or discrete logic circuitry. Accordingly, the term “processor,” as used herein may refer to any of the foregoing structure or any other structure suitable for implementation of the techniques described herein. Also, the techniques could be fully implemented in one or more circuits or logic elements.

Thus, a medical device has been presented in the foregoing description with reference to specific examples. It is to be understood that various aspects disclosed herein may be combined in different combinations than the specific combinations presented in the accompanying drawings. It is appreciated that various modifications to the referenced examples may be made without departing from the scope of the disclosure and the following claims.

The following examples are a non-limiting list of clauses in accordance with one or more techniques of this disclosure.

Example 1. A medical device comprising: processing circuitry configured to: receive a cardiac motion signal sensed over a first signal episode; receive at least one cardiac electrical signal; determine that a first P-wave of the at least one cardiac electrical signal occurs in a diastolic period of a first cardiac cycle of the first signal episode; in response to the first P-wave being in the diastolic period of the first cardiac cycle, determine at least a first feature of the cardiac motion signal sensed during the first cardiac cycle; and establish a first control parameter based on at least the first feature, the first control parameter used for controlling delivery of atrial synchronous ventricular pacing.

Example 2. The medical device of Example 1, wherein the processing circuitry being further configured to: input the at least one cardiac electrical signal to a cardiac signal; output a P-wave timing marker by the cardiac signal analyzer in response an identified P-wave; and determine that the first P-wave is in the diastolic period of the first cardiac cycle based on the P-wave timing marker output by the cardiac signal analyzer.

Example 3. The medical device of any of Examples 1-2, wherein the processing circuitry is further configured to: receive the cardiac motion signal and the at least one cardiac electrical signal sensed over a plurality of cardiac cycles of the first signal episode; identify a plurality of P-waves including the first P-wave over the plurality of cardiac cycles; identify at least one diastolic P-wave cycle among the plurality of cardiac cycles, each identified diastolic P-wave cycle being a cardiac cycle that is associated with a P-wave of the identified plurality of P-waves being during a diastolic period of the respective cardiac cycle; determine a plurality of features of the motion signal comprising the first feature from the plurality of identified diastolic P-wave cycles; and establish the first control parameter based on the plurality of features.

Example 4. The medical device of any of Examples 1-3, wherein the processing circuitry is further configured to receive the motion signal over the plurality of cardiac cycles comprising asynchronous ventricular pacing pulses.

Example 5. The medical device of any of Examples 1-4, wherein the processing circuitry is further configured to: receive the cardiac motion signal and the at least one cardiac electrical signal sensed over a second signal episode during which atrial synchronous ventricular pacing is delivered according to the established first control parameter; identify a second P-wave in the at least one cardiac electrical signal; determine that the second P-wave occurs in a diastolic period of a second cardiac cycle; in response to the second P-wave being in the diastolic period of the second cardiac cycle, determine at least a second feature of the cardiac motion signal sensed during the second cardiac cycle; and establish a second control parameter based on at least the second feature, the second control parameter used for controlling the delivery of atrial synchronous ventricular pacing.

Example 6. The medical device of any of Examples 1-5, wherein the processing circuitry is further configured to establish the first control parameter by establishing an atrial event sensing control parameter used for sensing atrial events from the motion signal.

Example 7. The medical device of Example 6, wherein the processing circuit is further configured to: determine the first feature of the cardiac motion signal sensed during the first cardiac cycle by determining a maximum amplitude of the motion signal during the diastolic period of the first cardiac cycle; and establish the first control parameter based on at least the first feature by establishing a motion signal sensing vector for sensing the motion signal.

Example 8. The medical device of any of Examples 1-6, wherein the processing circuit is further configured to: determine the first feature of the cardiac motion signal sensed during the first cardiac cycle by determining a maximum amplitude of the motion signal during the diastolic period of the first cardiac cycle; and establish the first control parameter based on at least the first feature by establishing an atrial event sensing threshold amplitude for sensing atrial events from the motion signal.

Example 9. The medical device of Example 8, wherein the processing circuitry is further configured to: determine that the first P-wave occurs in a late diastolic period of the first cardiac cycle; and establish the first control parameter based on at least the first feature by establishing a late atrial event sensing threshold amplitude that is applied to the motion signal during an atrial event window for sensing atrial events from the motion signal.

Example 10. The medical device of any of Examples 8-9, wherein the processing circuitry is further configured to: identify a second P-wave in the at least one cardiac electrical signal; determine that the second P-wave occurs in an early diastolic period of a second cardiac cycle of the first signal episode; in response to the second P-wave being in the early diastolic period of the second cardiac cycle, determine at least a second feature of the cardiac motion signal sensed during the second cardiac cycle; and establish a second control parameter based on at least the second feature by establishing an early atrial event sensing threshold amplitude that is applied to the motion signal during a passive ventricular filling window for sensing atrial events from the motion signal.

Example 11. The medical device of any of Examples 8-9, wherein the processing circuitry is further configured to: determine that the first P-wave occurs in a late diastolic period of the first cardiac cycle; determine a second feature of the cardiac motion signal sensed during the first cardiac cycle by determining a maximum amplitude of the motion signal during a passive ventricular filling window of the first cardiac cycle; and establish a second control parameter based on at least the second feature by establishing an early atrial event sensing threshold amplitude that is applied to the motion signal during the passive ventricular filling window for sensing atrial events from the motion signal.

Example 12. The medical device of Example 6, wherein the processing circuitry is further configured to: set a test threshold amplitude; determine the first feature of the cardiac motion signal sensed during the first cardiac cycle by determining a latest threshold crossing of the test threshold amplitude during a passive ventricular filling window of the first cardiac cycle; and establish the first control parameter based on at least the first feature by establishing a passive ventricular filling window ending time used for sensing atrial events from the motion signal.

Example 13. The medical device of Example 6, wherein the processing circuitry is further configured to: determine the first feature of the cardiac motion signal sensed during the first cardiac cycle by determining a time of a maximum amplitude of the cardiac motion signal during the diastolic period of the first cardiac cycle; and establish the first control parameter based on at least the first feature by establishing a passive ventricular filling window ending time used for sensing atrial events from the motion signal.

Example 14. The medical device of Example 6, wherein the processing circuitry is further configured to: determine the first feature of the cardiac motion signal sensed during the first cardiac cycle by determining a maximum amplitude of the motion signal during the first cardiac cycle; and establish the first control parameter based on at least the first feature by establishing a post-ventricular atrial blanking period.

Example 15. The medical device of any of Examples 1-14, wherein the processing circuitry is further configured to: receive the cardiac motion signal and the at least one cardiac electrical signal sensed over a plurality of cardiac cycles of the first signal episode; identify a plurality of P-waves over the plurality of cardiac cycles; determine at least one PP interval between consecutively identified P-waves of the plurality of P-waves; and establish a second control parameter based on the at least one PP interval for controlling delivery of atrial synchronous ventricular pacing.

Example 16. The medical device of Example 15, wherein the processing circuitry is further configured to establish the second control parameter by establishing at least one of: a pacing lower rate, a rate smoothing increment, a post-ventricular atrial blanking period, and a maximum atrial tracking rate.

Example 17. The medical device of any of Examples 1-16, wherein the processing circuitry is further configured to: determine that a threshold percentage of atrioventricular synchronous pacing pulses is not met; and in response to determining that the threshold percentage of atrioventricular synchronous pacing pulses is not met, adjust at least one atrial synchronous pacing control parameter.

Example 18. The medical device of Example 17, wherein the processing circuitry is further configured to adjust the at least one atrial synchronous pacing control parameter by adjusting one of a plurality of control parameters that includes the first control parameter.

Example 19. The medical device of any of Examples 17-18, wherein the processing circuitry is further configured to adjust the at least one control parameter by adjusting at least one of: a post-ventricular atrial blanking period, a passive ventricular filling window ending time, an early atrial event sensing threshold amplitude, or a late atrial event sensing threshold amplitude.

Example 20. The medical device of any of Examples 17-19, wherein the processing circuitry is further configured to adjust the at least one control parameter by adjusting at least one of: a pacing lower rate, a rate smoothing increment, a maximum atrial tracking rate.

Example 21. The medical device of any of Examples 1-20, wherein the processing circuitry is further configured to determine that the first P-wave occurs in a diastolic period of the first cardiac cycle of the first signal episode by: applying a late diastolic threshold time; and determining that the first P-wave occurs after the late diastolic threshold time and before a ventricular electrical event that ends the first cardiac cycle.

Example 22. The medical device of any of Examples 1-21, wherein the processing circuitry is further configured to: receive the cardiac motion signal and the at least one cardiac electrical signal sensed over a plurality of cardiac cycles of the first signal episode; determine from the at least one cardiac electrical signal that less than a threshold number of the plurality of cardiac cycles are identified as diastolic P-wave cycles; and adjust at least one asynchronous ventricular pacing interval in response to less than the threshold number of the plurality of cardiac cycles being identified as diastolic P-wave cycles.

Example 23. The medical device of any of Examples 1-22, wherein the processing circuitry is further configured to: determine a low confidence of P-wave identification in the at least one cardiac electrical signal; and establish the first control parameter based on the cardiac motion signal sensed over a plurality of cardiac cycles.

Example 24. The medical device of any of Examples 1-23, further comprising a telemetry circuit configured to transmit a programming command comprising the established first control parameter.

Example 25. The medical device of any of Examples 1-23, further comprising a pulse generator configured to deliver atrial synchronous ventricular pacing according to the first control parameter.

Example 26. A non-transitory computer readable medium storing instructions which, when executed by processing circuitry of a medical device, cause the medical device to: receive a cardiac motion signal sensed over a first signal episode; receive at least one cardiac electrical signal; determine that a first P-wave of the at least one cardiac electrical signal occurs in a diastolic period of a first cardiac cycle of the first signal episode; in response to the first P-wave being in the diastolic period of the first cardiac cycle, determine at least a first feature of the cardiac motion signal sensed during the first cardiac cycle; and establish a first control parameter based on at least the first feature, the first control parameter used for controlling delivery of atrial synchronous ventricular pacing.

Example 27. The non-transitory computer readable medium of Example 26, wherein the instructions further cause the medical device to: input the at least one cardiac electrical signal to a cardiac signal analyzer; output a P-wave timing marker by the cardiac signal analyzer in response an identified P-wave; and determine that the first P-wave is in the diastolic period of the first cardiac cycle based on the P-wave timing marker output by the cardiac signal analyzer.

Example 28. The non-transitory computer readable medium of any of Examples 26-27, wherein the instructions further cause the medical device to: receive the cardiac motion signal and the at least one cardiac electrical signal sensed over a plurality of cardiac cycles of the first signal episode; identify a plurality of P-waves including the first P-wave over the plurality of cardiac cycles; identify at least one diastolic P-wave cycle among the plurality of cardiac cycles, each identified diastolic P-wave cycle being a cardiac cycle that is associated with a P-wave of the identified plurality of P-waves being during a diastolic period of the respective cardiac cycle; determine a plurality of features of the motion signal comprising the first feature from the plurality of identified diastolic P-wave cycles; and establish the first control parameter based on the plurality of features.

Example 29. The non-transitory computer readable medium of any of Examples 26-28, wherein the instructions further cause the medical device to receive the motion signal over the plurality of cardiac cycles comprising asynchronous ventricular pacing pulses.

Example 30. The non-transitory computer readable medium of any of Examples 26-29, wherein the instructions further cause the medical device to: receive the cardiac motion signal and the at least one cardiac electrical signal sensed over a second signal episode during which atrial synchronous ventricular pacing is delivered according to the established first control parameter; identify a second P-wave in the at least one cardiac electrical signal; determine that the second P-wave occurs in a diastolic period of a second cardiac cycle; in response to the second P-wave being in the diastolic period of the second cardiac cycle, determine at least a second feature of the cardiac motion signal sensed during the second cardiac cycle; and establish a second control parameter based on at least the second feature, the second control parameter used for controlling delivery of the atrial synchronous ventricular pacing.

Example 31. The non-transitory computer readable medium of any of Examples 26-30, wherein the instructions further cause the medical device to establish the first control parameter by establishing an atrial event sensing control parameter used for sensing atrial events from the motion signal.

Example 32. The non-transitory computer readable medium of Example 31, wherein the instructions further cause the medical device to: determine the first feature of the cardiac motion signal sensed during the first cardiac cycle by determining a maximum amplitude of the motion signal during the diastolic period of the first cardiac cycle; and establish the first control parameter based on at least the first feature by establishing a motion signal sensing vector for sensing the motion signal.

Example 33. The non-transitory computer readable medium of Example 31, wherein the instructions further cause the medical device to: determine the first feature of the cardiac motion signal sensed during the first cardiac cycle by determining a maximum amplitude of the motion signal during the diastolic period of the first cardiac cycle; and establish the first control parameter based on at least the first feature by establishing an atrial event sensing threshold amplitude for sensing atrial events from the motion signal.

Example 34. The non-transitory computer readable medium of Example 33, wherein the instructions further cause the medical device to: determine that the first P-wave occurs in a late diastolic period of the first cardiac cycle; and establish the first control parameter based on at least the first feature by establishing a late atrial event sensing threshold amplitude that is applied to the motion signal during an atrial event window for sensing atrial events from the motion signal.

Example 35. The non-transitory computer readable medium of any of Examples 33-34, wherein the instructions further cause the medical device to: identify a second P-wave in the at least one cardiac electrical signal; determine that the second P-wave occurs in an early diastolic period of a second cardiac cycle of the first signal episode; in response to the second P-wave being in the early diastolic period of the second cardiac cycle, determine at least a second feature of the cardiac motion signal sensed during the second cardiac cycle; and establish a second control parameter based on at least the second feature by establishing an early atrial event sensing threshold amplitude that is applied to the motion signal during a passive ventricular filling window for sensing atrial events from the motion signal.

Example 36. The non-transitory computer readable medium of any of Examples 33-34, wherein the instructions further cause the medical device to: determine that the first P-wave occurs in a late diastolic period of the first cardiac cycle; determine a second feature of the cardiac motion signal sensed during the first cardiac cycle by determining a maximum amplitude of the motion signal during a passive ventricular filling window of the first cardiac cycle; and establish a second control parameter based on at least the second feature by establishing an early atrial event sensing threshold amplitude that is applied to the motion signal during the passive ventricular filling window for sensing atrial events from the motion signal.

Example 37. The non-transitory computer readable medium of Example 31, wherein the instructions further cause the medical device to: set a test threshold amplitude; determine the first feature of the cardiac motion signal sensed during the first cardiac cycle by determining a latest threshold crossing of the test threshold amplitude during a passive ventricular filling window of the first cardiac cycle; and establish the first control parameter based on at least the first feature by establishing a passive ventricular filling window ending time used for sensing atrial events from the motion signal.

Example 38. The non-transitory computer readable medium of Example 31, wherein the instructions further cause the medical device to: determine the first feature of the cardiac motion signal sensed during the first cardiac cycle by determining a time of a maximum amplitude of the cardiac motion signal during the diastolic period of the first cardiac cycle; and establish the first control parameter based on at least the first feature by establishing a passive ventricular filling window ending time used for sensing atrial events from the motion signal.

Example 39. The non-transitory computer readable medium of Example 31, wherein the instructions further cause the medical device to: determine the first feature of the cardiac motion signal sensed during the first cardiac cycle by determining a maximum amplitude of the motion signal during the first cardiac cycle; and establish the first control parameter based on at least the first feature by establishing a post-ventricular atrial blanking period.

Example 40. The non-transitory computer readable medium of any of Examples 26-39, wherein the instructions further cause the medical device to: receive the cardiac motion signal and the at least one cardiac electrical signal sensed over a plurality of cardiac cycles of the first signal episode; identify a plurality of P-waves over the plurality of cardiac cycles; determine at least one PP interval between consecutively identified P-waves of the plurality of P-waves; and establish a second control parameter based on the at least one PP interval for controlling delivery of atrial synchronous ventricular pacing.

Example 41. The non-transitory computer readable medium of Example 40, wherein the instructions further cause the medical device to establish the second control parameter by establishing at least one of: a pacing lower rate, a rate smoothing increment, a post-ventricular atrial blanking period, and a maximum atrial tracking rate.

Example 42. The non-transitory computer readable medium of any of Example 26-41, wherein the instructions further cause the medical device to: determine that a threshold percentage of atrioventricular synchronous pacing pulses is not met; and in response to determining that the threshold percentage of atrioventricular synchronous pacing pulses is not met, adjust at least one atrial synchronous pacing control parameter.

Example 43. The non-transitory computer readable medium of Example 42, wherein the instructions further cause the medical device to adjust the at least one atrial synchronous pacing control parameter by adjusting one of a plurality of control parameters that includes the first control parameter.

Example 44. The non-transitory computer readable medium of any of Examples 42-43, wherein the instructions further cause the medical device to adjust the at least one control parameter by adjusting at least one of: a post-ventricular atrial blanking period, a passive ventricular filling window ending time, an early atrial event sensing threshold amplitude, or a late atrial event sensing threshold amplitude.

Example 45. The non-transitory computer readable medium of any of Examples 42-44, wherein the instructions further cause the medical device to adjust the at least one control parameter by adjusting at least one of: a pacing lower rate, a rate smoothing increment, a maximum atrial tracking rate.

Example 46. The non-transitory computer readable medium of any of Examples 26-45, wherein the instructions further cause the medical device to determine that the first P-wave occurs in a diastolic period of the first cardiac cycle of the first signal episode by: applying a late diastolic threshold time; and determining that the first P-wave occurs after the late diastolic threshold time and before a ventricular electrical event that ends the first cardiac cycle.

Example 47. The non-transitory computer readable medium of any of Example 26-46, wherein the instructions further cause the medical device to: receive the cardiac motion signal and the at least one cardiac electrical signal sensed over a plurality of cardiac cycles of the first signal episode; determine from the at least one cardiac electrical signal that less than a threshold number of the plurality of cardiac cycles are identified as diastolic P-wave cycles; and adjust at least one asynchronous ventricular pacing interval in response to less than the threshold number of the plurality of cardiac cycles being identified as diastolic P-wave cycles.

Example 48. The non-transitory computer readable medium of any of Example 26-47, wherein the instructions further cause the medical device to: determine a low confidence of P-wave identification in the at least one cardiac electrical signal; and establish the first control parameter based on the cardiac motion signal sensed over a plurality of cardiac cycles.

Example 49. The non-transitory computer readable medium of any of Examples 26-48, wherein the instructions further cause the medical device to transmit a programming command comprising the established first control parameter.

Example 50. The non-transitory computer readable medium of any of Examples 26-48, wherein the instructions further cause the medical device to deliver atrial synchronous ventricular pacing according to the first control parameter.

Example 51. A method comprising: receiving a cardiac motion signal sensed over a signal episode; receiving at least one cardiac electrical signal; determining that a P-wave of the at least cardiac electrical signal occurs in a diastolic period of a cardiac cycle of the signal episode; in response to the P-wave being in the diastolic period of the cardiac cycle, determining at least one feature of the cardiac motion signal sensed during the cardiac cycle; and establishing an atrial synchronous ventricular pacing control parameter based on the at least one feature.

Example 52. A medical device, comprising: a motion sensor configured to: sense a cardiac motion signal; a pulse generator configured to generate ventricular pacing pulses; and a telemetry circuit configured to: transmit a signal episode of the motion signal; and receive an established control parameter from another medical device; and a control circuit configured to operate in an atrial synchronous ventricular pacing mode according to the established control parameter by: sensing atrial events from the cardiac motion signal; controlling the pulse generator to deliver atrial synchronous ventricular pacing pulses in response to sensing the atrial events; determine that a percentage of atrial synchronous ventricular pacing pulses out of a plurality of ventricular events is greater than a threshold percentage; and confirm the established control parameter for use in controlling atrial synchronous pacing.

1. A medical device system comprising:
processing circuitry configured to:
receive a cardiac motion signal sensed over a first signal episode;
receive at least one cardiac electrical signal;
determine that a first P-wave of the at least one cardiac electrical signal occurs in a diastolic period of a first cardiac cycle of the first signal episode;
in response to the first P-wave being in the diastolic period of the first cardiac cycle, determine at least a first feature of the cardiac motion signal sensed during the first cardiac cycle; and
establish at least a first control parameter based on at least the first feature, at least the first control parameter used for controlling delivery of atrial synchronous ventricular pacing.

2. The medical device system of claim 1, wherein the processing circuitry being further configured to:
input the at least one cardiac electrical signal to a cardiac signal analyzer;
output a P-wave timing marker by the cardiac signal analyzer in response an identified P-wave; and
determine that the first P-wave is in the diastolic period of the first cardiac cycle based on the P-wave timing marker output by the cardiac signal analyzer.

3. The medical device system of claim 1 wherein the processing circuitry is further configured to:
receive the cardiac motion signal and the at least one cardiac electrical signal sensed over a plurality of cardiac cycles of the first signal episode;
identify a plurality of P-waves including the first P-wave over the plurality of cardiac cycles;
identify at least one diastolic P-wave cycle among the plurality of cardiac cycles, each identified diastolic P-wave cycle being a cardiac cycle that is associated with a P-wave of the identified plurality of P-waves being during a diastolic period of the respective cardiac cycle;
determine a plurality of features of the motion signal comprising the first feature from the plurality of identified diastolic P-wave cycles; and
establish at least the first control parameter based on the plurality of features.

4. The medical device system of claim 1 wherein the processing circuitry is further configured to receive the motion signal over the plurality of cardiac cycles comprising asynchronous ventricular pacing pulses.

5. The medical device system of claim 1 wherein the processing circuitry is further configured to:
receive the cardiac motion signal and the at least one cardiac electrical signal sensed over a second signal episode during which atrial synchronous ventricular pacing is delivered according to at least the first control parameter established based on at least the first feature;
identify a second P-wave in the at least one cardiac electrical signal;
determine that the second P-wave occurs in a diastolic period of a second cardiac cycle;
in response to the second P-wave being in the diastolic period of the second cardiac cycle, determine at least a second feature of the cardiac motion signal sensed during the second cardiac cycle; and
establish a second control parameter based on at least the second feature, the second control parameter used for controlling the delivery of atrial synchronous ventricular pacing.

6. The medical device system of claim 1 wherein the processing circuitry is further configured to establish at least the first control parameter by establishing an atrial event sensing control parameter used for sensing atrial events from the motion signal.

7. The medical device system of claim 6, wherein the processing circuit is further configured to:
determine the first feature of the cardiac motion signal sensed during the first cardiac cycle by determining a maximum amplitude of the motion signal during the diastolic period of the first cardiac cycle; and
establish at least the first control parameter based on at least the first feature by establishing a motion signal sensing vector for sensing the motion signal.

8. The medical device system of claim 6, wherein the processing circuit is further configured to:
determine the first feature of the cardiac motion signal sensed during the first cardiac cycle by determining a maximum amplitude of the motion signal during the diastolic period of the first cardiac cycle; and
establish at least the first control parameter based on at least the first feature by establishing an atrial event sensing threshold amplitude for sensing atrial events from the motion signal.

9. The medical device system of claim 8, wherein the processing circuitry is further configured to:
determine that the first P-wave occurs in a late diastolic period of the first cardiac cycle; and
establish at least the first control parameter based on at least the first feature by establishing a late atrial event sensing threshold amplitude that is applied to the motion signal during an atrial event window for sensing atrial events from the motion signal.

10. The medical device system of claim 8 wherein the processing circuitry is further configured to:
identify a second P-wave in the at least one cardiac electrical signal;
determine that the second P-wave occurs in an early diastolic period of a second cardiac cycle of the first signal episode;
in response to the second P-wave being in the early diastolic period of the second cardiac cycle, determine at least a second feature of the cardiac motion signal sensed during the second cardiac cycle; and
establish a second control parameter based on at least the second feature by establishing an early atrial event sensing threshold amplitude that is applied to the motion signal during a passive ventricular filling window for sensing atrial events from the motion signal.

11. The medical device system of claim 8 wherein the processing circuitry is further configured to:
determine that the first P-wave occurs in a late diastolic period of the first cardiac cycle;
determine a second feature of the cardiac motion signal sensed during the first cardiac cycle by determining a maximum amplitude of the motion signal during a passive ventricular filling window of the first cardiac cycle; and
establish a second control parameter based on at least the second feature by establishing an early atrial event sensing threshold amplitude that is applied to the motion signal during the passive ventricular filling window for sensing atrial events from the motion signal.

12. The medical device system of claim 6, wherein the processing circuitry is further configured to:
set a test threshold amplitude;
determine the first feature of the cardiac motion signal sensed during the first cardiac cycle by determining a latest threshold crossing of the test threshold amplitude during a passive ventricular filling window of the first cardiac cycle; and
establish at least the first control parameter based on at least the first feature by establishing a passive ventricular filling window ending time used for sensing atrial events from the motion signal.

13. The medical device system of claim 6, wherein the processing circuitry is further configured to:
determine the first feature of the cardiac motion signal sensed during the first cardiac cycle by determining a time of a maximum amplitude of the cardiac motion signal during the diastolic period of the first cardiac cycle; and
establish at least the first control parameter based on at least the first feature by establishing a passive ventricular filling window ending time used for sensing atrial events from the motion signal.

14. The medical device system of claim 6, wherein the processing circuitry is further configured to:
determine the first feature of the cardiac motion signal sensed during the first cardiac cycle by determining a maximum amplitude of the motion signal during the first cardiac cycle; and
establish at least the first control parameter based on at least the first feature by establishing a post-ventricular atrial blanking period.

15. The medical device system of claim 1 wherein the processing circuitry is further configured to:
receive the cardiac motion signal and the at least one cardiac electrical signal sensed over a plurality of cardiac cycles of the first signal episode;
identify a plurality of P-waves over the plurality of cardiac cycles;
determine at least one PP interval between consecutively identified P-waves of the plurality of P-waves; and
establish a second control parameter based on the at least one PP interval for controlling delivery of atrial synchronous ventricular pacing.

16. The medical device system of claim 1 further comprising:
a pulse generator configured to deliver the atrial synchronous ventricular pacing; and
a control circuit configured to operate according to at least the first control parameter for controlling the pulse generator to deliver the atrial synchronous ventricular pacing.

17. Non-transitory computer readable storage media storing instructions that, when executed by a medical device system, cause the medical device system to:
receive a cardiac motion signal sensed over a first signal episode;
receive at least one cardiac electrical signal;
determine that a first P-wave of the at least one cardiac electrical signal occurs in a diastolic period of a first cardiac cycle of the first signal episode;
in response to the first P-wave being in the diastolic period of the first cardiac cycle, determine at least a first feature of the cardiac motion signal sensed during the first cardiac cycle; and
establish at least a first control parameter based on at least the first feature, at least the first control parameter used for controlling delivery of atrial synchronous ventricular pacing.

18. The non-transitory computer readable storage media of claim 17 wherein the instructions further cause the medical device system to:
receive the cardiac motion signal and the at least one cardiac electrical signal sensed over a plurality of cardiac cycles of the first signal episode;
identify a plurality of P-waves including the first P-wave over the plurality of cardiac cycles;
identify at least one diastolic P-wave cycle among the plurality of cardiac cycles, each identified diastolic P-wave cycle being a cardiac cycle that is associated with a P-wave of the identified plurality of P-waves being during a diastolic period of the respective cardiac cycle;
determine a plurality of features of the motion signal comprising the first feature from the plurality of identified diastolic P-wave cycles; and
establish at least the first control parameter based on the plurality of features.

19. The non-transitory computer readable storage media of claim 17 wherein the instructions further cause the medical device system to receive the motion signal over the plurality of cardiac cycles comprising asynchronous ventricular pacing pulses.

20. The non-transitory computer readable storage media of claim 17 wherein the instructions further cause the medical device system to establish at least the first control parameter by establishing an atrial event sensing control parameter used for sensing atrial events from the motion signal.
